SEQUENCE LISTING (1274 sequences)

SEQ ID NO: 1 - DNA, 19 bp, synthetic construct
  misc_feature 1..19: note = single stranded DNA
  source 1..19: mol_type = other DNA
tcacctcatcccgcgaagc

SEQ ID NO: 2 - RNA, 21 nt, synthetic construct
  misc_feature 1..21: note = artificial sequence
  source 1..21: mol_type = other RNA
tgccctttgacctcatggtgt

SEQ ID NO: 3 - RNA, 21 nt, synthetic construct
  misc_feature 1..21: note = artificial sequence
  source 1..21: mol_type = other RNA
cctcttcatccagacagacaa

SEQ ID NO: 4 - RNA, 21 nt, synthetic construct
  misc_feature 1..21: note = artificial sequence
  source 1..21: mol_type = other RNA
ccctttgacctcatggtgttc

SEQ ID NO: 5 - RNA, 21 nt, synthetic construct
  misc_feature 1..21: note = artificial sequence
  source 1..21: mol_type = other RNA
acctcttcatccagacagaca

SEQ ID NO: 6 - RNA, 21 nt, synthetic construct
  misc_feature 1..21: note = artificial sequence
  source 1..21: mol_type = other RNA
gcgggtacctcttcatccaga

SEQ ID NO: 7 - RNA, 21 nt, synthetic construct
  misc_feature 1..21: note = artificial sequence
  source 1..21: mol_type = other RNA
gacagacaagaccatctacac

SEQ ID NO: 8 - RNA, 21 nt, synthetic construct
  misc_feature 1..21: note = artificial sequence
  source 1..21: mol_type = other RNA
tccagacagacaagaccatct

SEQ ID NO: 9 - RNA, 21 nt, synthetic construct
  misc_feature 1..21: note = artificial sequence
  source 1..21: mol_type = other RNA
cttcatccagacagacaagac

SEQ ID NO: 10 - RNA, 21 nt, synthetic construct
  misc_feature 1..21: note = artificial sequence
  source 1..21: mol_type = other RNA
catccagacagacaagaccat

SEQ ID NO: 11 - RNA, 21 nt, synthetic construct
  misc_feature 1..21: note = artificial sequence
  source 1..21: mol_type = other RNA
aacaccatgaggtcaaagggc

SEQ ID NO: 12 - RNA, 21 nt, synthetic construct
  misc_feature 1..21: note = artificial sequence
  source 1..21: mol_type = other RNA
acaccatgaggtcaaagggca

SEQ ID NO: 13 - RNA, 21 nt, synthetic construct
  misc_feature 1..21: note = artificial sequence
  source 1..21: mol_type = other RNA
ttgtctgtctggatgaagagg

SEQ ID NO: 14 - RNA, 21 nt, synthetic construct
  misc_feature 1..21: note = artificial sequence
  source 1..21: mol_type = other RNA
gaacaccatgaggtcaaaggg

SEQ ID NO: 15 - RNA, 21 nt, synthetic construct
  misc_feature 1..21: note = artificial sequence
  source 1..21: mol_type = other RNA
tgtctgtctggatgaagaggt

SEQ ID NO: 16 - RNA, 21 nt, synthetic construct
  misc_feature 1..21: note = artificial sequence
  source 1..21: mol_type = other RNA
tctggatgaagaggtacccgc

SEQ ID NO: 17 - RNA, 21 nt, synthetic construct
  misc_feature 1..21: note = artificial sequence
  source 1..21: mol_type = other RNA
gtgtagatggtcttgtctgtc

SEQ ID NO: 18 - RNA, 21 nt, synthetic construct
  misc_feature 1..21: note = artificial sequence
  source 1..21: mol_type = other RNA
agatggtcttgtctgtctgga

SEQ ID NO: 19 - RNA, 21 nt, synthetic construct
  misc_feature 1..21: note = artificial sequence
  source 1..21: mol_type = other RNA
gtcttgtctgtctggatgaag

SEQ ID NO: 20 - RNA, 21 nt, synthetic construct
  misc_feature 1..21: note = artificial sequence
  source 1..21: mol_type = other RNA
atggtcttgtctgtctggatg

SEQ ID NO: 21 - RNA, 21 nt, synthetic construct
  misc_feature 1..21: note = artificial sequence
  source 1..21: mol_type = other RNA
aaggaactgtttacaactata

SEQ ID NO: 22 - RNA, 21 nt, synthetic construct
  misc_feature 1..21: note = artificial sequence
  source 1..21: mol_type = other RNA
ctggtatatgtgttgctgata

SEQ ID NO: 23 - RNA, 21 nt, synthetic construct
  misc_feature 1..21: note = artificial sequence
  source 1..21: mol_type = other RNA
cttttcctgacttcaagattc

SEQ ID NO: 24 - RNA, 21 nt, synthetic construct
  misc_feature 1..21: note = artificial sequence
  source 1..21: mol_type = other RNA
ttgaaaggaactgtttacaac

SEQ ID NO: 25 - RNA, 21 nt, synthetic construct
  misc_feature 1..21: note = artificial sequence
  source 1..21: mol_type = other RNA
attgaaaggaactgtttacaa

SEQ ID NO: 26 - RNA, 21 nt, synthetic construct
  misc_feature 1..21: note = artificial sequence
  source 1..21: mol_type = other RNA
ttttcctgacttcaagattcc

SEQ ID NO: 27 - RNA, 21 nt, synthetic construct
  misc_feature 1..21: note = artificial sequence
  source 1..21: mol_type = other RNA
aactgtcttaactttcataga

SEQ ID NO: 28 - RNA, 21 nt, synthetic construct
  misc_feature 1..21: note = artificial sequence
  source 1..21: mol_type = other RNA
tatctcttttcctgacttcaa

SEQ ID NO: 29 - RNA, 21 nt, synthetic construct
  misc_feature 1..21: note = artificial sequence
  source 1..21: mol_type = other RNA
actggtatatgtgttgctgat

SEQ ID NO: 30 - RNA, 21 nt, synthetic construct
  misc_feature 1..21: note = artificial sequence
  source 1..21: mol_type = other RNA
tcttttcctgacttcaagatt

SEQ ID NO: 31 - RNA, 21 nt, synthetic construct
  misc_feature 1..21: note = artificial sequence
  source 1..21: mol_type = other RNA
tatagttgtaaacagttcctt

SEQ ID NO: 32 - RNA, 21 nt, synthetic construct
  misc_feature 1..21: note = artificial sequence
  source 1..21: mol_type = other RNA
tatcagcaacacatataccag

SEQ ID NO: 33 - RNA, 21 nt, synthetic construct
  misc_feature 1..21: note = artificial sequence
  source 1..21: mol_type = other RNA
gaatcttgaagtcaggaaaag

SEQ ID NO: 34 - RNA, 21 nt, synthetic construct
  misc_feature 1..21: note = artificial sequence
  source 1..21: mol_type = other RNA
gttgtaaacagttcctttcaa

SEQ ID NO: 35 - RNA, 21 nt, synthetic construct
  misc_feature 1..21: note = artificial sequence
  source 1..21: mol_type = other RNA
ttgtaaacagttcctttcaat

SEQ ID NO: 36 - RNA, 21 nt, synthetic construct
  misc_feature 1..21: note = artificial sequence
  source 1..21: mol_type = other RNA
ggaatcttgaagtcaggaaaa

SEQ ID NO: 37 - RNA, 21 nt, synthetic construct
  misc_feature 1..21: note = artificial sequence
  source 1..21: mol_type = other RNA
tctatgaaagttaagacagtt

SEQ ID NO: 38 - RNA, 21 nt, synthetic construct
  misc_feature 1..21: note = artificial sequence
  source 1..21: mol_type = other RNA
ttgaagtcaggaaaagagata

SEQ ID NO: 39 - RNA, 21 nt, synthetic construct
  misc_feature 1..21: note = artificial sequence
  source 1..21: mol_type = other RNA
atcagcaacacatataccagt

SEQ ID NO: 40 - RNA, 21 nt, synthetic construct
  misc_feature 1..21: note = artificial sequence
  source 1..21: mol_type = other RNA
aatcttgaagtcaggaaaaga

SEQ ID NO: 41 - RNA, 21 nt, synthetic construct
  misc_feature 1..21: note = artificial sequence
  source 1..21: mol_type = other RNA
acctacaaagctgtgattcag

SEQ ID NO: 42 - RNA, 21 nt, synthetic construct
  misc_feature 1..21: note = artificial sequence
  source 1..21: mol_type = other RNA
tgtgattcagtacagctgtga

SEQ ID NO: 43 - RNA, 21 nt, synthetic construct
  misc_feature 1..21: note = artificial sequence
  source 1..21: mol_type = other RNA
gtggtttgtgggaggaatagt

SEQ ID NO: 44 - RNA, 21 nt, synthetic construct
  misc_feature 1..21: note = artificial sequence
  source 1..21: mol_type = other RNA
gcgcctctacttcacccactt

SEQ ID NO: 45 - RNA, 21 nt, synthetic construct
  misc_feature 1..21: note = artificial sequence
  source 1..21: mol_type = other RNA
tgtggagtccttcgatgtgga

SEQ ID NO: 46 - RNA, 21 nt, synthetic construct
  misc_feature 1..21: note = artificial sequence
  source 1..21: mol_type = other RNA
gatggtaaatatgtgtgtgag

SEQ ID NO: 47 - RNA, 21 nt, synthetic construct
  misc_feature 1..21: note = artificial sequence
  source 1..21: mol_type = other RNA
cctacaaagctgtgattcagt

SEQ ID NO: 48 - RNA, 21 nt, synthetic construct
  misc_feature 1..21: note = artificial sequence
  source 1..21: mol_type = other RNA
attcagtacagctgtgaagag

SEQ ID NO: 49 - RNA, 21 nt, synthetic construct
  misc_feature 1..21: note = artificial sequence
  source 1..21: mol_type = other RNA
tggagtccttcgatgtggaga

SEQ ID NO: 50 - RNA, 21 nt, synthetic construct
  misc_feature 1..21: note = artificial sequence
  source 1..21: mol_type = other RNA
ctgtgattcagtacagctgtg

SEQ ID NO: 51 - RNA, 21 nt, synthetic construct
  misc_feature 1..21: note = artificial sequence
  source 1..21: mol_type = other RNA
ctgaatcacagctttgtaggt

SEQ ID NO: 52 - RNA, 21 nt, synthetic construct
  misc_feature 1..21: note = artificial sequence
  source 1..21: mol_type = other RNA
tcacagctgtactgaatcaca

SEQ ID NO: 53 - RNA, 21 nt, synthetic construct
  misc_feature 1..21: note = artificial sequence
  source 1..21: mol_type = other RNA
actattcctcccacaaaccac

SEQ ID NO: 54 - RNA, 21 nt, synthetic construct
  misc_feature 1..21: note = artificial sequence
  source 1..21: mol_type = other RNA
aagtgggtgaagtagaggcgc

SEQ ID NO: 55 - RNA, 21 nt, synthetic construct
  misc_feature 1..21: note = artificial sequence
  source 1..21: mol_type = other RNA
tccacatcgaaggactccaca

SEQ ID NO: 56 - RNA, 21 nt, synthetic construct
  misc_feature 1..21: note = artificial sequence
  source 1..21: mol_type = other RNA
ctcacacacatatttaccatc

SEQ ID NO: 57 - RNA, 21 nt, synthetic construct
  misc_feature 1..21: note = artificial sequence
  source 1..21: mol_type = other RNA
actgaatcacagctttgtagg

SEQ ID NO: 58 - RNA, 21 nt, synthetic construct
  misc_feature 1..21: note = artificial sequence
  source 1..21: mol_type = other RNA
ctcttcacagctgtactgaat

SEQ ID NO: 59 - RNA, 21 nt, synthetic construct
  misc_feature 1..21: note = artificial sequence
  source 1..21: mol_type = other RNA
tctccacatcgaaggactcca

SEQ ID NO: 60 - RNA, 21 nt, synthetic construct
  misc_feature 1..21: note = artificial sequence
  source 1..21: mol_type = other RNA
cacagctgtactgaatcacag

SEQ ID NO: 61 - RNA, 21 nt, synthetic construct
  misc_feature 1..21: note = artificial sequence
  source 1..21: mol_type = other RNA
gccctttgacctcatggtgtt

SEQ ID NO: 62 - RNA, 21 nt, synthetic construct
  misc_feature 1..21: note = sense strand
  source 1..21: mol_type = other RNA
gtcatcacactgaataccaat

SEQ ID NO: 63 - RNA, 21 nt, synthetic construct
  misc_feature 1..21: note = antisense strand
  source 1..21: mol_type = other RNA
attggtattcagtgtgatgac

SEQ ID NO: 64 - RNA, 21 nt, synthetic construct
  misc_feature 1..21: note = passenger
  source 1..21: mol_type = other RNA
gtcatcacactgaataccaat

SEQ ID NO: 65 - RNA, 21 nt, synthetic construct
  misc_feature 1..21: note = guide
  source 1..21: mol_type = other RNA
attggtattcagtgtgatgac

SEQ ID NO: 66 - DNA, 5231 bp, Homo sapiens
  source 1..5231: mol_type = genomic DNA
actcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacccccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtgagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcccccactccagataaagcttcagttatatctcacgtgtctggagttctttgccaagagggagaggctgaaatccccagccgcctcacctgcagctcagctccatcctacttgaaacctcacctgttcccaccgcattttctcctggcgttcgcctgctagtgtg

SEQ ID NO: 67 - DNA, 5464 bp, Homo sapiens
  source 1..5464: mol_type = other DNA
atatccgtggtttcctgctacctccaaccatgggccttttgggaatactttgttttttaatcttcctggggaaaacctggggacaggagcaaacatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacaatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcagtaaaagttagagtttattcgttgaatgacgacttgaagccagccaaaagagaaactgtcttaactttcatagatcctgaaggatcagaagttgacatggtagaagaaattgatcatattggaattatctcttttcctgacttcaagattccgtctaatcctagatatggtatgtggacgatcaaggctaaatataaagaggacttttcaacaactggaaccgcatattttgaagttaaagaatatgtcttgccacatttttctgtctcaatcgagccagaatataatttcattggttacaagaactttaagaattttgaaattactataaaagcaagatatttttataataaagtagtcactgaggctgacgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatatcccatcaaggtgcaggttaaagattcgcttgaccagttggtaggaggagtcccagtaacactgaatgcacaaacaattgatgtaaaccaagagacatctgacttggatccaagcaaaagtgtaacacgtgttgatgatggagtagcttcctttgtgcttaatctcccatctggagtgacggtgctggagtttaatgtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttgattttatccaagggcaaaattatccactttggcacgagggagaaattttcagatgcatcttatcaaagtataaacattccagtaacacagaacatggttccttcatcccgacttctggtctattacatcgtcacaggagaacagacagcagaattagtgtctgattcagtctggttaaatattgaagaaaaatgtggcaaccagctccaggttcatctgtctcctgatgcagatgcatattctccaggccaaactgtgtctcttaatatggcaactggaatggattcctgggtggcattagcagcagtggacagtgctgtgtatggagtccaaagaggagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattctcaggccaagaagaacgctgcaaaagaagatagaagaaatagctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctacacatgaagaccctgttaccagtaagcaagccagaaattcggagttattttccagaaagctggttgtgggaagttcatcttgttcccagaagaaaacagttgcagtttgccctacctgattctctaaccacctgggaaattcaaggcgttggcatttcaaacactggtatatgtgttgctgatactgtcaaggcaaaggtgttcaaagatgtcttcctggaaatgaatataccatattctgttgtacgaggagaacagatccaattgaaaggaactgtttacaactataggacttctgggatgcagttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtgccagaaggtgtcaaaagggaaagctattctggtgttactttggatcctaggggtatttatggtaccattagcagacgaaaggagttcccatacaggatacccttagatttggtccccaaaacagaaatcaaaaggattttgagtgtaaaaggactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaagggatgttgagcattatgtcctacagaaatgctgactactcttacagtgtgtggaagggtggaagtgctagcacttggttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacagggtaccttgcctgttgaagcccgagagaacagcttatatcttacagcctttactgtgattggaattagaaaggctttcgatatatgccccctggtgaaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaaggtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccaggacacaatcaatgccattgagggcctgacggaatattcactcctggttaaacaactccgcttgagtatggacatcgatgtttcttacaagcataaaggtgccttacataattataaaatgacagacaagaatttccttgggaggccagtagaggtgcttctcaatgatgacctcattgtcagtacaggatttggcagtggcttggctacagtacatgtaacaactgtagttcacaaaaccagtacctctgaggaagtttgcagcttttatttgaaaatcgatactcaggatattgaagcatcccactacagaggctacggaaactctgattacaaacgcatagtagcatgtgccagctacaagcccagcagggaagaatcatcatctggatcctctcatgcggtgatggacatctccttgcctactggaatcagtgcaaatgaagaagacttaaaagcccttgtggaaggggtggatcaactattcactgattaccaaatcaaagatggacatgttattctgcaactgaattcgattccctccagtgatttcctttgtgtacgattccggatatttgaactctttgaagttgggtttctcagtcctgccactttcacagtgtacgaataccacagaccagataaacagtgtaccatgttttatagcacttccaatatcaaaattcagaaagtctgtgaaggagccgcgtgcaagtgtgtagaagctgattgtgggcaaatgcaggaagaattggatctgacaatctctgcagagacaagaaaacaaacagcatgtaaaccagagattgcatatgcttataaagttagcatcacatccatcactgtagaaaatgtttttgtcaagtacaaggcaacccttctggatatctacaaaactggggaagctgttgctgagaaagactctgagattaccttcattaaaaaggtaacctgtactaacgctgagctggtaaaaggaagacagtacttaattatgggtaaagaagccctccagataaaatacaatttcagtttcaggtacatctaccctttagattccttgacctggattgaatactggcctagagacacaacatgttcatcgtgtcaagcatttttagctaatttagatgaatttgccgaagatatctttttaaatggatgctaaaattcctgaagttcagctgcatacagtttgcacttatggactcctgttgttgaagttcgtttttttgttttcttctttttttaaacattcatagctggtcttatttgtaaagctcactttacttagaattagtggcacttgcttttattagagaatgatttcaaatgctgtaactttctgaaataacatggccttggagggcatgaagacagatactcctccaaggttattggacaccggaaacaataaattggaacacctcctcaaacctaccactcaggaatgtttgctggggccgaaagaacagtccattgaaagggagtattacaaaaacatggcctttgcttgaaagaaaataccaaggaacaggaaactgatcattaaagcctgagtttgctttcaaa

SEQ ID NO: 68 - DNA, 2455 bp, Homo sapiens
  source 1..2455: mol_type = genomic DNA
agaccaggccaggccagctggacgggcacaccatgaggctgctgaccctcctgggccttctgtgtggctcggtggccacccccttgggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagccgaggacattgacgagtgccaggtggccccgggagaggcgcccacctgcgaccaccactgccacaaccacctgggcggtttctactgctcctgccgcgcaggctacgtcctgcaccgtaacaagcgcacctgctcagccctgtgctccggccaggtcttcacccagaggtctggggagctcagcagccctgaatacccacggccgtatcccaaactctccagttgcacttacagcatcagcctggaggaggggttcagtgtcattctggactttgtggagtccttcgatgtggagacacaccctgaaaccctgtgtccctacgactttctcaagattcaaacagacagagaagaacatggcccattctgtgggaagacattgccccacaggattgaaacaaaaagcaacacggtgaccatcacctttgtcacagatgaatcaggagaccacacaggctggaagatccactacacgagcacagcgcagccttgcccttatccgatggcgccacctaatggccacgtttcacctgtgcaagccaaatacatcctgaaagacagcttctccatcttttgcgagactggctatgagcttctgcaaggtcacttgcccctgaaatcctttactgcagtttgtcagaaagatggatcttgggaccggccaatgcccgcgtgcagcattgttgactgtggccctcctgatgatctacccagtggccgagtggagtacatcacaggtcctggagtgaccacctacaaagctgtgattcagtacagctgtgaagagaccttctacacaatgaaagtgaatgatggtaaatatgtgtgtgaggctgatggattctggacgagctccaaaggagaaaaatcactcccagtctgtgagcctgtttgtggactatcagcccgcacaacaggagggcgtatatatggagggcaaaaggcaaaacctggtgattttccttggcaagtcctgatattaggtggaaccacagcagcaggtgcacttttatatgacaactgggtcctaacagctgctcatgccgtctatgagcaaaaacatgatgcatccgccctggacattcgaatgggcaccctgaaaagactatcacctcattatacacaagcctggtctgaagctgtttttatacatgaaggttatactcatgatgctggctttgacaatgacatagcactgattaaattgaataacaaagttgtaatcaatagcaacatcacgcctatttgtctgccaagaaaagaagctgaatcctttatgaggacagatgacattggaactgcatctggatggggattaacccaaaggggttttcttgctagaaatctaatgtatgtcgacataccgattgttgaccatcaaaaatgtactgctgcatatgaaaagccaccctatccaaggggaagtgtaactgctaacatgctttgtgctggcttagaaagtgggggcaaggacagctgcagaggtgacagcggaggggcactggtgtttctagatagtgaaacagagaggtggtttgtgggaggaatagtgtcctggggttccatgaattgtggggaagcaggtcagtatggagtctacacaaaagttattaactatattccctggatcgagaacataattagtgatttttaacttgcgtgtctgcagtcaaggattcttcatttttagaaatgcctgtgaagaccttggcagcgacgtggctcgagaagcattcatcattactgtggacatggcagttgttgctccacccaaaaaaacagactccaggtgaggctgctgtcatttctccacttgccagtttaattccagccttacccattgactcaaggggacataaaccacgagagtgacagtcatctttgcccacccagtgtaatgtcactgctcaaattacatttcattaccttaaaaagccagtctcttttcatactggctgttggcatttctgtaaactgcctgtccatgctctttgtttttaaacttgttcttattga

SEQ ID NO: 69 - RNA, 19 nt, synthetic construct
  misc_feature 1..19: note = artificial sequence
  source 1..19: mol_type = other RNA
tcttggtgaagtggatctg

SEQ ID NO: 70 - RNA, 19 nt, synthetic construct
  misc_feature 1..19: note = artificial sequence
  source 1..19: mol_type = other RNA
tgagagaagaccttgacca

SEQ ID NO: 71 - RNA, 19 nt, synthetic construct
  misc_feature 1..19: note = artificial sequence
  source 1..19: mol_type = other RNA
agagagaagaccttgacca

SEQ ID NO: 72 - RNA, 19 nt, synthetic construct
  misc_feature 1..19: note = artificial sequence
  source 1..19: mol_type = other RNA
gttgtaataggcgtagacc

SEQ ID NO: 73 - RNA, 19 nt, synthetic construct
  misc_feature 1..19: note = artificial sequence
  source 1..19: mol_type = other RNA
attgtaataggcgtagacc

SEQ ID NO: 74 - RNA, 21 nt, synthetic construct
  misc_feature 1..21: note = artificial sequence
  source 1..21: mol_type = other RNA
ctctacgaagctcatgaatat

SEQ ID NO: 75 - RNA, 21 nt, synthetic construct
  misc_feature 1..21: note = artificial sequence
  source 1..21: mol_type = other RNA
ctgcaggaggctaaagatatt

SEQ ID NO: 76 - RNA, 21 nt, synthetic construct
  misc_feature 1..21: note = artificial sequence
  source 1..21: mol_type = other RNA
ctccactgagtttgaggtgaa

SEQ ID NO: 77 - RNA, 21 nt, synthetic construct
  misc_feature 1..21: note = artificial sequence
  source 1..21: mol_type = other RNA
gtccaatgactttgacgagta

SEQ ID NO: 78 - RNA, 21 nt, synthetic construct
  misc_feature 1..21: note = artificial sequence
  source 1..21: mol_type = other RNA
caggagtaacctggatgagga

SEQ ID NO: 79 - RNA, 19 nt, synthetic construct
  misc_feature 1..19: note = artificial sequence
  source 1..19: mol_type = other RNA
tcttggtgaagtggatctg

SEQ ID NO: 80 - RNA, 19 nt, synthetic construct
  misc_feature 1..19: note = artificial sequence
  source 1..19: mol_type = other RNA
tgagagaagaccttgacca

SEQ ID NO: 81 - RNA, 19 nt, synthetic construct
  misc_feature 1..19: note = artificial sequence
  source 1..19: mol_type = other RNA
agagagaagaccttgacca

SEQ ID NO: 82 - RNA, 19 nt, synthetic construct
  misc_feature 1..19: note = artificial sequence
  source 1..19: mol_type = other RNA
gttgtaataggcgtagacc

SEQ ID NO: 83 - RNA, 19 nt, synthetic construct
  misc_feature 1..19: note = artificial sequence
  source 1..19: mol_type = other RNA
attgtaataggcgtagacc

SEQ ID NO: 84 - RNA, 21 nt, synthetic construct
  misc_feature 1..21: note = artificial sequence
  source 1..21: mol_type = other RNA
ctctacgaagctcatgaatat

SEQ ID NO: 85 - RNA, 21 nt, synthetic construct
  misc_feature 1..21: note = artificial sequence
  source 1..21: mol_type = other RNA
ctgcaggaggctaaagatatt

SEQ ID NO: 86 - RNA, 21 nt, synthetic construct
  misc_feature 1..21: note = artificial sequence
  source 1..21: mol_type = other RNA
ctccactgagtttgaggtgaa

SEQ ID NO: 87 - RNA, 21 nt, synthetic construct
  misc_feature 1..21: note = artificial sequence
  source 1..21: mol_type = other RNA
gtccaatgactttgacgagta

SEQ ID NO: 88 - RNA, 21 nt, synthetic construct
  misc_feature 1..21: note = artificial sequence
  source 1..21: mol_type = other RNA
caggagtaacctggatgagga

SEQ ID NO: 89 - RNA, 19 nt, synthetic construct
  misc_feature 1..19: note = artificial sequence
  source 1..19: mol_type = other RNA
cagatccacttcaccaaga

SEQ ID NO: 90 - RNA, 19 nt, synthetic construct
  misc_feature 1..19: note = artificial sequence
  source 1..19: mol_type = other RNA
tggtcaaggtcttctctct

SEQ ID NO: 91 - RNA, 19 nt, synthetic construct
  misc_feature 1..19: note = artificial sequence
  source 1..19: mol_type = other RNA
tggtcaaggtcttctctct

SEQ ID NO: 92 - RNA, 19 nt, synthetic construct
  misc_feature 1..19: note = artificial sequence
  source 1..19: mol_type = other RNA
ggtctacgcctattacaac

SEQ ID NO: 93 - RNA, 19 nt, synthetic construct
  misc_feature 1..19: note = artificial sequence
  source 1..19: mol_type = other RNA
ggtctacgcctattacaat

SEQ ID NO: 94 - RNA, 21 nt, synthetic construct
  misc_feature 1..21: note = artificial sequence
  source 1..21: mol_type = other RNA
atattcatgagcttcgtagag

SEQ ID NO: 95 - RNA, 21 nt, synthetic construct
  misc_feature 1..21: note = artificial sequence
  source 1..21: mol_type = other RNA
aatatctttagcctcctgcag

SEQ ID NO: 96 - RNA, 21 nt, synthetic construct
  misc_feature 1..21: note = artificial sequence
  source 1..21: mol_type = other RNA
ttcacctcaaactcagtggag

SEQ ID NO: 97 - RNA, 21 nt, synthetic construct
  misc_feature 1..21: note = artificial sequence
  source 1..21: mol_type = other RNA
tactcgtcaaagtcattggac

SEQ ID NO: 98 - RNA, 21 nt, synthetic construct
  misc_feature 1..21: note = artificial sequence
  source 1..21: mol_type = other RNA
tcctcatccaggttactcctg

SEQ ID NO: 99 - RNA, 19 nt, synthetic construct
  misc_feature 1..19: note = artificial sequence
  source 1..19: mol_type = other RNA
cagatccacttcaccaaga

SEQ ID NO: 100 - RNA, 24 nt, synthetic construct
  misc_feature 1..24: note = artificial sequence
  source 1..24: mol_type = other RNA
tggtcaaggtcttctctcttcacc

SEQ ID NO: 101 - RNA, 24 nt, synthetic construct
  misc_feature 1..24: note = artificial sequence
  source 1..24: mol_type = other RNA
tggtcaaggtcttctctcttcacc

SEQ ID NO: 102 - RNA, 24 nt, synthetic construct
  misc_feature 1..24: note = artificial sequence
  source 1..24: mol_type = other RNA
ggtctacgcctattacaactcacc

SEQ ID NO: 103 - RNA, 19 nt, synthetic construct
  misc_feature 1..19: note = artificial sequence
  source 1..19: mol_type = other RNA
ggtctacgcctattacaat

SEQ ID NO: 104 - RNA, 21 nt, synthetic construct
  misc_feature 1..21: note = artificial sequence
  source 1..21: mol_type = other RNA
atattcatgagcttcgtagag

SEQ ID NO: 105 - RNA, 21 nt, synthetic construct
  misc_feature 1..21: note = artificial sequence
  source 1..21: mol_type = other RNA
aatatctttagcctcctgcag

SEQ ID NO: 106 - RNA, 21 nt, synthetic construct
  misc_feature 1..21: note = artificial sequence
  source 1..21: mol_type = other RNA
ttcacctcaaactcagtggag

SEQ ID NO: 107 - RNA, 21 nt, synthetic construct
  misc_feature 1..21: note = artificial sequence
  source 1..21: mol_type = other RNA
tactcgtcaaagtcattggac

SEQ ID NO: 108 - RNA, 21 nt, synthetic construct
  misc_feature 1..21: note = artificial sequence
  source 1..21: mol_type = other RNA
tcctcatccaggttactcctg

SEQ ID NO: 109 - RNA, 21 nt, synthetic construct
  misc_feature 1..21: note = artificial sequence
  source 1..21: mol_type = other RNA
aagcaagatatttttataata

SEQ ID NO: 110 - RNA, 21 nt, synthetic construct
  misc_feature 1..21: note = artificial sequence
  source 1..21: mol_type = other RNA
gacgatcaaggctaaatataa

SEQ ID NO: 111 - RNA, 21 nt, synthetic construct
  misc_feature 1..21: note = artificial sequence
  source 1..21: mol_type = other RNA
tcccatcaaggtgcaggttaa

SEQ ID NO: 112 - RNA, 21 nt, synthetic construct
  misc_feature 1..21: note = artificial sequence
  source 1..21: mol_type = other RNA
cagtctgaacttgaaagatat

SEQ ID NO: 113 - RNA, 21 nt, synthetic construct
  misc_feature 1..21: note = artificial sequence
  source 1..21: mol_type = other RNA
ttcatttatcctcagagaata

SEQ ID NO: 114 - RNA, 21 nt, synthetic construct
  misc_feature 1..21: note = artificial sequence
  source 1..21: mol_type = other RNA
gagcattatgtcctacagaaa

SEQ ID NO: 115 - RNA, 21 nt, synthetic construct
  misc_feature 1..21: note = artificial sequence
  source 1..21: mol_type = other RNA
gactgataaccataaggcttt

SEQ ID NO: 116 - RNA, 21 nt, synthetic construct
  misc_feature 1..21: note = artificial sequence
  source 1..21: mol_type = other RNA
ctgtcttaactttcatagatc

SEQ ID NO: 117 - RNA, 21 nt, synthetic construct
  misc_feature 1..21: note = artificial sequence
  source 1..21: mol_type = other RNA
aatgatgaaccttgtaaagaa

SEQ ID NO: 118 - RNA, 21 nt, synthetic construct
  misc_feature 1..21: note = artificial sequence
  source 1..21: mol_type = other RNA
ctcaatcgagccagaatataa

SEQ ID NO: 119 - RNA, 21 nt, synthetic construct
  misc_feature 1..21: note = artificial sequence
  source 1..21: mol_type = other RNA
aagcaagatatttttataata

SEQ ID NO: 120 - RNA, 21 nt, synthetic construct
  misc_feature 1..21: note = artificial sequence
  source 1..21: mol_type = other RNA
gacgatcaaggctaaatataa

SEQ ID NO: 121 - RNA, 21 nt, synthetic construct
  misc_feature 1..21: note = artificial sequence
  source 1..21: mol_type = other RNA
tcccatcaaggtgcaggttaa

SEQ ID NO: 122 - RNA, 21 nt, synthetic construct
  misc_feature 1..21: note = artificial sequence
  source 1..21: mol_type = other RNA
cagtctgaacttgaaagatat

SEQ ID NO: 123 - RNA, 21 nt, synthetic construct
  misc_feature 1..21: note = artificial sequence
  source 1..21: mol_type = other RNA
ttcatttatcctcagagaata

SEQ ID NO: 124 - RNA, 21 nt, synthetic construct
  misc_feature 1..21: note = artificial sequence
  source 1..21: mol_type = other RNA
gagcattatgtcctacagaaa

SEQ ID NO: 125 - RNA, 21 nt, synthetic construct
  misc_feature 1..21: note = artificial sequence
  source 1..21: mol_type = other RNA
gactgataaccataaggcttt

SEQ ID NO: 126 - RNA, 21 nt, synthetic construct
  misc_feature 1..21: note = artificial sequence
  source 1..21: mol_type = other RNA
ctgtcttaactttcatagatc

SEQ ID NO: 127 - RNA, 21 nt, synthetic construct
  misc_feature 1..21: note = artificial sequence
  source 1..21: mol_type = other RNA
aatgatgaaccttgtaaagaa

SEQ ID NO: 128 - RNA, 21 nt, synthetic construct
  misc_feature 1..21: note = artificial sequence
  source 1..21: mol_type = other RNA
ctcaatcgagccagaatataa

SEQ ID NO: 129 - RNA, 23 nt, synthetic construct
  misc_feature 1..23: note = artificial sequence
  source 1..23: mol_type = other RNA
tattataaaaatatcttgctttt

SEQ ID NO: 130 - RNA, 21 nt, synthetic construct
  misc_feature 1..21: note = artificial sequence
  source 1..21: mol_type = other RNA
ttatatttagccttgatcgtc

SEQ ID NO: 131 - RNA, 21 nt, synthetic construct
  misc_feature 1..21: note = artificial sequence
  source 1..21: mol_type = other RNA
ttaacctgcaccttgatggga

SEQ ID NO: 132 - RNA, 21 nt, synthetic construct
  misc_feature 1..21: note = artificial sequence
  source 1..21: mol_type = other RNA
atatctttcaagttcagactg

SEQ ID NO: 133 - RNA, 21 nt, synthetic construct
  misc_feature 1..21: note = artificial sequence
  source 1..21: mol_type = other RNA
tattctctgaggataaatgaa

SEQ ID NO: 134 - RNA, 21 nt, synthetic construct
  misc_feature 1..21: note = artificial sequence
  source 1..21: mol_type = other RNA
tttctgtaggacataatgctc

SEQ ID NO: 135 - RNA, 21 nt, synthetic construct
  misc_feature 1..21: note = artificial sequence
  source 1..21: mol_type = other RNA
aaagccttatggttatcagtc

SEQ ID NO: 136 - RNA, 21 nt, synthetic construct
  misc_feature 1..21: note = artificial sequence
  source 1..21: mol_type = other RNA
gatctatgaaagttaagacag

SEQ ID NO: 137 - RNA, 21 nt, synthetic construct
  misc_feature 1..21: note = artificial sequence
  source 1..21: mol_type = other RNA
ttctttacaaggttcatcatt

SEQ ID NO: 138 - RNA, 21 nt, synthetic construct
  misc_feature 1..21: note = artificial sequence
  source 1..21: mol_type = other RNA
ttatattctggctcgattgag

SEQ ID NO: 139 - RNA, 25 nt, synthetic construct
  misc_feature 1..25: note = artificial sequence
  source 1..25: mol_type = other RNA
tattataaaaatatcttgctttttt

SEQ ID NO: 140 - RNA, 21 nt, synthetic construct
  misc_feature 1..21: note = artificial sequence
  source 1..21: mol_type = other RNA
ttatatttagccttgatcgtc

SEQ ID NO: 141 - RNA, 21 nt, synthetic construct
  misc_feature 1..21: note = artificial sequence
  source 1..21: mol_type = other RNA
ttaacctgcaccttgatggga

SEQ ID NO: 142 - RNA, 21 nt, synthetic construct
  misc_feature 1..21: note = artificial sequence
  source 1..21: mol_type = other RNA
atatctttcaagttcagactg

SEQ ID NO: 143 - RNA, 22 nt, synthetic construct
  misc_feature 1..22: note = artificial sequence
  source 1..22: mol_type = other RNA
tattctctgaggataaatgaac

SEQ ID NO: 144 - RNA, 21 nt, synthetic construct
  misc_feature 1..21: note = artificial sequence
  source 1..21: mol_type = other RNA
tttctgtaggacataatgctc

SEQ ID NO: 145 - RNA, 21 nt, synthetic construct
  misc_feature 1..21: note = artificial sequence
  source 1..21: mol_type = other RNA
aaagccttatggttatcagtc

SEQ ID NO: 146 - RNA, 21 nt, synthetic construct
  misc_feature 1..21: note = artificial sequence
  source 1..21: mol_type = other RNA
gatctatgaaagttaagacag

SEQ ID NO: 147 - RNA, 21 nt, synthetic construct
  misc_feature 1..21: note = artificial sequence
  source 1..21: mol_type = other RNA
ttctttacaaggttcatcatt

SEQ ID NO: 148 - RNA, 21 nt, synthetic construct
  misc_feature 1..21: note = artificial sequence
  source 1..21: mol_type = other RNA
ttatattctggctcgattgag

SEQ ID NO: 149 - RNA, 21 nt, synthetic construct
  misc_feature 1..21: note = artificial sequence
  source 1..21: mol_type = other RNA
gtcatcacactgaataccaat

SEQ ID NO: 150 - DNA, 19 bp, synthetic construct
  misc_feature 1..19: note = single stranded DNA
  source 1..19: mol_type = other DNA
cgaagcgccctactccact

SEQ ID NO: 151 - RNA, 21 nt, synthetic construct
  misc_feature 1..21: note = artificial sequence
  source 1..21: mol_type = other RNA
aagcctgagtttgctttcaaa

SEQ ID NO: 152 - RNA, 21 nt, synthetic construct
  misc_feature 1..21: note = artificial sequence
  source 1..21: mol_type = other RNA
atggcctttgcttgaaagaaa

SEQ ID NO: 153 - RNA, 21 nt, synthetic construct
  misc_feature 1..21: note = artificial sequence
  source 1..21: mol_type = other RNA
aagcaagatatttttataata

SEQ ID NO: 154 - RNA, 21 nt, synthetic construct
  misc_feature 1..21: note = artificial sequence
  source 1..21: mol_type = other RNA
aaggaagacagtacttaatta

SEQ ID NO: 155 - RNA, 21 nt, synthetic construct
  misc_feature 1..21: note = artificial sequence
  source 1..21: mol_type = other RNA
ctggttaaatattgaagaaaa

SEQ ID NO: 156 - RNA, 21 nt, synthetic construct
  misc_feature 1..21: note = artificial sequence
  source 1..21: mol_type = other RNA
aaggtgccttacataattata

SEQ ID NO: 157 - RNA, 21 nt, synthetic construct
  misc_feature 1..21: note = artificial sequence
  source 1..21: mol_type = other RNA
ttggaaaagaaatcttagtaa

SEQ ID NO: 158 - RNA, 21 nt, synthetic construct
  misc_feature 1..21: note = artificial sequence
  source 1..21: mol_type = other RNA
aaggaactgtttacaactata

SEQ ID NO: 159 - RNA, 21 nt, synthetic construct
  misc_feature 1..21: note = artificial sequence
  source 1..21: mol_type = other RNA
ctggggacaggagcaaacata

SEQ ID NO: 160 - RNA, 21 nt, synthetic construct
  misc_feature 1..21: note = artificial sequence
  source 1..21: mol_type = other RNA
atgctgtaactttctgaaata

SEQ ID NO: 161 - RNA, 21 nt, synthetic construct
  misc_feature 1..21: note = artificial sequence
  source 1..21: mol_type = other RNA
aaccgcatattttgaagttaa

SEQ ID NO: 162 - RNA, 21 nt, synthetic construct
  misc_feature 1..21: note = artificial sequence
  source 1..21: mol_type = other RNA
ctgggatgcagttctgtgtta

SEQ ID NO: 163 - RNA, 21 nt, synthetic construct
  misc_feature 1..21: note = artificial sequence
  source 1..21: mol_type = other RNA
caggaaatcattggaacattt

SEQ ID NO: 164 - RNA, 21 nt, synthetic construct
  misc_feature 1..21: note = artificial sequence
  source 1..21: mol_type = other RNA
ctggagacttggtttggaaaa

SEQ ID NO: 165 - RNA, 21 nt, synthetic construct
  misc_feature 1..21: note = artificial sequence
  source 1..21: mol_type = other RNA
atggaggtggcttttattcaa

SEQ ID NO: 166 - RNA, 21 nt, synthetic construct
  misc_feature 1..21: note = artificial sequence
  source 1..21: mol_type = other RNA
ttgaaagaaaataccaaggaa

SEQ ID NO: 167 - RNA, 21 nt, synthetic construct
  misc_feature 1..21: note = artificial sequence
  source 1..21: mol_type = other RNA
ttgggaatactttgtttttta

SEQ ID NO: 168 - RNA, 21 nt, synthetic construct
  misc_feature 1..21: note = artificial sequence
  source 1..21: mol_type = other RNA
caggccaaactgtgtctctta

SEQ ID NO: 169 - RNA, 21 nt, synthetic construct
  misc_feature 1..21: note = artificial sequence
  source 1..21: mol_type = other RNA
aaggaacaggaaactgatcat

SEQ ID NO: 170 - RNA, 21 nt, synthetic construct
  misc_feature 1..21: note = artificial sequence
  source 1..21: mol_type = other RNA
ttgaaagggagtattacaaaa

SEQ ID NO: 171 - RNA, 21 nt, synthetic construct
  misc_feature 1..21: note = artificial sequence
  source 1..21: mol_type = other RNA
cagaaactgaagaaaaaatta

SEQ ID NO: 172 - RNA, 21 nt, synthetic construct
  misc_feature 1..21: note = artificial sequence
  source 1..21: mol_type = other RNA
aagaagaacgctgcaaaagaa

SEQ ID NO: 173 - RNA, 21 nt, synthetic construct
  misc_feature 1..21: note = artificial sequence
  source 1..21: mol_type = other RNA
cacatccatcactgtagaaaa

SEQ ID NO: 174 - RNA, 21 nt, synthetic construct
  misc_feature 1..21: note = artificial sequence
  source 1..21: mol_type = other RNA
cagatgcatcttatcaaagta

SEQ ID NO: 175 - RNA, 21 nt, synthetic construct
  misc_feature 1..21: note = artificial sequence
  source 1..21: mol_type = other RNA
cactggagacttggtttggaa

SEQ ID NO: 176 - RNA, 21 nt, synthetic construct
  misc_feature 1..21: note = artificial sequence
  source 1..21: mol_type = other RNA
accgcatattttgaagttaaa

SEQ ID NO: 177 - RNA, 21 nt, synthetic construct
  misc_feature 1..21: note = artificial sequence
  source 1..21: mol_type = other RNA
ctgattcagtctggttaaata

SEQ ID NO: 178 - RNA, 21 nt, synthetic construct
  misc_feature 1..21: note = artificial sequence
  source 1..21: mol_type = other RNA
tacggaaactctgattacaaa

SEQ ID NO: 179 - RNA, 21 nt, synthetic construct
  misc_feature 1..21: note = artificial sequence
  source 1..21: mol_type = other RNA
ttgcttgaaagaaaataccaa

SEQ ID NO: 180 - RNA, 21 nt, synthetic construct
  misc_feature 1..21: note = artificial sequence
  source 1..21: mol_type = other RNA
ttccaaaactctgcaatctta

SEQ ID NO: 181 - RNA, 21 nt, synthetic construct
  misc_feature 1..21: note = artificial sequence
  source 1..21: mol_type = other RNA
aagagtacttggacaagtaaa

SEQ ID NO: 182 - RNA, 21 nt, synthetic construct
  misc_feature 1..21: note = artificial sequence
  source 1..21: mol_type = other RNA
ctgcaaaagaagatagaagaa

SEQ ID NO: 183 - RNA, 21 nt, synthetic construct
  misc_feature 1..21: note = artificial sequence
  source 1..21: mol_type = other RNA
ctgcagagacaagaaaacaaa

SEQ ID NO: 184 - RNA, 21 nt, synthetic construct
  misc_feature 1..21: note = artificial sequence
  source 1..21: mol_type = other RNA
aaccaataaaattacagggta

SEQ ID NO: 185 - RNA, 21 nt, synthetic construct
  misc_feature 1..21: note = artificial sequence
  source 1..21: mol_type = other RNA
ctggacttaccttcctcacta

SEQ ID NO: 186 - RNA, 21 nt, synthetic construct
  misc_feature 1..21: note = artificial sequence
  source 1..21: mol_type = other RNA
atggtaccattagcagacgaa

SEQ ID NO: 187 - RNA, 21 nt, synthetic construct
  misc_feature 1..21: note = artificial sequence
  source 1..21: mol_type = other RNA
tcgatgtttcttacaagcata

SEQ ID NO: 188 - RNA, 21 nt, synthetic construct
  misc_feature 1..21: note = artificial sequence
  source 1..21: mol_type = other RNA
caggaagaattggatctgaca

SEQ ID NO: 189 - RNA, 21 nt, synthetic construct
  misc_feature 1..21: note = artificial sequence
  source 1..21: mol_type = other RNA
aaagcctgagtttgctttcaa

SEQ ID NO: 190 - RNA, 21 nt, synthetic construct
  misc_feature 1..21: note = artificial sequence
  source 1..21: mol_type = other RNA
atgatgaaccttgtaaagaaa

SEQ ID NO: 191 - RNA, 21 nt, synthetic construct
  misc_feature 1..21: note = artificial sequence
  source 1..21: mol_type = other RNA
ttggacaccggaaacaataaa

SEQ ID NO: 192 - RNA, 21 nt, synthetic construct
  misc_feature 1..21: note = artificial sequence
  source 1..21: mol_type = other RNA
aagaaatagctgctaaatata

SEQ ID NO: 193 - RNA, 21 nt, synthetic construct
  misc_feature 1..21: note = artificial sequence
  source 1..21: mol_type = other RNA
aaggttattggacaccggaaa

SEQ ID NO: 194 - RNA, 21 nt, synthetic construct
  misc_feature 1..21: note = artificial sequence
  source 1..21: mol_type = other RNA
ctgaagaaaaaattaaaagaa

SEQ ID NO: 195 - RNA, 21 nt, synthetic construct
  misc_feature 1..21: note = artificial sequence
  source 1..21: mol_type = other RNA
tggcctttgcttgaaagaaaa

SEQ ID NO: 196 - RNA, 21 nt, synthetic construct
  misc_feature 1..21: note = artificial sequence
  source 1..21: mol_type = other RNA
atgcaacaatctctattaaaa

SEQ ID NO: 197 - RNA, 21 nt, synthetic construct
  misc_feature 1..21: note = artificial sequence
  source 1..21: mol_type = other RNA
tgggaatactttgttttttaa

SEQ ID NO: 198 - RNA, 21 nt, synthetic construct
  misc_feature 1..21: note = artificial sequence
  source 1..21: mol_type = other RNA
aagcatttttcaaaatcaaaa

SEQ ID NO: 199 - RNA, 21 nt, synthetic construct
  misc_feature 1..21: note = artificial sequence
  source 1..21: mol_type = other RNA
atcgacacagctctaattaaa

SEQ ID NO: 200 - RNA, 21 nt, synthetic construct
  misc_feature 1..21: note = artificial sequence
  source 1..21: mol_type = other RNA
atggatttctcttcattcata

SEQ ID NO: 201 - RNA, 21 nt, synthetic construct
  misc_feature 1..21: note = artificial sequence
  source 1..21: mol_type = other RNA
aagaagggatgttgagcatta

SEQ ID NO: 202 - RNA, 21 nt, synthetic construct
  misc_feature 1..21: note = artificial sequence
  source 1..21: mol_type = other RNA
atctttttaaatggatgctaa

SEQ ID NO: 203 - RNA, 21 nt, synthetic construct
  misc_feature 1..21: note = artificial sequence
  source 1..21: mol_type = other RNA
cccatttatcgtttttggaaa

SEQ ID NO: 204 - RNA, 21 nt, synthetic construct
  misc_feature 1..21: note = artificial sequence
  source 1..21: mol_type = other RNA
ttgcatatgcttataaagtta

SEQ ID NO: 205 - RNA, 21 nt, synthetic construct
  misc_feature 1..21: note = artificial sequence
  source 1..21: mol_type = other RNA
aagaattttgaaattactata

SEQ ID NO: 206 - RNA, 21 nt, synthetic construct
  misc_feature 1..21: note = artificial sequence
  source 1..21: mol_type = other RNA
tcccatcaaggtgcaggttaa

SEQ ID NO: 207 - RNA, 21 nt, synthetic construct
  misc_feature 1..21: note = artificial sequence
  source 1..21: mol_type = other RNA
tggaaaagaaatcttagtaaa

SEQ ID NO: 208 - RNA, 21 nt, synthetic construct
  misc_feature 1..21: note = artificial sequence
  source 1..21: mol_type = other RNA
acagtacttaattatgggtaa

SEQ ID NO: 209 - RNA, 21 nt, synthetic construct
  misc_feature 1..21: note = artificial sequence
  source 1..21: mol_type = other RNA
aagggagtattacaaaaacat

SEQ ID NO: 210 - RNA, 21 nt, synthetic construct
  misc_feature 1..21: note = artificial sequence
  source 1..21: mol_type = other RNA
cagcctttactgtgattggaa

SEQ ID NO: 211 - RNA, 21 nt, synthetic construct
  misc_feature 1..21: note = artificial sequence
  source 1..21: mol_type = other RNA
acgctgagctggtaaaaggaa

SEQ ID NO: 212 - RNA, 21 nt, synthetic construct
  misc_feature 1..21: note = artificial sequence
  source 1..21: mol_type = other RNA
ttccaatatcaaaattcagaa

SEQ ID NO: 213 - RNA, 21 nt, synthetic construct
  misc_feature 1..21: note = artificial sequence
  source 1..21: mol_type = other RNA
gtggaaggggtggatcaacta

SEQ ID NO: 214 - RNA, 21 nt, synthetic construct
  misc_feature 1..21: note = artificial sequence
  source 1..21: mol_type = other RNA
ccagtctgaacttgaaagata

SEQ ID NO: 215 - RNA, 21 nt, synthetic construct
  misc_feature 1..21: note = artificial sequence
  source 1..21: mol_type = other RNA
aacaatctctattaaaagtta

SEQ ID NO: 216 - RNA, 21 nt, synthetic construct
  misc_feature 1..21: note = artificial sequence
  source 1..21: mol_type = other RNA
ctgagattaccttcattaaaa

SEQ ID NO: 217 - RNA, 21 nt, synthetic construct
  misc_feature 1..21: note = artificial sequence
  source 1..21: mol_type = other RNA
ttgccgaagatatctttttaa

SEQ ID NO: 218 - RNA, 21 nt, synthetic construct
  misc_feature 1..21: note = artificial sequence
  source 1..21: mol_type = other RNA
accggaaacaataaattggaa

SEQ ID NO: 219 - RNA, 21 nt, synthetic construct
  misc_feature 1..21: note = artificial sequence
  source 1..21: mol_type = other RNA
aagcagaaactgaagaaaaaa

SEQ ID NO: 220 - RNA, 21 nt, synthetic construct
  misc_feature 1..21: note = artificial sequence
  source 1..21: mol_type = other RNA
aacaggaaactgatcattaaa

SEQ ID NO: 221 - RNA, 21 nt, synthetic construct
  misc_feature 1..21: note = artificial sequence
  source 1..21: mol_type = other RNA
aaagcagaaactgaagaaaaa

SEQ ID NO: 222 - RNA, 21 nt, synthetic construct
  misc_feature 1..21: note = artificial sequence
  source 1..21: mol_type = other RNA
ttggaacatttttcattctga

SEQ ID NO: 223 - RNA, 21 nt, synthetic construct
  misc_feature 1..21: note = artificial sequence
  source 1..21: mol_type = other RNA
aagaaattgatcatattggaa

SEQ ID NO: 224 - RNA, 21 nt, synthetic construct
  misc_feature 1..21: note = artificial sequence
  source 1..21: mol_type = other RNA
ctcttcattcatacagacaaa

SEQ ID NO: 225 - RNA, 21 nt, synthetic construct
  misc_feature 1..21: note = artificial sequence
  source 1..21: mol_type = other RNA
tgccgaagatatctttttaaa

SEQ ID NO: 226 - RNA, 21 nt, synthetic construct
  misc_feature 1..21: note = artificial sequence
  source 1..21: mol_type = other RNA
atcgatactcaggatattgaa

SEQ ID NO: 227 - RNA, 21 nt, synthetic construct
  misc_feature 1..21: note = artificial sequence
  source 1..21: mol_type = other RNA
cagtctgaacttgaaagatat

SEQ ID NO: 228 - RNA, 21 nt, synthetic construct
  misc_feature 1..21: note = artificial sequence
  source 1..21: mol_type = other RNA
aagaaaacaaacagcatgtaa

SEQ ID NO: 229 - RNA, 21 nt, synthetic construct
  misc_feature 1..21: note = artificial sequence
  source 1..21: mol_type = other RNA
caagtcacatttgattctgaa

SEQ ID NO: 230 - RNA, 21 nt, synthetic construct
  misc_feature 1..21: note = artificial sequence
  source 1..21: mol_type = other RNA
cagcttttatttgaaaatcga

SEQ ID NO: 231 - RNA, 21 nt, synthetic construct
  misc_feature 1..21: note = artificial sequence
  source 1..21: mol_type = other RNA
aagatatctttttaaatggat

SEQ ID NO: 232 - RNA, 21 nt, synthetic construct
  misc_feature 1..21: note = artificial sequence
  source 1..21: mol_type = other RNA
ctgaggaagtttgcagctttt

SEQ ID NO: 233 - RNA, 21 nt, synthetic construct
  misc_feature 1..21: note = artificial sequence
  source 1..21: mol_type = other RNA
cagtatcaaccaataaaatta

SEQ ID NO: 234 - RNA, 21 nt, synthetic construct
  misc_feature 1..21: note = artificial sequence
  source 1..21: mol_type = other RNA
atgcagatgactcccaagaaa

SEQ ID NO: 235 - RNA, 21 nt, synthetic construct
  misc_feature 1..21: note = artificial sequence
  source 1..21: mol_type = other RNA
ttggcatttcaaacactggta

SEQ ID NO: 236 - RNA, 21 nt, synthetic construct
  misc_feature 1..21: note = artificial sequence
  source 1..21: mol_type = other RNA
cacccacagtttcgttcaatt

SEQ ID NO: 237 - RNA, 21 nt, synthetic construct
  misc_feature 1..21: note = artificial sequence
  source 1..21: mol_type = other RNA
acccttctggatatctacaaa

SEQ ID NO: 238 - RNA, 21 nt, synthetic construct
  misc_feature 1..21: note = artificial sequence
  source 1..21: mol_type = other RNA
gagacttggtttggaaaagaa

SEQ ID NO: 239 - RNA, 21 nt, synthetic construct
  misc_feature 1..21: note = artificial sequence
  source 1..21: mol_type = other RNA
ctagttgagaattatcaatta

SEQ ID NO: 240 - RNA, 21 nt, synthetic construct
  misc_feature 1..21: note = artificial sequence
  source 1..21: mol_type = other RNA
cagggcacaaagtcctccaaa

SEQ ID NO: 241 - RNA, 21 nt, synthetic construct
  misc_feature 1..21: note = artificial sequence
  source 1..21: mol_type = other RNA
aagaactttaagaattttgaa

SEQ ID NO: 242 - RNA, 21 nt, synthetic construct
  misc_feature 1..21: note = artificial sequence
  source 1..21: mol_type = other RNA
ttggtttggaaaagaaatctt

SEQ ID NO: 243 - RNA, 21 nt, synthetic construct
  misc_feature 1..21: note = artificial sequence
  source 1..21: mol_type = other RNA
atggatgctaaaattcctgaa

SEQ ID NO: 244 - RNA, 21 nt, synthetic construct
  misc_feature 1..21: note = artificial sequence
  source 1..21: mol_type = other RNA
cagaaggtgtcaaaagggaaa

SEQ ID NO: 245 - RNA, 21 nt, synthetic construct
  misc_feature 1..21: note = artificial sequence
  source 1..21: mol_type = other RNA
ccgaaagaacagtccattgaa

SEQ ID NO: 246 - RNA, 21 nt, synthetic construct
  misc_feature 1..21: note = artificial sequence
  source 1..21: mol_type = other RNA
caaagcatttttcaaaatcaa

SEQ ID NO: 247 - RNA, 21 nt, synthetic construct
  misc_feature 1..21: note = artificial sequence
  source 1..21: mol_type = other RNA
ttggaagttgtatcaaagcat

SEQ ID NO: 248 - RNA, 21 nt, synthetic construct
  misc_feature 1..21: note = artificial sequence
  source 1..21: mol_type = other RNA
taaaagcaagatatttttata

SEQ ID NO: 249 - RNA, 21 nt, synthetic construct
  misc_feature 1..21: note = artificial sequence
  source 1..21: mol_type = other RNA
cagtacttaattatgggtaaa

SEQ ID NO: 250 - RNA, 21 nt, synthetic construct
  misc_feature 1..21: note = artificial sequence
  source 1..21: mol_type = other RNA
caggagcaaacatatgtcatt

SEQ ID NO: 251 - RNA, 21 nt, synthetic construct
  misc_feature 1..21: note = artificial sequence
  source 1..21: mol_type = other RNA
tagcacttggttaacagcttt

SEQ ID NO: 252 - RNA, 21 nt, synthetic construct
  misc_feature 1..21: note = artificial sequence
  source 1..21: mol_type = other RNA
tactgtgattggaattagaaa

SEQ ID NO: 253 - RNA, 21 nt, synthetic construct
  misc_feature 1..21: note = artificial sequence
  source 1..21: mol_type = other RNA
taccaaggaacaggaaactga

SEQ ID NO: 254 - RNA, 21 nt, synthetic construct
  misc_feature 1..21: note = artificial sequence
  source 1..21: mol_type = other RNA
tgggagaacatctgaatatta

SEQ ID NO: 255 - RNA, 21 nt, synthetic construct
  misc_feature 1..21: note = artificial sequence
  source 1..21: mol_type = other RNA
cagaggtatggaggtggcttt

SEQ ID NO: 256 - RNA, 21 nt, synthetic construct
  misc_feature 1..21: note = artificial sequence
  source 1..21: mol_type = other RNA
gtgctggagtttaatgtcaaa

SEQ ID NO: 257 - RNA, 21 nt, synthetic construct
  misc_feature 1..21: note = artificial sequence
  source 1..21: mol_type = other RNA
tccgtgttggagcatctgaaa

SEQ ID NO: 258 - RNA, 21 nt, synthetic construct
  misc_feature 1..21: note = artificial sequence
  source 1..21: mol_type = other RNA
aaagcatttttcaaaatcaaa

SEQ ID NO: 259 - RNA, 21 nt, synthetic construct
  misc_feature 1..21: note = artificial sequence
  source 1..21: mol_type = other RNA
aatgatgaaccttgtaaagaa

SEQ ID NO: 260 - RNA, 21 nt, synthetic construct
  misc_feature 1..21: note = artificial sequence
  source 1..21: mol_type = other RNA
ttggaataagagaagacttaa

SEQ ID NO: 261 - RNA, 21 nt, synthetic construct
  misc_feature 1..21: note = artificial sequence
  source 1..21: mol_type = other RNA
taccttcctcactaatgcaaa

SEQ ID NO: 262 - RNA, 21 nt, synthetic construct
  misc_feature 1..21: note = artificial sequence
  source 1..21: mol_type = other RNA
aaggaaaattcacagtatcaa

SEQ ID NO: 263 - RNA, 21 nt, synthetic construct
  misc_feature 1..21: note = artificial sequence
  source 1..21: mol_type = other RNA
atgcacaaacaattgatgtaa

SEQ ID NO: 264 - RNA, 21 nt, synthetic construct
  misc_feature 1..21: note = artificial sequence
  source 1..21: mol_type = other RNA
atgcatcttatcaaagtataa

SEQ ID NO: 265 - RNA, 21 nt, synthetic construct
  misc_feature 1..21: note = artificial sequence
  source 1..21: mol_type = other RNA
ctcacccacagtttcgttcaa

SEQ ID NO: 266 - RNA, 21 nt, synthetic construct
  misc_feature 1..21: note = artificial sequence
  source 1..21: mol_type = other RNA
atgggccttttgggaatactt

SEQ ID NO: 267 - RNA, 21 nt, synthetic construct
  misc_feature 1..21: note = artificial sequence
  source 1..21: mol_type = other RNA
tgggatgcagttctgtgttaa

SEQ ID NO: 268 - RNA, 21 nt, synthetic construct
  misc_feature 1..21: note = artificial sequence
  source 1..21: mol_type = other RNA
aagtacctttatattgctgta

SEQ ID NO: 269 - RNA, 21 nt, synthetic construct
  misc_feature 1..21: note = artificial sequence
  source 1..21: mol_type = other RNA
tggctagttgagaattatcaa

SEQ ID NO: 270 - RNA, 21 nt, synthetic construct
  misc_feature 1..21: note = artificial sequence
  source 1..21: mol_type = other RNA
gtggacgatcaaggctaaata

SEQ ID NO: 271 - RNA, 21 nt, synthetic construct
  misc_feature 1..21: note = artificial sequence
  source 1..21: mol_type = other RNA
ttgctgatactgtcaaggcaa

SEQ ID NO: 272 - RNA, 21 nt, synthetic construct
  misc_feature 1..21: note = artificial sequence
  source 1..21: mol_type = other RNA
tagctgctaaatataaacatt

SEQ ID NO: 273 - RNA, 21 nt, synthetic construct
  misc_feature 1..21: note = artificial sequence
  source 1..21: mol_type = other RNA
aagacttaaaagatgatcaaa

SEQ ID NO: 274 - RNA, 21 nt, synthetic construct
  misc_feature 1..21: note = artificial sequence
  source 1..21: mol_type = other RNA
aacagatccaattgaaaggaa

SEQ ID NO: 275 - RNA, 21 nt, synthetic construct
  misc_feature 1..21: note = artificial sequence
  source 1..21: mol_type = other RNA
acgatcaaggctaaatataaa

SEQ ID NO: 276 - RNA, 21 nt, synthetic construct
  misc_feature 1..21: note = artificial sequence
  source 1..21: mol_type = other RNA
caaggttattggacaccggaa

SEQ ID NO: 277 - RNA, 21 nt, synthetic construct
  misc_feature 1..21: note = artificial sequence
  source 1..21: mol_type = other RNA
ctcaaacctaccactcaggaa

SEQ ID NO: 278 - RNA, 21 nt, synthetic construct
  misc_feature 1..21: note = artificial sequence
  source 1..21: mol_type = other RNA
agcaagatatttttataataa

SEQ ID NO: 279 - RNA, 21 nt, synthetic construct
  misc_feature 1..21: note = artificial sequence
  source 1..21: mol_type = other RNA
accaaggaacaggaaactgat

SEQ ID NO: 280 - RNA, 21 nt, synthetic construct
  misc_feature 1..21: note = artificial sequence
  source 1..21: mol_type = other RNA
cagaccagtcagtaaaagtta

SEQ ID NO: 281 - RNA, 21 nt, synthetic construct
  misc_feature 1..21: note = artificial sequence
  source 1..21: mol_type = other RNA
gagcgttgtcccagtattcta

SEQ ID NO: 282 - RNA, 21 nt, synthetic construct
  misc_feature 1..21: note = artificial sequence
  source 1..21: mol_type = other RNA
cagaatataatttcattggtt

SEQ ID NO: 283 - RNA, 21 nt, synthetic construct
  misc_feature 1..21: note = artificial sequence
  source 1..21: mol_type = other RNA
ctggtatatgtgttgctgata

SEQ ID NO: 284 - RNA, 21 nt, synthetic construct
  misc_feature 1..21: note = artificial sequence
  source 1..21: mol_type = other RNA
caggaaggcatcaatatccta

SEQ ID NO: 285 - RNA, 21 nt, synthetic construct
  misc_feature 1..21: note = artificial sequence
  source 1..21: mol_type = other RNA
aacgctgagctggtaaaagga

SEQ ID NO: 286 - RNA, 21 nt, synthetic construct
  misc_feature 1..21: note = artificial sequence
  source 1..21: mol_type = other RNA
ttccgtgttggagcatctgaa

SEQ ID NO: 287 - RNA, 21 nt, synthetic construct
  misc_feature 1..21: note = artificial sequence
  source 1..21: mol_type = other RNA
atcgatgtttcttacaagcat

SEQ ID NO: 288 - RNA, 21 nt, synthetic construct
  misc_feature 1..21: note = artificial sequence
  source 1..21: mol_type = other RNA
ctggaaatgaatataccatat

SEQ ID NO: 289 - RNA, 21 nt, synthetic construct
  misc_feature 1..21: note = artificial sequence
  source 1..21: mol_type = other RNA
cagagattgcatatgcttata

SEQ ID NO: 290 - RNA, 21 nt, synthetic construct
  misc_feature 1..21: note = artificial sequence
  source 1..21: mol_type = other RNA
tacttaattatgggtaaagaa

SEQ ID NO: 291 - RNA, 21 nt, synthetic construct
  misc_feature 1..21: note = artificial sequence
  source 1..21: mol_type = other RNA
tccgtgctaatatctctcata

SEQ ID NO: 292 - RNA, 21 nt, synthetic construct
  misc_feature 1..21: note = artificial sequence
  source 1..21: mol_type = other RNA
acccatttatcgtttttggaa

SEQ ID NO: 293 - RNA, 21 nt, synthetic construct
  misc_feature 1..21: note = artificial sequence
  source 1..21: mol_type = other RNA
ttggatccaagcaaaagtgta

SEQ ID NO: 294 - RNA, 21 nt, synthetic construct
  misc_feature 1..21: note = artificial sequence
  source 1..21: mol_type = other RNA
tagagcagaaccaaaattcaa

SEQ ID NO: 295 - RNA, 21 nt, synthetic construct
  misc_feature 1..21: note = artificial sequence
  source 1..21: mol_type = other RNA
cagcataaagacagctctgta

SEQ ID NO: 296 - RNA, 21 nt, synthetic construct
  misc_feature 1..21: note = artificial sequence
  source 1..21: mol_type = other RNA
ccgtgctaatatctctcataa

SEQ ID NO: 297 - RNA, 21 nt, synthetic construct
  misc_feature 1..21: note = artificial sequence
  source 1..21: mol_type = other RNA
atgatgcaaacagcaatgcaa

SEQ ID NO: 298 - RNA, 21 nt, synthetic construct
  misc_feature 1..21: note = artificial sequence
  source 1..21: mol_type = other RNA
ttgaaattactataaaagcaa

SEQ ID NO: 299 - RNA, 21 nt, synthetic construct
  misc_feature 1..21: note = artificial sequence
  source 1..21: mol_type = other RNA
taagagtacttggacaagtaa

SEQ ID NO: 300 - RNA, 21 nt, synthetic construct
  misc_feature 1..21: note = artificial sequence
  source 1..21: mol_type = other RNA
cagacgaaaggagttcccata

SEQ ID NO: 301 - RNA, 21 nt, synthetic construct
  misc_feature 1..21: note = artificial sequence
  source 1..21: mol_type = other RNA
cagtttagaagatttaaacaa

SEQ ID NO: 302 - RNA, 21 nt, synthetic construct
  misc_feature 1..21: note = artificial sequence
  source 1..21: mol_type = other RNA
aactattcactgattaccaaa

SEQ ID NO: 303 - RNA, 21 nt, synthetic construct
  misc_feature 1..21: note = artificial sequence
  source 1..21: mol_type = other RNA
gagcattatgtcctacagaaa

SEQ ID NO: 304 - RNA, 21 nt, synthetic construct
  misc_feature 1..21: note = artificial sequence
  source 1..21: mol_type = other RNA
aacagtgtaccatgttttata

SEQ ID NO: 305 - RNA, 21 nt, synthetic construct
  misc_feature 1..21: note = artificial sequence
  source 1..21: mol_type = other RNA
ttgtatcaaagcatttttcaa

SEQ ID NO: 306 - RNA, 21 nt, synthetic construct
  misc_feature 1..21: note = artificial sequence
  source 1..21: mol_type = other RNA
atagctggtcttatttgtaaa

SEQ ID NO: 307 - RNA, 21 nt, synthetic construct
  misc_feature 1..21: note = artificial sequence
  source 1..21: mol_type = other RNA
tagatgaatttgccgaagata

SEQ ID NO: 308 - RNA, 21 nt, synthetic construct
  misc_feature 1..21: note = artificial sequence
  source 1..21: mol_type = other RNA
cagcagaattagtgtctgatt

SEQ ID NO: 309 - RNA, 21 nt, synthetic construct
  misc_feature 1..21: note = artificial sequence
  source 1..21: mol_type = other RNA
aagaaatcttagtaaaaacat

SEQ ID NO: 310 - RNA, 21 nt, synthetic construct
  misc_feature 1..21: note = artificial sequence
  source 1..21: mol_type = other RNA
cacttccaatatcaaaattca

SEQ ID NO: 311 - RNA, 21 nt, synthetic construct
  misc_feature 1..21: note = artificial sequence
  source 1..21: mol_type = other RNA
ctggtgttactttggatccta

SEQ ID NO: 312 - RNA, 21 nt, synthetic construct
  misc_feature 1..21: note = artificial sequence
  source 1..21: mol_type = other RNA
agccctccagataaaatacaa

SEQ ID NO: 313 - RNA, 21 nt, synthetic construct
  misc_feature 1..21: note = artificial sequence
  source 1..21: mol_type = other RNA
gacagtacttaattatgggta

SEQ ID NO: 314 - RNA, 21 nt, synthetic construct
  misc_feature 1..21: note = artificial sequence
  source 1..21: mol_type = other RNA
aagggcaaaattatccacttt

SEQ ID NO: 315 - RNA, 21 nt, synthetic construct
  misc_feature 1..21: note = artificial sequence
  source 1..21: mol_type = other RNA
tactacagtttagaagattta

SEQ ID NO: 316 - RNA, 21 nt, synthetic construct
  misc_feature 1..21: note = artificial sequence
  source 1..21: mol_type = other RNA
caagcatttttagctaattta

SEQ ID NO: 317 - RNA, 21 nt, synthetic construct
  misc_feature 1..21: note = artificial sequence
  source 1..21: mol_type = other RNA
caagtttatggatacactgaa

SEQ ID NO: 318 - RNA, 21 nt, synthetic construct
  misc_feature 1..21: note = artificial sequence
  source 1..21: mol_type = other RNA
tacattggccatttctgcgta

SEQ ID NO: 319 - RNA, 21 nt, synthetic construct
  misc_feature 1..21: note = artificial sequence
  source 1..21: mol_type = other RNA
cagggtaccttgcctgttgaa

SEQ ID NO: 320 - RNA, 21 nt, synthetic construct
  misc_feature 1..21: note = artificial sequence
  source 1..21: mol_type = other RNA
tccccaaaacagaaatcaaaa

SEQ ID NO: 321 - RNA, 21 nt, synthetic construct
  misc_feature 1..21: note = artificial sequence
  source 1..21: mol_type = other RNA
cgtgtcaagcatttttagcta

SEQ ID NO: 322 - RNA, 21 nt, synthetic construct
  misc_feature 1..21: note = artificial sequence
  source 1..21: mol_type = other RNA
aagaaatgatgcaaacagcaa

SEQ ID NO: 323 - RNA, 21 nt, synthetic construct
  misc_feature 1..21: note = artificial sequence
  source 1..21: mol_type = other RNA
ttgttttcttctttttttaaa

SEQ ID NO: 324 - RNA, 21 nt, synthetic construct
  misc_feature 1..21: note = artificial sequence
  source 1..21: mol_type = other RNA
ttgagaattatcaattagata

SEQ ID NO: 325 - RNA, 21 nt, synthetic construct
  misc_feature 1..21: note = artificial sequence
  source 1..21: mol_type = other RNA
gaggctgacgtttatatcaca

SEQ ID NO: 326 - RNA, 21 nt, synthetic construct
  misc_feature 1..21: note = artificial sequence
  source 1..21: mol_type = other RNA
gagaagctttggttaaaggta

SEQ ID NO: 327 - RNA, 21 nt, synthetic construct
  misc_feature 1..21: note = artificial sequence
  source 1..21: mol_type = other RNA
ttgattggactgataaccata

SEQ ID NO: 328 - RNA, 21 nt, synthetic construct
  misc_feature 1..21: note = artificial sequence
  source 1..21: mol_type = other RNA
aaggctttgctagtgggagaa

SEQ ID NO: 329 - RNA, 21 nt, synthetic construct
  misc_feature 1..21: note = artificial sequence
  source 1..21: mol_type = other RNA
tagtgggagaacatctgaata

SEQ ID NO: 330 - RNA, 21 nt, synthetic construct
  misc_feature 1..21: note = artificial sequence
  source 1..21: mol_type = other RNA
atagaagaaatagctgctaaa

SEQ ID NO: 331 - RNA, 21 nt, synthetic construct
  misc_feature 1..21: note = artificial sequence
  source 1..21: mol_type = other RNA
ttggcacgagggagaaatttt

SEQ ID NO: 332 - RNA, 21 nt, synthetic construct
  misc_feature 1..21: note = artificial sequence
  source 1..21: mol_type = other RNA
acgctgcaaaagaagatagaa

SEQ ID NO: 333 - RNA, 21 nt, synthetic construct
  misc_feature 1..21: note = artificial sequence
  source 1..21: mol_type = other RNA
ggggaagctgttgctgagaaa

SEQ ID NO: 334 - RNA, 21 nt, synthetic construct
  misc_feature 1..21: note = artificial sequence
  source 1..21: mol_type = other RNA
gacgatcaaggctaaatataa

SEQ ID NO: 335 - RNA, 21 nt, synthetic construct
  misc_feature 1..21: note = artificial sequence
  source 1..21: mol_type = other RNA
gagattgcatatgcttataaa

SEQ ID NO: 336 - RNA, 21 nt, synthetic construct
  misc_feature 1..21: note = artificial sequence
  source 1..21: mol_type = other RNA
gagggaatctgcacttcggaa

SEQ ID NO: 337 - RNA, 21 nt, synthetic construct
  misc_feature 1..21: note = artificial sequence
  source 1..21: mol_type = other RNA
ctcaatcgagccagaatataa

SEQ ID NO: 338 - RNA, 21 nt, synthetic construct
  misc_feature 1..21: note = artificial sequence
  source 1..21: mol_type = other RNA
ttcggagttattttccagaaa

SEQ ID NO: 339 - RNA, 21 nt, synthetic construct
  misc_feature 1..21: note = artificial sequence
  source 1..21: mol_type = other RNA
aacccttctggatatctacaa

SEQ ID NO: 340 - RNA, 21 nt, synthetic construct
  misc_feature 1..21: note = artificial sequence
  source 1..21: mol_type = other RNA
caactattcactgattaccaa

SEQ ID NO: 341 - RNA, 21 nt, synthetic construct
  misc_feature 1..21: note = artificial sequence
  source 1..21: mol_type = other RNA
agggagaaattttcagatgca

SEQ ID NO: 342 - RNA, 21 nt, synthetic construct
  misc_feature 1..21: note = artificial sequence
  source 1..21: mol_type = other RNA
aaacagcaatgcaaaacacaa

SEQ ID NO: 343 - RNA, 21 nt, synthetic construct
  misc_feature 1..21: note = artificial sequence
  source 1..21: mol_type = other RNA
tatgtggacgatcaaggctaa

SEQ ID NO: 344 - RNA, 21 nt, synthetic construct
  misc_feature 1..21: note = artificial sequence
  source 1..21: mol_type = other RNA
tggggaagctgttgctgagaa

SEQ ID NO: 345 - RNA, 21 nt, synthetic construct
  misc_feature 1..21: note = artificial sequence
  source 1..21: mol_type = other RNA
cacaatgttgataaatggaat

SEQ ID NO: 346 - RNA, 21 nt, synthetic construct
  misc_feature 1..21: note = artificial sequence
  source 1..21: mol_type = other RNA
aatcttaacaatacaaccaaa

SEQ ID NO: 347 - RNA, 21 nt, synthetic construct
  misc_feature 1..21: note = artificial sequence
  source 1..21: mol_type = other RNA
gaggaagtttgcagcttttat

SEQ ID NO: 348 - RNA, 21 nt, synthetic construct
  misc_feature 1..21: note = artificial sequence
  source 1..21: mol_type = other RNA
aagagagaagctttggttaaa

SEQ ID NO: 349 - RNA, 21 nt, synthetic construct
  misc_feature 1..21: note = artificial sequence
  source 1..21: mol_type = other RNA
caggccaagaagaacgctgca

SEQ ID NO: 350 - RNA, 21 nt, synthetic construct
  misc_feature 1..21: note = artificial sequence
  source 1..21: mol_type = other RNA
gtccattgaaagggagtatta

SEQ ID NO: 351 - RNA, 21 nt, synthetic construct
  misc_feature 1..21: note = artificial sequence
  source 1..21: mol_type = other RNA
atggtagaagaaattgatcat

SEQ ID NO: 352 - RNA, 21 nt, synthetic construct
  misc_feature 1..21: note = artificial sequence
  source 1..21: mol_type = other RNA
atcacatttggaataagagaa

SEQ ID NO: 353 - RNA, 21 nt, synthetic construct
  misc_feature 1..21: note = artificial sequence
  source 1..21: mol_type = other RNA
caccggaaacaataaattgga

SEQ ID NO: 354 - RNA, 21 nt, synthetic construct
  misc_feature 1..21: note = artificial sequence
  source 1..21: mol_type = other RNA
aagcataaaggtgccttacat

SEQ ID NO: 355 - RNA, 21 nt, synthetic construct
  misc_feature 1..21: note = artificial sequence
  source 1..21: mol_type = other RNA
ctgagaaagactctgagatta

SEQ ID NO: 356 - RNA, 21 nt, synthetic construct
  misc_feature 1..21: note = artificial sequence
  source 1..21: mol_type = other RNA
gacttaaaagatgatcaaaaa

SEQ ID NO: 357 - RNA, 21 nt, synthetic construct
  misc_feature 1..21: note = artificial sequence
  source 1..21: mol_type = other RNA
acgatggagcctgcgttaata

SEQ ID NO: 358 - RNA, 21 nt, synthetic construct
  misc_feature 1..21: note = artificial sequence
  source 1..21: mol_type = other RNA
tggggacaggagcaaacatat

SEQ ID NO: 359 - RNA, 21 nt, synthetic construct
  misc_feature 1..21: note = artificial sequence
  source 1..21: mol_type = other RNA
gagatcttgtctgcagttcta

SEQ ID NO: 360 - RNA, 21 nt, synthetic construct
  misc_feature 1..21: note = artificial sequence
  source 1..21: mol_type = other RNA
aaggcaaaggtgttcaaagat

SEQ ID NO: 361 - RNA, 21 nt, synthetic construct
  misc_feature 1..21: note = artificial sequence
  source 1..21: mol_type = other RNA
ataatggatctttcaaggaaa

SEQ ID NO: 362 - RNA, 21 nt, synthetic construct
  misc_feature 1..21: note = artificial sequence
  source 1..21: mol_type = other RNA
ctgatgagcgttgtcccagta

SEQ ID NO: 363 - RNA, 21 nt, synthetic construct
  misc_feature 1..21: note = artificial sequence
  source 1..21: mol_type = other RNA
ttggaaagacaatcttcagca

SEQ ID NO: 364 - RNA, 21 nt, synthetic construct
  misc_feature 1..21: note = artificial sequence
  source 1..21: mol_type = other RNA
aacccagtcatcaaatggcta

SEQ ID NO: 365 - RNA, 21 nt, synthetic construct
  misc_feature 1..21: note = artificial sequence
  source 1..21: mol_type = other RNA
aaggggtggatcaactattca

SEQ ID NO: 366 - RNA, 21 nt, synthetic construct
  misc_feature 1..21: note = artificial sequence
  source 1..21: mol_type = other RNA
catgttcatcgtgtcaagcat

SEQ ID NO: 367 - RNA, 21 nt, synthetic construct
  misc_feature 1..21: note = artificial sequence
  source 1..21: mol_type = other RNA
ctcatctctcagccaaagtta

SEQ ID NO: 368 - RNA, 21 nt, synthetic construct
  misc_feature 1..21: note = artificial sequence
  source 1..21: mol_type = other RNA
ctgtaactttctgaaataaca

SEQ ID NO: 369 - RNA, 21 nt, synthetic construct
  misc_feature 1..21: note = artificial sequence
  source 1..21: mol_type = other RNA
aagctcactttacttagaatt

SEQ ID NO: 370 - RNA, 21 nt, synthetic construct
  misc_feature 1..21: note = artificial sequence
  source 1..21: mol_type = other RNA
caccagtctgaacttgaaaga

SEQ ID NO: 371 - RNA, 21 nt, synthetic construct
  misc_feature 1..21: note = artificial sequence
  source 1..21: mol_type = other RNA
ttcatcccgacttctggtcta

SEQ ID NO: 372 - RNA, 21 nt, synthetic construct
  misc_feature 1..21: note = artificial sequence
  source 1..21: mol_type = other RNA
ttgatgcaacaatctctatta

SEQ ID NO: 373 - RNA, 21 nt, synthetic construct
  misc_feature 1..21: note = artificial sequence
  source 1..21: mol_type = other RNA
ccggatatttgaactctttga

SEQ ID NO: 374 - RNA, 21 nt, synthetic construct
  misc_feature 1..21: note = artificial sequence
  source 1..21: mol_type = other RNA
taccagtaagcaagccagaaa

SEQ ID NO: 375 - RNA, 21 nt, synthetic construct
  misc_feature 1..21: note = artificial sequence
  source 1..21: mol_type = other RNA
ccagaaggtgtcaaaagggaa

SEQ ID NO: 376 - RNA, 21 nt, synthetic construct
  misc_feature 1..21: note = artificial sequence
  source 1..21: mol_type = other RNA
accagagattgcatatgctta

SEQ ID NO: 377 - RNA, 21 nt, synthetic construct
  misc_feature 1..21: note = artificial sequence
  source 1..21: mol_type = other RNA
tggtttggaaaagaaatctta

SEQ ID NO: 378 - RNA, 21 nt, synthetic construct
  misc_feature 1..21: note = artificial sequence
  source 1..21: mol_type = other RNA
aggtgtcaaaagggaaagcta

SEQ ID NO: 379 - RNA, 21 nt, synthetic construct
  misc_feature 1..21: note = artificial sequence
  source 1..21: mol_type = other RNA
tggttacaagaactttaagaa

SEQ ID NO: 380 - RNA, 21 nt, synthetic construct
  misc_feature 1..21: note = artificial sequence
  source 1..21: mol_type = other RNA
cacggattagtttagggccaa

SEQ ID NO: 381 - RNA, 21 nt, synthetic construct
  misc_feature 1..21: note = artificial sequence
  source 1..21: mol_type = other RNA
gagagaacagcttatatctta

SEQ ID NO: 382 - RNA, 21 nt, synthetic construct
  misc_feature 1..21: note = artificial sequence
  source 1..21: mol_type = other RNA
gaacaggaaactgatcattaa

SEQ ID NO: 383 - RNA, 21 nt, synthetic construct
  misc_feature 1..21: note = artificial sequence
  source 1..21: mol_type = other RNA
acagatactcctccaaggtta

SEQ ID NO: 384 - RNA, 21 nt, synthetic construct
  misc_feature 1..21: note = artificial sequence
  source 1..21: mol_type = other RNA
aaacatggcctttgcttgaaa

SEQ ID NO: 385 - RNA, 21 nt, synthetic construct
  misc_feature 1..21: note = artificial sequence
  source 1..21: mol_type = other RNA
cactttcacagtgtacgaata

SEQ ID NO: 386 - RNA, 21 nt, synthetic construct
  misc_feature 1..21: note = artificial sequence
  source 1..21: mol_type = other RNA
ttcactgattaccaaatcaaa

SEQ ID NO: 387 - RNA, 21 nt, synthetic construct
  misc_feature 1..21: note = artificial sequence
  source 1..21: mol_type = other RNA
ctctgagattaccttcattaa

SEQ ID NO: 388 - RNA, 21 nt, synthetic construct
  misc_feature 1..21: note = artificial sequence
  source 1..21: mol_type = other RNA
tagaagctgattgtgggcaaa

SEQ ID NO: 389 - RNA, 21 nt, synthetic construct
  misc_feature 1..21: note = artificial sequence
  source 1..21: mol_type = other RNA
taaaaggaagacagtacttaa

SEQ ID NO: 390 - RNA, 21 nt, synthetic construct
  misc_feature 1..21: note = artificial sequence
  source 1..21: mol_type = other RNA
aaaagttatcctgataaaaaa

SEQ ID NO: 391 - RNA, 21 nt, synthetic construct
  misc_feature 1..21: note = artificial sequence
  source 1..21: mol_type = other RNA
ctgacaactttctgcttgaaa

SEQ ID NO: 392 - RNA, 21 nt, synthetic construct
  misc_feature 1..21: note = artificial sequence
  source 1..21: mol_type = other RNA
cagaaatgctgactactctta

SEQ ID NO: 393 - RNA, 21 nt, synthetic construct
  misc_feature 1..21: note = artificial sequence
  source 1..21: mol_type = other RNA
aaggttaccgagcaatagcat

SEQ ID NO: 394 - RNA, 21 nt, synthetic construct
  misc_feature 1..21: note = artificial sequence
  source 1..21: mol_type = other RNA
tccagtgatttcctttgtgta

SEQ ID NO: 395 - RNA, 21 nt, synthetic construct
  misc_feature 1..21: note = artificial sequence
  source 1..21: mol_type = other RNA
aacttgaaagatataaattat

SEQ ID NO: 396 - RNA, 21 nt, synthetic construct
  misc_feature 1..21: note = artificial sequence
  source 1..21: mol_type = other RNA
ctgcatacagtttgcacttat

SEQ ID NO: 397 - RNA, 21 nt, synthetic construct
  misc_feature 1..21: note = artificial sequence
  source 1..21: mol_type = other RNA
gtggtgccagaaggtgtcaaa

SEQ ID NO: 398 - RNA, 21 nt, synthetic construct
  misc_feature 1..21: note = artificial sequence
  source 1..21: mol_type = other RNA
aaggagttcccatacaggata

SEQ ID NO: 399 - RNA, 21 nt, synthetic construct
  misc_feature 1..21: note = artificial sequence
  source 1..21: mol_type = other RNA
aaagcaagatatttttataat

SEQ ID NO: 400 - RNA, 21 nt, synthetic construct
  misc_feature 1..21: note = artificial sequence
  source 1..21: mol_type = other RNA
ttgccctacctgattctctaa

SEQ ID NO: 401 - RNA, 21 nt, synthetic construct
  misc_feature 1..21: note = artificial sequence
  source 1..21: mol_type = other RNA
aagtcacatttgattctgaaa

SEQ ID NO: 402 - RNA, 21 nt, synthetic construct
  misc_feature 1..21: note = artificial sequence
  source 1..21: mol_type = other RNA
aactctgattacaaacgcata

SEQ ID NO: 403 - RNA, 21 nt, synthetic construct
  misc_feature 1..21: note = artificial sequence
  source 1..21: mol_type = other RNA
gtgggagaacatctgaatatt

SEQ ID NO: 404 - RNA, 21 nt, synthetic construct
  misc_feature 1..21: note = artificial sequence
  source 1..21: mol_type = other RNA
aagggtggaagtgctagcact

SEQ ID NO: 405 - RNA, 21 nt, synthetic construct
  misc_feature 1..21: note = artificial sequence
  source 1..21: mol_type = other RNA
ttcactgtgcttcctctggaa

SEQ ID NO: 406 - RNA, 21 nt, synthetic construct
  misc_feature 1..21: note = artificial sequence
  source 1..21: mol_type = other RNA
tagcacttccaatatcaaaat

SEQ ID NO: 407 - RNA, 21 nt, synthetic construct
  misc_feature 1..21: note = artificial sequence
  source 1..21: mol_type = other RNA
aggaagaattggatctgacaa

SEQ ID NO: 408 - RNA, 21 nt, synthetic construct
  misc_feature 1..21: note = artificial sequence
  source 1..21: mol_type = other RNA
ttggttacaagaactttaaga

SEQ ID NO: 409 - RNA, 21 nt, synthetic construct
  misc_feature 1..21: note = artificial sequence
  source 1..21: mol_type = other RNA
attgaaagggagtattacaaa

SEQ ID NO: 410 - RNA, 21 nt, synthetic construct
  misc_feature 1..21: note = artificial sequence
  source 1..21: mol_type = other RNA
aagtttgcagcttttatttga

SEQ ID NO: 411 - RNA, 21 nt, synthetic construct
  misc_feature 1..21: note = artificial sequence
  source 1..21: mol_type = other RNA
aagagaaactgtcttaacttt

SEQ ID NO: 412 - RNA, 21 nt, synthetic construct
  misc_feature 1..21: note = artificial sequence
  source 1..21: mol_type = other RNA
ttgaaaatcgatactcaggat

SEQ ID NO: 413 - RNA, 21 nt, synthetic construct
  misc_feature 1..21: note = artificial sequence
  source 1..21: mol_type = other RNA
atcccgacttctggtctatta

SEQ ID NO: 414 - RNA, 21 nt, synthetic construct
  misc_feature 1..21: note = artificial sequence
  source 1..21: mol_type = other RNA
aagaactgtcatactacagtt

SEQ ID NO: 415 - RNA, 21 nt, synthetic construct
  misc_feature 1..21: note = artificial sequence
  source 1..21: mol_type = other RNA
aacatttttcattctgaccca

SEQ ID NO: 416 - RNA, 21 nt, synthetic construct
  misc_feature 1..21: note = artificial sequence
  source 1..21: mol_type = other RNA
aggagaacagatccaattgaa

SEQ ID NO: 417 - RNA, 21 nt, synthetic construct
  misc_feature 1..21: note = artificial sequence
  source 1..21: mol_type = other RNA
agggaatctgcacttcggaaa

SEQ ID NO: 418 - RNA, 21 nt, synthetic construct
  misc_feature 1..21: note = artificial sequence
  source 1..21: mol_type = other RNA
ctctgcaatcttaacaataca

SEQ ID NO: 419 - RNA, 21 nt, synthetic construct
  misc_feature 1..21: note = artificial sequence
  source 1..21: mol_type = other RNA
gatggacatgttattctgcaa

SEQ ID NO: 420 - RNA, 21 nt, synthetic construct
  misc_feature 1..21: note = artificial sequence
  source 1..21: mol_type = other RNA
tccaatatcaaaattcagaaa

SEQ ID NO: 421 - RNA, 21 nt, synthetic construct
  misc_feature 1..21: note = artificial sequence
  source 1..21: mol_type = other RNA
ttaaaagatgatcaaaaagaa

SEQ ID NO: 422 - RNA, 21 nt, synthetic construct
  misc_feature 1..21: note = artificial sequence
  source 1..21: mol_type = other RNA
aagagaagacttaaaagatga

SEQ ID NO: 423 - RNA, 21 nt, synthetic construct
  misc_feature 1..21: note = artificial sequence
  source 1..21: mol_type = other RNA
gtgccttacataattataaaa

SEQ ID NO: 424 - RNA, 21 nt, synthetic construct
  misc_feature 1..21: note = artificial sequence
  source 1..21: mol_type = other RNA
aaatattgtgattcaagttta

SEQ ID NO: 425 - RNA, 21 nt, synthetic construct
  misc_feature 1..21: note = artificial sequence
  source 1..21: mol_type = other RNA
aggggtatttatggtaccatt

SEQ ID NO: 426 - RNA, 21 nt, synthetic construct
  misc_feature 1..21: note = artificial sequence
  source 1..21: mol_type = other RNA
gtgtcaagcatttttagctaa

SEQ ID NO: 427 - RNA, 21 nt, synthetic construct
  misc_feature 1..21: note = artificial sequence
  source 1..21: mol_type = other RNA
tcgagccagaatataatttca

SEQ ID NO: 428 - RNA, 21 nt, synthetic construct
  misc_feature 1..21: note = artificial sequence
  source 1..21: mol_type = other RNA
tcctcagagaataaattccaa

SEQ ID NO: 429 - RNA, 21 nt, synthetic construct
  misc_feature 1..21: note = artificial sequence
  source 1..21: mol_type = other RNA
tagtggcacttgcttttatta

SEQ ID NO: 430 - RNA, 21 nt, synthetic construct
  misc_feature 1..21: note = artificial sequence
  source 1..21: mol_type = other RNA
ctcactataattacttgattt

SEQ ID NO: 431 - RNA, 21 nt, synthetic construct
  misc_feature 1..21: note = artificial sequence
  source 1..21: mol_type = other RNA
cagtgcaaatgaagaagactt

SEQ ID NO: 432 - RNA, 21 nt, synthetic construct
  misc_feature 1..21: note = artificial sequence
  source 1..21: mol_type = other RNA
aggccaaactgtgtctcttaa

SEQ ID NO: 433 - RNA, 21 nt, synthetic construct
  misc_feature 1..21: note = artificial sequence
  source 1..21: mol_type = other RNA
tttgccgaagatatcttttta

SEQ ID NO: 434 - RNA, 21 nt, synthetic construct
  misc_feature 1..21: note = artificial sequence
  source 1..21: mol_type = other RNA
attgtggctagttgagaatta

SEQ ID NO: 435 - RNA, 21 nt, synthetic construct
  misc_feature 1..21: note = artificial sequence
  source 1..21: mol_type = other RNA
cacttgcttttattagagaat

SEQ ID NO: 436 - RNA, 21 nt, synthetic construct
  misc_feature 1..21: note = artificial sequence
  source 1..21: mol_type = other RNA
gcctgcgttaataatgatgaa

SEQ ID NO: 437 - RNA, 21 nt, synthetic construct
  misc_feature 1..21: note = artificial sequence
  source 1..21: mol_type = other RNA
tacaagcataaaggtgcctta

SEQ ID NO: 438 - RNA, 21 nt, synthetic construct
  misc_feature 1..21: note = artificial sequence
  source 1..21: mol_type = other RNA
atctgaaaatattgtgattca

SEQ ID NO: 439 - RNA, 21 nt, synthetic construct
  misc_feature 1..21: note = artificial sequence
  source 1..21: mol_type = other RNA
cacagtttcgttcaattgttt

SEQ ID NO: 440 - RNA, 21 nt, synthetic construct
  misc_feature 1..21: note = artificial sequence
  source 1..21: mol_type = other RNA
ctggtcttatttgtaaagctc

SEQ ID NO: 441 - RNA, 21 nt, synthetic construct
  misc_feature 1..21: note = artificial sequence
  source 1..21: mol_type = other RNA
gtctggttaaatattgaagaa

SEQ ID NO: 442 - RNA, 21 nt, synthetic construct
  misc_feature 1..21: note = artificial sequence
  source 1..21: mol_type = other RNA
aacactgaatgcacaaacaat

SEQ ID NO: 443 - RNA, 21 nt, synthetic construct
  misc_feature 1..21: note = artificial sequence
  source 1..21: mol_type = other RNA
ctacggaaactctgattacaa

SEQ ID NO: 444 - RNA, 21 nt, synthetic construct
  misc_feature 1..21: note = artificial sequence
  source 1..21: mol_type = other RNA
ctccaaatgtgtgcgccagaa

SEQ ID NO: 445 - RNA, 21 nt, synthetic construct
  misc_feature 1..21: note = artificial sequence
  source 1..21: mol_type = other RNA
gcacttgcttttattagagaa

SEQ ID NO: 446 - RNA, 21 nt, synthetic construct
  misc_feature 1..21: note = artificial sequence
  source 1..21: mol_type = other RNA
ttcctgaagttcagctgcata

SEQ ID NO: 447 - RNA, 21 nt, synthetic construct
  misc_feature 1..21: note = artificial sequence
  source 1..21: mol_type = other RNA
atcctaggggtatttatggta

SEQ ID NO: 448 - RNA, 21 nt, synthetic construct
  misc_feature 1..21: note = artificial sequence
  source 1..21: mol_type = other RNA
caaatgctgtaactttctgaa

SEQ ID NO: 449 - RNA, 21 nt, synthetic construct
  misc_feature 1..21: note = artificial sequence
  source 1..21: mol_type = other RNA
ctgcttgaaaatacactgcca

SEQ ID NO: 450 - RNA, 21 nt, synthetic construct
  misc_feature 1..21: note = artificial sequence
  source 1..21: mol_type = other RNA
atgttcatttatcctcagaga

SEQ ID NO: 451 - RNA, 21 nt, synthetic construct
  misc_feature 1..21: note = artificial sequence
  source 1..21: mol_type = other RNA
aactctgcaatcttaacaata

SEQ ID NO: 452 - RNA, 21 nt, synthetic construct
  misc_feature 1..21: note = artificial sequence
  source 1..21: mol_type = other RNA
ttccggatatttgaactcttt

SEQ ID NO: 453 - RNA, 21 nt, synthetic construct
  misc_feature 1..21: note = artificial sequence
  source 1..21: mol_type = other RNA
tccaaaactctgcaatcttaa

SEQ ID NO: 454 - RNA, 21 nt, synthetic construct
  misc_feature 1..21: note = artificial sequence
  source 1..21: mol_type = other RNA
aggtgccttacataattataa

SEQ ID NO: 455 - RNA, 21 nt, synthetic construct
  misc_feature 1..21: note = artificial sequence
  source 1..21: mol_type = other RNA
cccactacagaggctacggaa

SEQ ID NO: 456 - RNA, 21 nt, synthetic construct
  misc_feature 1..21: note = artificial sequence
  source 1..21: mol_type = other RNA
taagtcaggaaggcatcaata

SEQ ID NO: 457 - RNA, 21 nt, synthetic construct
  misc_feature 1..21: note = artificial sequence
  source 1..21: mol_type = other RNA
tcctgataaaaaatttagtta

SEQ ID NO: 458 - RNA, 21 nt, synthetic construct
  misc_feature 1..21: note = artificial sequence
  source 1..21: mol_type = other RNA
tacctttatattgctgtaaca

SEQ ID NO: 459 - RNA, 21 nt, synthetic construct
  misc_feature 1..21: note = artificial sequence
  source 1..21: mol_type = other RNA
caggtacatctaccctttaga

SEQ ID NO: 460 - RNA, 21 nt, synthetic construct
  misc_feature 1..21: note = artificial sequence
  source 1..21: mol_type = other RNA
caggacacaatcaatgccatt

SEQ ID NO: 461 - RNA, 21 nt, synthetic construct
  misc_feature 1..21: note = artificial sequence
  source 1..21: mol_type = other RNA
caaggcaaaggtgttcaaaga

SEQ ID NO: 462 - RNA, 21 nt, synthetic construct
  misc_feature 1..21: note = artificial sequence
  source 1..21: mol_type = other RNA
ctcttaatatggcaactggaa

SEQ ID NO: 463 - RNA, 21 nt, synthetic construct
  misc_feature 1..21: note = artificial sequence
  source 1..21: mol_type = other RNA
ctgtcatactacagtttagaa

SEQ ID NO: 464 - RNA, 21 nt, synthetic construct
  misc_feature 1..21: note = artificial sequence
  source 1..21: mol_type = other RNA
tccagataaaatacaatttca

SEQ ID NO: 465 - RNA, 21 nt, synthetic construct
  misc_feature 1..21: note = artificial sequence
  source 1..21: mol_type = other RNA
cccttctggatatctacaaaa

SEQ ID NO: 466 - RNA, 21 nt, synthetic construct
  misc_feature 1..21: note = artificial sequence
  source 1..21: mol_type = other RNA
tcagtctggttaaatattgaa

SEQ ID NO: 467 - RNA, 21 nt, synthetic construct
  misc_feature 1..21: note = artificial sequence
  source 1..21: mol_type = other RNA
aaagggagtattacaaaaaca

SEQ ID NO: 468 - RNA, 21 nt, synthetic construct
  misc_feature 1..21: note = artificial sequence
  source 1..21: mol_type = other RNA
ttcagtctggttaaatattga

SEQ ID NO: 469 - RNA, 21 nt, synthetic construct
  misc_feature 1..21: note = artificial sequence
  source 1..21: mol_type = other RNA
gtggcacttgcttttattaga

SEQ ID NO: 470 - RNA, 21 nt, synthetic construct
  misc_feature 1..21: note = artificial sequence
  source 1..21: mol_type = other RNA
tacgtagagcagaaccaaaat

SEQ ID NO: 471 - RNA, 21 nt, synthetic construct
  misc_feature 1..21: note = artificial sequence
  source 1..21: mol_type = other RNA
ctgtgtctcttaatatggcaa

SEQ ID NO: 472 - RNA, 21 nt, synthetic construct
  misc_feature 1..21: note = artificial sequence
  source 1..21: mol_type = other RNA
cccttggaaagagtatttcaa

SEQ ID NO: 473 - RNA, 21 nt, synthetic construct
  misc_feature 1..21: note = artificial sequence
  source 1..21: mol_type = other RNA
ctcagagaataaattccaaaa

SEQ ID NO: 474 - RNA, 21 nt, synthetic construct
  misc_feature 1..21: note = artificial sequence
  source 1..21: mol_type = other RNA
tgcacaaacaattgatgtaaa

SEQ ID NO: 475 - RNA, 21 nt, synthetic construct
  misc_feature 1..21: note = artificial sequence
  source 1..21: mol_type = other RNA
cacgagggagaaattttcaga

SEQ ID NO: 476 - RNA, 21 nt, synthetic construct
  misc_feature 1..21: note = artificial sequence
  source 1..21: mol_type = other RNA
taggacttctgggatgcagtt

SEQ ID NO: 477 - RNA, 21 nt, synthetic construct
  misc_feature 1..21: note = artificial sequence
  source 1..21: mol_type = other RNA
cactataattacttgatttta

SEQ ID NO: 478 - RNA, 21 nt, synthetic construct
  misc_feature 1..21: note = artificial sequence
  source 1..21: mol_type = other RNA
ttgaagttcgtttttttgttt

SEQ ID NO: 479 - RNA, 21 nt, synthetic construct
  misc_feature 1..21: note = artificial sequence
  source 1..21: mol_type = other RNA
gtggatcaactattcactgat

SEQ ID NO: 480 - RNA, 21 nt, synthetic construct
  misc_feature 1..21: note = artificial sequence
  source 1..21: mol_type = other RNA
tggagggcatgaagacagata

SEQ ID NO: 481 - RNA, 21 nt, synthetic construct
  misc_feature 1..21: note = artificial sequence
  source 1..21: mol_type = other RNA
gtgaaaatcgacacagctcta

SEQ ID NO: 482 - RNA, 21 nt, synthetic construct
  misc_feature 1..21: note = artificial sequence
  source 1..21: mol_type = other RNA
tactaacgctgagctggtaaa

SEQ ID NO: 483 - RNA, 21 nt, synthetic construct
  misc_feature 1..21: note = artificial sequence
  source 1..21: mol_type = other RNA
gaagacttaaaagatgatcaa

SEQ ID NO: 484 - RNA, 21 nt, synthetic construct
  misc_feature 1..21: note = artificial sequence
  source 1..21: mol_type = other RNA
ctcaccagtctgaacttgaaa

SEQ ID NO: 485 - RNA, 21 nt, synthetic construct
  misc_feature 1..21: note = artificial sequence
  source 1..21: mol_type = other RNA
gaggtatggaggtggctttta

SEQ ID NO: 486 - RNA, 21 nt, synthetic construct
  misc_feature 1..21: note = artificial sequence
  source 1..21: mol_type = other RNA
aacgctgcaaaagaagataga

SEQ ID NO: 487 - RNA, 21 nt, synthetic construct
  misc_feature 1..21: note = artificial sequence
  source 1..21: mol_type = other RNA
tacgaggagaacagatccaat

SEQ ID NO: 488 - RNA, 21 nt, synthetic construct
  misc_feature 1..21: note = artificial sequence
  source 1..21: mol_type = other RNA
ctgatcattaaagcctgagtt

SEQ ID NO: 489 - RNA, 21 nt, synthetic construct
  misc_feature 1..21: note = artificial sequence
  source 1..21: mol_type = other RNA
ctgaacttgaaagatataaat

SEQ ID NO: 490 - RNA, 21 nt, synthetic construct
  misc_feature 1..21: note = artificial sequence
  source 1..21: mol_type = other RNA
atcaaaaagaaatgatgcaaa

SEQ ID NO: 491 - RNA, 21 nt, synthetic construct
  misc_feature 1..21: note = artificial sequence
  source 1..21: mol_type = other RNA
tggacttaccttcctcactaa

SEQ ID NO: 492 - RNA, 21 nt, synthetic construct
  misc_feature 1..21: note = artificial sequence
  source 1..21: mol_type = other RNA
ttggatcctaggggtatttat

SEQ ID NO: 493 - RNA, 21 nt, synthetic construct
  misc_feature 1..21: note = artificial sequence
  source 1..21: mol_type = other RNA
ttgataaatggaattgctcaa

SEQ ID NO: 494 - RNA, 21 nt, synthetic construct
  misc_feature 1..21: note = artificial sequence
  source 1..21: mol_type = other RNA
caaacaattgatgtaaaccaa

SEQ ID NO: 495 - RNA, 21 nt, synthetic construct
  misc_feature 1..21: note = artificial sequence
  source 1..21: mol_type = other RNA
ttaagagtacttggacaagta

SEQ ID NO: 496 - RNA, 21 nt, synthetic construct
  misc_feature 1..21: note = artificial sequence
  source 1..21: mol_type = other RNA
acccttagatttggtccccaa

SEQ ID NO: 497 - RNA, 21 nt, synthetic construct
  misc_feature 1..21: note = artificial sequence
  source 1..21: mol_type = other RNA
taccaaatcaaagatggacat

SEQ ID NO: 498 - RNA, 21 nt, synthetic construct
  misc_feature 1..21: note = artificial sequence
  source 1..21: mol_type = other RNA
tgagcattatgtcctacagaa

SEQ ID NO: 499 - RNA, 21 nt, synthetic construct
  misc_feature 1..21: note = artificial sequence
  source 1..21: mol_type = other RNA
ttgtcccagtattctatgttt

SEQ ID NO: 500 - RNA, 21 nt, synthetic construct
  misc_feature 1..21: note = artificial sequence
  source 1..21: mol_type = other RNA
tcgtttttttgttttcttctt

SEQ ID NO: 501 - RNA, 21 nt, synthetic construct
  misc_feature 1..21: note = artificial sequence
  source 1..21: mol_type = other RNA
tagaattagtggcacttgctt

SEQ ID NO: 502 - RNA, 21 nt, synthetic construct
  misc_feature 1..21: note = artificial sequence
  source 1..21: mol_type = other RNA
gccaagaagaacgctgcaaaa

SEQ ID NO: 503 - RNA, 21 nt, synthetic construct
  misc_feature 1..21: note = artificial sequence
  source 1..21: mol_type = other RNA
aaagacaatcttcagcataaa

SEQ ID NO: 504 - RNA, 21 nt, synthetic construct
  misc_feature 1..21: note = artificial sequence
  source 1..21: mol_type = other RNA
caagaagaacgctgcaaaaga

SEQ ID NO: 505 - RNA, 21 nt, synthetic construct
  misc_feature 1..21: note = artificial sequence
  source 1..21: mol_type = other RNA
tctgacttggatccaagcaaa

SEQ ID NO: 506 - RNA, 21 nt, synthetic construct
  misc_feature 1..21: note = artificial sequence
  source 1..21: mol_type = other RNA
gacatgcaattgggaaggcta

SEQ ID NO: 507 - RNA, 21 nt, synthetic construct
  misc_feature 1..21: note = artificial sequence
  source 1..21: mol_type = other RNA
ttgaagccagccaaaagagaa

SEQ ID NO: 508 - RNA, 21 nt, synthetic construct
  misc_feature 1..21: note = artificial sequence
  source 1..21: mol_type = other RNA
cagataaacagtgtaccatgt

SEQ ID NO: 509 - RNA, 21 nt, synthetic construct
  misc_feature 1..21: note = artificial sequence
  source 1..21: mol_type = other RNA
aaccaaaattcaatttgtaat

SEQ ID NO: 510 - RNA, 21 nt, synthetic construct
  misc_feature 1..21: note = artificial sequence
  source 1..21: mol_type = other RNA
cttgattttatccaagggcaa

SEQ ID NO: 511 - RNA, 21 nt, synthetic construct
  misc_feature 1..21: note = artificial sequence
  source 1..21: mol_type = other RNA
tagagaatgatttcaaatgct

SEQ ID NO: 512 - RNA, 21 nt, synthetic construct
  misc_feature 1..21: note = artificial sequence
  source 1..21: mol_type = other RNA
tcacagtatcaaccaataaaa

SEQ ID NO: 513 - RNA, 21 nt, synthetic construct
  misc_feature 1..21: note = artificial sequence
  source 1..21: mol_type = other RNA
ttcatttatcctcagagaata

SEQ ID NO: 514 - RNA, 21 nt, synthetic construct
  misc_feature 1..21: note = artificial sequence
  source 1..21: mol_type = other RNA
aaggagccgcgtgcaagtgtg

SEQ ID NO: 515 - RNA, 21 nt, synthetic construct
  misc_feature 1..21: note = artificial sequence
  source 1..21: mol_type = other RNA
ttgtgggcaaatgcaggaaga

SEQ ID NO: 516 - RNA, 21 nt, synthetic construct
  misc_feature 1..21: note = artificial sequence
  source 1..21: mol_type = other RNA
ttgcttttattagagaatgat

SEQ ID NO: 517 - RNA, 21 nt, synthetic construct
  misc_feature 1..21: note = artificial sequence
  source 1..21: mol_type = other RNA
tggttaaatattgaagaaaaa

SEQ ID NO: 518 - RNA, 21 nt, synthetic construct
  misc_feature 1..21: note = artificial sequence
  source 1..21: mol_type = other RNA
aagcccagtcattgatcatca

SEQ ID NO: 519 - RNA, 21 nt, synthetic construct
  misc_feature 1..21: note = artificial sequence
  source 1..21: mol_type = other RNA
atgccccctggtgaaaatcga

SEQ ID NO: 520 - RNA, 21 nt, synthetic construct
  misc_feature 1..21: note = artificial sequence
  source 1..21: mol_type = other RNA
aagccagaaattcggagttat

SEQ ID NO: 521 - RNA, 21 nt, synthetic construct
  misc_feature 1..21: note = artificial sequence
  source 1..21: mol_type = other RNA
ctagcacttggttaacagctt

SEQ ID NO: 522 - RNA, 21 nt, synthetic construct
  misc_feature 1..21: note = artificial sequence
  source 1..21: mol_type = other RNA
gccagtagaggtgcttctcaa

SEQ ID NO: 523 - RNA, 21 nt, synthetic construct
  misc_feature 1..21: note = artificial sequence
  source 1..21: mol_type = other RNA
ttattagagaatgatttcaaa

SEQ ID NO: 524 - RNA, 21 nt, synthetic construct
  misc_feature 1..21: note = artificial sequence
  source 1..21: mol_type = other RNA
aagctggttgtgggaagttca

SEQ ID NO: 525 - RNA, 21 nt, synthetic construct
  misc_feature 1..21: note = artificial sequence
  source 1..21: mol_type = other RNA
ccacagtttcgttcaattgtt

SEQ ID NO: 526 - RNA, 21 nt, synthetic construct
  misc_feature 1..21: note = artificial sequence
  source 1..21: mol_type = other RNA
ttcaaggaaaattcacagtat

SEQ ID NO: 527 - RNA, 21 nt, synthetic construct
  misc_feature 1..21: note = artificial sequence
  source 1..21: mol_type = other RNA
catggcctttgcttgaaagaa

SEQ ID NO: 528 - RNA, 21 nt, synthetic construct
  misc_feature 1..21: note = artificial sequence
  source 1..21: mol_type = other RNA
ttcgttcaattgtttcagctt

SEQ ID NO: 529 - RNA, 21 nt, synthetic construct
  misc_feature 1..21: note = artificial sequence
  source 1..21: mol_type = other RNA
cggcacgtatggtagaaacaa

SEQ ID NO: 530 - RNA, 21 nt, synthetic construct
  misc_feature 1..21: note = artificial sequence
  source 1..21: mol_type = other RNA
atcccatcaaggtgcaggtta

SEQ ID NO: 531 - RNA, 21 nt, synthetic construct
  misc_feature 1..21: note = artificial sequence
  source 1..21: mol_type = other RNA
aacattcagtagtgaagaaat

SEQ ID NO: 532 - RNA, 21 nt, synthetic construct
  misc_feature 1..21: note = artificial sequence
  source 1..21: mol_type = other RNA
catgttttatagcacttccaa

SEQ ID NO: 533 - RNA, 21 nt, synthetic construct
  misc_feature 1..21: note = artificial sequence
  source 1..21: mol_type = other RNA
actcaccagtctgaacttgaa

SEQ ID NO: 534 - RNA, 21 nt, synthetic construct
  misc_feature 1..21: note = artificial sequence
  source 1..21: mol_type = other RNA
catagctggtcttatttgtaa

SEQ ID NO: 535 - RNA, 21 nt, synthetic construct
  misc_feature 1..21: note = artificial sequence
  source 1..21: mol_type = other RNA
ctccagtcacttggtgacatt

SEQ ID NO: 536 - RNA, 21 nt, synthetic construct
  misc_feature 1..21: note = artificial sequence
  source 1..21: mol_type = other RNA
tttgcagcttttatttgaaaa

SEQ ID NO: 537 - RNA, 21 nt, synthetic construct
  misc_feature 1..21: note = artificial sequence
  source 1..21: mol_type = other RNA
ttcctcactaatgcaaatgca

SEQ ID NO: 538 - RNA, 21 nt, synthetic construct
  misc_feature 1..21: note = artificial sequence
  source 1..21: mol_type = other RNA
aaccagagattgcatatgctt

SEQ ID NO: 539 - RNA, 21 nt, synthetic construct
  misc_feature 1..21: note = artificial sequence
  source 1..21: mol_type = other RNA
cagtaacactgaatgcacaaa

SEQ ID NO: 540 - RNA, 21 nt, synthetic construct
  misc_feature 1..21: note = artificial sequence
  source 1..21: mol_type = other RNA
aagaaaatgatgaaccttgta

SEQ ID NO: 541 - RNA, 21 nt, synthetic construct
  misc_feature 1..21: note = artificial sequence
  source 1..21: mol_type = other RNA
ttctttttttaaacattcata

SEQ ID NO: 542 - RNA, 21 nt, synthetic construct
  misc_feature 1..21: note = artificial sequence
  source 1..21: mol_type = other RNA
acggcacgtatggtagaaaca

SEQ ID NO: 543 - RNA, 21 nt, synthetic construct
  misc_feature 1..21: note = artificial sequence
  source 1..21: mol_type = other RNA
aatgcacaaacaattgatgta

SEQ ID NO: 544 - RNA, 21 nt, synthetic construct
  misc_feature 1..21: note = artificial sequence
  source 1..21: mol_type = other RNA
ttcaaagatgtcttcctggaa

SEQ ID NO: 545 - RNA, 21 nt, synthetic construct
  misc_feature 1..21: note = artificial sequence
  source 1..21: mol_type = other RNA
cggaatattcactcctggtta

SEQ ID NO: 546 - RNA, 21 nt, synthetic construct
  misc_feature 1..21: note = artificial sequence
  source 1..21: mol_type = other RNA
gacgacttgaagccagccaaa

SEQ ID NO: 547 - RNA, 21 nt, synthetic construct
  misc_feature 1..21: note = artificial sequence
  source 1..21: mol_type = other RNA
agcctttactgtgattggaat

SEQ ID NO: 548 - RNA, 21 nt, synthetic construct
  misc_feature 1..21: note = artificial sequence
  source 1..21: mol_type = other RNA
gtagaagctgattgtgggcaa

SEQ ID NO: 549 - RNA, 21 nt, synthetic construct
  misc_feature 1..21: note = artificial sequence
  source 1..21: mol_type = other RNA
ctgaagcatttgatgcaacaa

SEQ ID NO: 550 - RNA, 21 nt, synthetic construct
  misc_feature 1..21: note = artificial sequence
  source 1..21: mol_type = other RNA
cagcagtcaaagaactgtcat

SEQ ID NO: 551 - RNA, 21 nt, synthetic construct
  misc_feature 1..21: note = artificial sequence
  source 1..21: mol_type = other RNA
ctgaatgcacaaacaattgat

SEQ ID NO: 552 - RNA, 21 nt, synthetic construct
  misc_feature 1..21: note = artificial sequence
  source 1..21: mol_type = other RNA
gaggctacggaaactctgatt

SEQ ID NO: 553 - RNA, 21 nt, synthetic construct
  misc_feature 1..21: note = artificial sequence
  source 1..21: mol_type = other RNA
tgcaaaacacaatgttgataa

SEQ ID NO: 554 - RNA, 21 nt, synthetic construct
  misc_feature 1..21: note = artificial sequence
  source 1..21: mol_type = other RNA
ttcgttgaatgacgacttgaa

SEQ ID NO: 555 - RNA, 21 nt, synthetic construct
  misc_feature 1..21: note = artificial sequence
  source 1..21: mol_type = other RNA
atggtagaaacaactgcctat

SEQ ID NO: 556 - RNA, 21 nt, synthetic construct
  misc_feature 1..21: note = artificial sequence
  source 1..21: mol_type = other RNA
tagaaacaactgcctatgctt

SEQ ID NO: 557 - RNA, 21 nt, synthetic construct
  misc_feature 1..21: note = artificial sequence
  source 1..21: mol_type = other RNA
acagcttttgctttaagagta

SEQ ID NO: 558 - RNA, 21 nt, synthetic construct
  misc_feature 1..21: note = artificial sequence
  source 1..21: mol_type = other RNA
gatgcatcttatcaaagtata

SEQ ID NO: 559 - RNA, 21 nt, synthetic construct
  misc_feature 1..21: note = artificial sequence
  source 1..21: mol_type = other RNA
tatggacatcgatgtttctta

SEQ ID NO: 560 - RNA, 21 nt, synthetic construct
  misc_feature 1..21: note = artificial sequence
  source 1..21: mol_type = other RNA
atagcacttccaatatcaaaa

SEQ ID NO: 561 - RNA, 21 nt, synthetic construct
  misc_feature 1..21: note = artificial sequence
  source 1..21: mol_type = other RNA
cacccatttatcgtttttgga

SEQ ID NO: 562 - RNA, 21 nt, synthetic construct
  misc_feature 1..21: note = artificial sequence
  source 1..21: mol_type = other RNA
cagaaccaaaattcaatttgt

SEQ ID NO: 563 - RNA, 21 nt, synthetic construct
  misc_feature 1..21: note = artificial sequence
  source 1..21: mol_type = other RNA
actggagacttggtttggaaa

SEQ ID NO: 564 - RNA, 21 nt, synthetic construct
  misc_feature 1..21: note = artificial sequence
  source 1..21: mol_type = other RNA
acatgttattctgcaactgaa

SEQ ID NO: 565 - RNA, 21 nt, synthetic construct
  misc_feature 1..21: note = artificial sequence
  source 1..21: mol_type = other RNA
aaagctcactttacttagaat

SEQ ID NO: 566 - RNA, 21 nt, synthetic construct
  misc_feature 1..21: note = artificial sequence
  source 1..21: mol_type = other RNA
cagcttatatcttacagcctt

SEQ ID NO: 567 - RNA, 21 nt, synthetic construct
  misc_feature 1..21: note = artificial sequence
  source 1..21: mol_type = other RNA
cccattaattgaaaagcagaa

SEQ ID NO: 568 - RNA, 21 nt, synthetic construct
  misc_feature 1..21: note = artificial sequence
  source 1..21: mol_type = other RNA
ctgtagaaaatgtttttgtca

SEQ ID NO: 569 - RNA, 21 nt, synthetic construct
  misc_feature 1..21: note = artificial sequence
  source 1..21: mol_type = other RNA
atctctgcagagacaagaaaa

SEQ ID NO: 570 - RNA, 21 nt, synthetic construct
  misc_feature 1..21: note = artificial sequence
  source 1..21: mol_type = other RNA
caatcttaacaatacaaccaa

SEQ ID NO: 571 - RNA, 21 nt, synthetic construct
  misc_feature 1..21: note = artificial sequence
  source 1..21: mol_type = other RNA
taattataaaatgacagacaa

SEQ ID NO: 572 - RNA, 21 nt, synthetic construct
  misc_feature 1..21: note = artificial sequence
  source 1..21: mol_type = other RNA
ttctgacccattaattgaaaa

SEQ ID NO: 573 - RNA, 21 nt, synthetic construct
  misc_feature 1..21: note = artificial sequence
  source 1..21: mol_type = other RNA
atgttcatcgtgtcaagcatt

SEQ ID NO: 574 - RNA, 21 nt, synthetic construct
  misc_feature 1..21: note = artificial sequence
  source 1..21: mol_type = other RNA
atcttaacaatacaaccaaaa

SEQ ID NO: 575 - RNA, 21 nt, synthetic construct
  misc_feature 1..21: note = artificial sequence
  source 1..21: mol_type = other RNA
aaccatgggccttttgggaat

SEQ ID NO: 576 - RNA, 21 nt, synthetic construct
  misc_feature 1..21: note = artificial sequence
  source 1..21: mol_type = other RNA
ttgcacttatggactcctgtt

SEQ ID NO: 577 - RNA, 21 nt, synthetic construct
  misc_feature 1..21: note = artificial sequence
  source 1..21: mol_type = other RNA
cagaggctacggaaactctga

SEQ ID NO: 578 - RNA, 21 nt, synthetic construct
  misc_feature 1..21: note = artificial sequence
  source 1..21: mol_type = other RNA
ttggatctgacaatctctgca

SEQ ID NO: 579 - RNA, 21 nt, synthetic construct
  misc_feature 1..21: note = artificial sequence
  source 1..21: mol_type = other RNA
aaaagcagaaactgaagaaaa

SEQ ID NO: 580 - RNA, 21 nt, synthetic construct
  misc_feature 1..21: note = artificial sequence
  source 1..21: mol_type = other RNA
aagttgacatggtagaagaaa

SEQ ID NO: 581 - RNA, 21 nt, synthetic construct
  misc_feature 1..21: note = artificial sequence
  source 1..21: mol_type = other RNA
aacatctgaatattattgtta

SEQ ID NO: 582 - RNA, 21 nt, synthetic construct
  misc_feature 1..21: note = artificial sequence
  source 1..21: mol_type = other RNA
acaggaaatcattggaacatt

SEQ ID NO: 583 - RNA, 21 nt, synthetic construct
  misc_feature 1..21: note = artificial sequence
  source 1..21: mol_type = other RNA
gtcatcaaatggctatcagaa

SEQ ID NO: 584 - RNA, 21 nt, synthetic construct
  misc_feature 1..21: note = artificial sequence
  source 1..21: mol_type = other RNA
caagggcaaaattatccactt

SEQ ID NO: 585 - RNA, 21 nt, synthetic construct
  misc_feature 1..21: note = artificial sequence
  source 1..21: mol_type = other RNA
ccagtaacactgaatgcacaa

SEQ ID NO: 586 - RNA, 21 nt, synthetic construct
  misc_feature 1..21: note = artificial sequence
  source 1..21: mol_type = other RNA
gccgcgtgcaagtgtgtagaa

SEQ ID NO: 587 - RNA, 21 nt, synthetic construct
  misc_feature 1..21: note = artificial sequence
  source 1..21: mol_type = other RNA
tgggccttttgggaatacttt

SEQ ID NO: 588 - RNA, 21 nt, synthetic construct
  misc_feature 1..21: note = artificial sequence
  source 1..21: mol_type = other RNA
aggctacggaaactctgatta

SEQ ID NO: 589 - RNA, 21 nt, synthetic construct
  misc_feature 1..21: note = artificial sequence
  source 1..21: mol_type = other RNA
taacactgaatgcacaaacaa

SEQ ID NO: 590 - RNA, 21 nt, synthetic construct
  misc_feature 1..21: note = artificial sequence
  source 1..21: mol_type = other RNA
aacccagtttcttatgtgtat

SEQ ID NO: 591 - RNA, 21 nt, synthetic construct
  misc_feature 1..21: note = artificial sequence
  source 1..21: mol_type = other RNA
gtgtctgattcagtctggtta

SEQ ID NO: 592 - RNA, 21 nt, synthetic construct
  misc_feature 1..21: note = artificial sequence
  source 1..21: mol_type = other RNA
aaaggaactgtttacaactat

SEQ ID NO: 593 - RNA, 21 nt, synthetic construct
  misc_feature 1..21: note = artificial sequence
  source 1..21: mol_type = other RNA
taaggctttgctagtgggaga

SEQ ID NO: 594 - RNA, 21 nt, synthetic construct
  misc_feature 1..21: note = artificial sequence
  source 1..21: mol_type = other RNA
cggatatttgaactctttgaa

SEQ ID NO: 595 - RNA, 21 nt, synthetic construct
  misc_feature 1..21: note = artificial sequence
  source 1..21: mol_type = other RNA
gagcagcgagctgcacggatt

SEQ ID NO: 596 - RNA, 21 nt, synthetic construct
  misc_feature 1..21: note = artificial sequence
  source 1..21: mol_type = other RNA
ctacagtttagaagatttaaa

SEQ ID NO: 597 - RNA, 21 nt, synthetic construct
  misc_feature 1..21: note = artificial sequence
  source 1..21: mol_type = other RNA
atgtaaaccagagattgcata

SEQ ID NO: 598 - RNA, 21 nt, synthetic construct
  misc_feature 1..21: note = artificial sequence
  source 1..21: mol_type = other RNA
gagcaaacatatgtcatttca

SEQ ID NO: 599 - RNA, 21 nt, synthetic construct
  misc_feature 1..21: note = artificial sequence
  source 1..21: mol_type = other RNA
ttgctgagaaagactctgaga

SEQ ID NO: 600 - RNA, 21 nt, synthetic construct
  misc_feature 1..21: note = artificial sequence
  source 1..21: mol_type = other RNA
gtgttggagcatctgaaaata

SEQ ID NO: 601 - RNA, 21 nt, synthetic construct
  misc_feature 1..21: note = artificial sequence
  source 1..21: mol_type = other RNA
ctctattaaaagttatcctga

SEQ ID NO: 602 - RNA, 21 nt, synthetic construct
  misc_feature 1..21: note = artificial sequence
  source 1..21: mol_type = other RNA
tggaaagacaatcttcagcat

SEQ ID NO: 603 - RNA, 21 nt, synthetic construct
  misc_feature 1..21: note = artificial sequence
  source 1..21: mol_type = other RNA
ccgtgttggagcatctgaaaa

SEQ ID NO: 604 - RNA, 21 nt, synthetic construct
  misc_feature 1..21: note = artificial sequence
  source 1..21: mol_type = other RNA
agggaaagctattctggtgtt

SEQ ID NO: 605 - RNA, 21 nt, synthetic construct
  misc_feature 1..21: note = artificial sequence
  source 1..21: mol_type = other RNA
aacagcaatgcaaaacacaat

SEQ ID NO: 606 - RNA, 21 nt, synthetic construct
  misc_feature 1..21: note = artificial sequence
  source 1..21: mol_type = other RNA
ttgctcaagtcacatttgatt

SEQ ID NO: 607 - RNA, 21 nt, synthetic construct
  misc_feature 1..21: note = artificial sequence
  source 1..21: mol_type = other RNA
tctgaggaagtttgcagcttt

SEQ ID NO: 608 - RNA, 21 nt, synthetic construct
  misc_feature 1..21: note = artificial sequence
  source 1..21: mol_type = other RNA
atggaattgctcaagtcacat

SEQ ID NO: 609 - RNA, 21 nt, synthetic construct
  misc_feature 1..21: note = artificial sequence
  source 1..21: mol_type = other RNA
acgtttatatcacatttggaa

SEQ ID NO: 610 - RNA, 21 nt, synthetic construct
  misc_feature 1..21: note = artificial sequence
  source 1..21: mol_type = other RNA
caagccagaaattcggagtta

SEQ ID NO: 611 - RNA, 21 nt, synthetic construct
  misc_feature 1..21: note = artificial sequence
  source 1..21: mol_type = other RNA
gtccccaaaacagaaatcaaa

SEQ ID NO: 612 - RNA, 21 nt, synthetic construct
  misc_feature 1..21: note = artificial sequence
  source 1..21: mol_type = other RNA
atgcaaaacacaatgttgata

SEQ ID NO: 613 - RNA, 21 nt, synthetic construct
  misc_feature 1..21: note = artificial sequence
  source 1..21: mol_type = other RNA
caaaacagaaatcaaaaggat

SEQ ID NO: 614 - RNA, 21 nt, synthetic construct
  misc_feature 1..21: note = artificial sequence
  source 1..21: mol_type = other RNA
aggctgacgtttatatcacat

SEQ ID NO: 615 - RNA, 21 nt, synthetic construct
  misc_feature 1..21: note = artificial sequence
  source 1..21: mol_type = other RNA
tacgatggagcctgcgttaat

SEQ ID NO: 616 - RNA, 21 nt, synthetic construct
  misc_feature 1..21: note = artificial sequence
  source 1..21: mol_type = other RNA
aagccctccagataaaataca

SEQ ID NO: 617 - RNA, 21 nt, synthetic construct
  misc_feature 1..21: note = artificial sequence
  source 1..21: mol_type = other RNA
taacaactgtagttcacaaaa

SEQ ID NO: 618 - RNA, 21 nt, synthetic construct
  misc_feature 1..21: note = artificial sequence
  source 1..21: mol_type = other RNA
ttgaaagatataaattatgtt

SEQ ID NO: 619 - RNA, 21 nt, synthetic construct
  misc_feature 1..21: note = artificial sequence
  source 1..21: mol_type = other RNA
ttgcagcttttatttgaaaat

SEQ ID NO: 620 - RNA, 21 nt, synthetic construct
  misc_feature 1..21: note = artificial sequence
  source 1..21: mol_type = other RNA
tctgaaaatattgtgattcaa

SEQ ID NO: 621 - RNA, 21 nt, synthetic construct
  misc_feature 1..21: note = artificial sequence
  source 1..21: mol_type = other RNA
cagaagaaaacagttgcagtt

SEQ ID NO: 622 - RNA, 21 nt, synthetic construct
  misc_feature 1..21: note = artificial sequence
  source 1..21: mol_type = other RNA
aacagcatgtaaaccagagat

SEQ ID NO: 623 - RNA, 21 nt, synthetic construct
  misc_feature 1..21: note = artificial sequence
  source 1..21: mol_type = other RNA
atcacatccatcactgtagaa

SEQ ID NO: 624 - RNA, 21 nt, synthetic construct
  misc_feature 1..21: note = artificial sequence
  source 1..21: mol_type = other RNA
caagaaaacaaacagcatgta

SEQ ID NO: 625 - RNA, 21 nt, synthetic construct
  misc_feature 1..21: note = artificial sequence
  source 1..21: mol_type = other RNA
taggtgagatcttgtctgcag

SEQ ID NO: 626 - RNA, 21 nt, synthetic construct
  misc_feature 1..21: note = artificial sequence
  source 1..21: mol_type = other RNA
ctcctccaaggttattggaca

SEQ ID NO: 627 - RNA, 21 nt, synthetic construct
  misc_feature 1..21: note = artificial sequence
  source 1..21: mol_type = other RNA
attggacaccggaaacaataa

SEQ ID NO: 628 - RNA, 21 nt, synthetic construct
  misc_feature 1..21: note = artificial sequence
  source 1..21: mol_type = other RNA
ctgaatgttgtgtcgtcgcaa

SEQ ID NO: 629 - RNA, 21 nt, synthetic construct
  misc_feature 1..21: note = artificial sequence
  source 1..21: mol_type = other RNA
agcataaaggtgccttacata

SEQ ID NO: 630 - RNA, 21 nt, synthetic construct
  misc_feature 1..21: note = artificial sequence
  source 1..21: mol_type = other RNA
aagaacgctgcaaaagaagat

SEQ ID NO: 631 - RNA, 21 nt, synthetic construct
  misc_feature 1..21: note = artificial sequence
  source 1..21: mol_type = other RNA
aactttaagaattttgaaatt

SEQ ID NO: 632 - RNA, 21 nt, synthetic construct
  misc_feature 1..21: note = artificial sequence
  source 1..21: mol_type = other RNA
atgctttactcaccagtctga

SEQ ID NO: 633 - RNA, 21 nt, synthetic construct
  misc_feature 1..21: note = artificial sequence
  source 1..21: mol_type = other RNA
ttccttgacctggattgaata

SEQ ID NO: 634 - RNA, 21 nt, synthetic construct
  misc_feature 1..21: note = artificial sequence
  source 1..21: mol_type = other RNA
tggaagttgtatcaaagcatt

SEQ ID NO: 635 - RNA, 21 nt, synthetic construct
  misc_feature 1..21: note = artificial sequence
  source 1..21: mol_type = other RNA
attgggaaggctacacatgaa

SEQ ID NO: 636 - RNA, 21 nt, synthetic construct
  misc_feature 1..21: note = artificial sequence
  source 1..21: mol_type = other RNA
gccaaactgtgtctcttaata

SEQ ID NO: 637 - RNA, 21 nt, synthetic construct
  misc_feature 1..21: note = artificial sequence
  source 1..21: mol_type = other RNA
cttgcttttattagagaatga

SEQ ID NO: 638 - RNA, 21 nt, synthetic construct
  misc_feature 1..21: note = artificial sequence
  source 1..21: mol_type = other RNA
cctggattgaatactggccta

SEQ ID NO: 639 - RNA, 21 nt, synthetic construct
  misc_feature 1..21: note = artificial sequence
  source 1..21: mol_type = other RNA
ctccgtgctaatatctctcat

SEQ ID NO: 640 - RNA, 21 nt, synthetic construct
  misc_feature 1..21: note = artificial sequence
  source 1..21: mol_type = other RNA
ttggccttcacaacatcaatt

SEQ ID NO: 641 - RNA, 21 nt, synthetic construct
  misc_feature 1..21: note = artificial sequence
  source 1..21: mol_type = other RNA
ttcaacaactggaaccgcata

SEQ ID NO: 642 - RNA, 21 nt, synthetic construct
  misc_feature 1..21: note = artificial sequence
  source 1..21: mol_type = other RNA
ctggagtttaatgtcaaaact

SEQ ID NO: 643 - RNA, 21 nt, synthetic construct
  misc_feature 1..21: note = artificial sequence
  source 1..21: mol_type = other RNA
atgtcatttcagcaccaaaaa

SEQ ID NO: 644 - RNA, 21 nt, synthetic construct
  misc_feature 1..21: note = artificial sequence
  source 1..21: mol_type = other RNA
acagtccattgaaagggagta

SEQ ID NO: 645 - RNA, 21 nt, synthetic construct
  misc_feature 1..21: note = artificial sequence
  source 1..21: mol_type = other RNA
tagggccaagatgcatcaaag

SEQ ID NO: 646 - RNA, 21 nt, synthetic construct
  misc_feature 1..21: note = artificial sequence
  source 1..21: mol_type = other RNA
ttcacagtatcaaccaataaa

SEQ ID NO: 647 - RNA, 21 nt, synthetic construct
  misc_feature 1..21: note = artificial sequence
  source 1..21: mol_type = other RNA
aaaattcacagtatcaaccaa

SEQ ID NO: 648 - RNA, 21 nt, synthetic construct
  misc_feature 1..21: note = artificial sequence
  source 1..21: mol_type = other RNA
tggaataagagaagacttaaa

SEQ ID NO: 649 - RNA, 21 nt, synthetic construct
  misc_feature 1..21: note = artificial sequence
  source 1..21: mol_type = other RNA
atgttttatagcacttccaat

SEQ ID NO: 650 - RNA, 21 nt, synthetic construct
  misc_feature 1..21: note = artificial sequence
  source 1..21: mol_type = other RNA
caaaatcaaaaagaatgccaa

SEQ ID NO: 651 - RNA, 21 nt, synthetic construct
  misc_feature 1..21: note = artificial sequence
  source 1..21: mol_type = other RNA
aaggggcctcttcttaacaaa

SEQ ID NO: 652 - RNA, 21 nt, synthetic construct
  misc_feature 1..21: note = artificial sequence
  source 1..21: mol_type = other RNA
aaggaatcagaatgaacaaaa

SEQ ID NO: 653 - RNA, 21 nt, synthetic construct
  misc_feature 1..21: note = artificial sequence
  source 1..21: mol_type = other RNA
ctccagataaagcttcagtta

SEQ ID NO: 654 - RNA, 21 nt, synthetic construct
  misc_feature 1..21: note = artificial sequence
  source 1..21: mol_type = other RNA
caagggcgtgttcgtgctgaa

SEQ ID NO: 655 - RNA, 21 nt, synthetic construct
  misc_feature 1..21: note = artificial sequence
  source 1..21: mol_type = other RNA
gaggagaattgcttcatacaa

SEQ ID NO: 656 - RNA, 21 nt, synthetic construct
  misc_feature 1..21: note = artificial sequence
  source 1..21: mol_type = other RNA
tacgaagctcatgaatatatt

SEQ ID NO: 657 - RNA, 21 nt, synthetic construct
  misc_feature 1..21: note = artificial sequence
  source 1..21: mol_type = other RNA
cacggagaagcgaatggacaa

SEQ ID NO: 658 - RNA, 21 nt, synthetic construct
  misc_feature 1..21: note = artificial sequence
  source 1..21: mol_type = other RNA
ttggctcaatgaacagagata

SEQ ID NO: 659 - RNA, 21 nt, synthetic construct
  misc_feature 1..21: note = artificial sequence
  source 1..21: mol_type = other RNA
taggaacaccctcatcatcta

SEQ ID NO: 660 - RNA, 21 nt, synthetic construct
  misc_feature 1..21: note = artificial sequence
  source 1..21: mol_type = other RNA
ctggccaatggtgttgacaga

SEQ ID NO: 661 - RNA, 21 nt, synthetic construct
  misc_feature 1..21: note = artificial sequence
  source 1..21: mol_type = other RNA
cacggtcatcgctgtgcatta

SEQ ID NO: 662 - RNA, 21 nt, synthetic construct
  misc_feature 1..21: note = artificial sequence
  source 1..21: mol_type = other RNA
cagggagttcaagtcagaaaa

SEQ ID NO: 663 - RNA, 21 nt, synthetic construct
  misc_feature 1..21: note = artificial sequence
  source 1..21: mol_type = other RNA
ccggacggtcatggtcaacat

SEQ ID NO: 664 - RNA, 21 nt, synthetic construct
  misc_feature 1..21: note = artificial sequence
  source 1..21: mol_type = other RNA
ccagataaagcttcagttata

SEQ ID NO: 665 - RNA, 21 nt, synthetic construct
  misc_feature 1..21: note = artificial sequence
  source 1..21: mol_type = other RNA
caggaggatgcgcccgtgata

SEQ ID NO: 666 - RNA, 21 nt, synthetic construct
  misc_feature 1..21: note = artificial sequence
  source 1..21: mol_type = other RNA
ttggtggattacggaacaaca

SEQ ID NO: 667 - RNA, 21 nt, synthetic construct
  misc_feature 1..21: note = artificial sequence
  source 1..21: mol_type = other RNA
cagcaccgtgggcaactccaa

SEQ ID NO: 668 - RNA, 21 nt, synthetic construct
  misc_feature 1..21: note = artificial sequence
  source 1..21: mol_type = other RNA
aggagaattgcttcatacaaa

SEQ ID NO: 669 - RNA, 21 nt, synthetic construct
  misc_feature 1..21: note = artificial sequence
  source 1..21: mol_type = other RNA
tccggaaaaggaggatggaaa

SEQ ID NO: 670 - RNA, 21 nt, synthetic construct
  misc_feature 1..21: note = artificial sequence
  source 1..21: mol_type = other RNA
cacctacctgatcatgaacaa

SEQ ID NO: 671 - RNA, 21 nt, synthetic construct
  misc_feature 1..21: note = artificial sequence
  source 1..21: mol_type = other RNA
ctccactgagtttgaggtgaa

SEQ ID NO: 672 - RNA, 21 nt, synthetic construct
  misc_feature 1..21: note = artificial sequence
  source 1..21: mol_type = other RNA
ctcatgaatatatttttgaaa

SEQ ID NO: 673 - RNA, 21 nt, synthetic construct
  misc_feature 1..21: note = artificial sequence
  source 1..21: mol_type = other RNA
caagaaatgattggtggatta

SEQ ID NO: 674 - RNA, 21 nt, synthetic construct
  misc_feature 1..21: note = artificial sequence
  source 1..21: mol_type = other RNA
ctcgggctgcggggaacagaa

SEQ ID NO: 675 - RNA, 21 nt, synthetic construct
  misc_feature 1..21: note = artificial sequence
  source 1..21: mol_type = other RNA
caggagtaacctggatgagga

SEQ ID NO: 676 - RNA, 21 nt, synthetic construct
  misc_feature 1..21: note = artificial sequence
  source 1..21: mol_type = other RNA
gaggacgaatgccaagacgaa

SEQ ID NO: 677 - RNA, 21 nt, synthetic construct
  misc_feature 1..21: note = artificial sequence
  source 1..21: mol_type = other RNA
caagacgaagagaaccagaaa

SEQ ID NO: 678 - RNA, 21 nt, synthetic construct
  misc_feature 1..21: note = artificial sequence
  source 1..21: mol_type = other RNA
caccgagagcatggttgtctt

SEQ ID NO: 679 - RNA, 21 nt, synthetic construct
  misc_feature 1..21: note = artificial sequence
  source 1..21: mol_type = other RNA
ctgcagctcagctccatccta

SEQ ID NO: 680 - RNA, 21 nt, synthetic construct
  misc_feature 1..21: note = artificial sequence
  source 1..21: mol_type = other RNA
gtggaacgttgaggacttgaa

SEQ ID NO: 681 - RNA, 21 nt, synthetic construct
  misc_feature 1..21: note = artificial sequence
  source 1..21: mol_type = other RNA
caggatgccactatgtctata

SEQ ID NO: 682 - RNA, 21 nt, synthetic construct
  misc_feature 1..21: note = artificial sequence
  source 1..21: mol_type = other RNA
ctgtaataaattcgacctcaa

SEQ ID NO: 683 - RNA, 21 nt, synthetic construct
  misc_feature 1..21: note = artificial sequence
  source 1..21: mol_type = other RNA
cacgacttcccaggcaaaaaa

SEQ ID NO: 684 - RNA, 21 nt, synthetic construct
  misc_feature 1..21: note = artificial sequence
  source 1..21: mol_type = other RNA
tacctgcatctctcagtgcta

SEQ ID NO: 685 - RNA, 21 nt, synthetic construct
  misc_feature 1..21: note = artificial sequence
  source 1..21: mol_type = other RNA
cagctacatcatcgggaagga

SEQ ID NO: 686 - RNA, 21 nt, synthetic construct
  misc_feature 1..21: note = artificial sequence
  source 1..21: mol_type = other RNA
gtccaatgactttgacgagta

SEQ ID NO: 687 - RNA, 21 nt, synthetic construct
  misc_feature 1..21: note = artificial sequence
  source 1..21: mol_type = other RNA
accctactctgttgttcgaaa

SEQ ID NO: 688 - RNA, 21 nt, synthetic construct
  misc_feature 1..21: note = artificial sequence
  source 1..21: mol_type = other RNA
ccgagccgttctctacaatta

SEQ ID NO: 689 - RNA, 21 nt, synthetic construct
  misc_feature 1..21: note = artificial sequence
  source 1..21: mol_type = other RNA
acggagaagcgaatggacaaa

SEQ ID NO: 690 - RNA, 21 nt, synthetic construct
  misc_feature 1..21: note = artificial sequence
  source 1..21: mol_type = other RNA
cacagtcacagctgaaggaaa

SEQ ID NO: 691 - RNA, 21 nt, synthetic construct
  misc_feature 1..21: note = artificial sequence
  source 1..21: mol_type = other RNA
agggcgtgttcgtgctgaata

SEQ ID NO: 692 - RNA, 21 nt, synthetic construct
  misc_feature 1..21: note = artificial sequence
  source 1..21: mol_type = other RNA
gggcgtgttcgtgctgaataa

SEQ ID NO: 693 - RNA, 21 nt, synthetic construct
  misc_feature 1..21: note = artificial sequence
  source 1..21: mol_type = other RNA
aagatcaactcacctgtaata

SEQ ID NO: 694 - RNA, 21 nt, synthetic construct
  misc_feature 1..21: note = artificial sequence
  source 1..21: mol_type = other RNA
aaggaaaatgagggtttcaca

SEQ ID NO: 695 - RNA, 21 nt, synthetic construct
  misc_feature 1..21: note = artificial sequence
  source 1..21: mol_type = other RNA
aagccttggctcaataccaaa

SEQ ID NO: 696 - RNA, 21 nt, synthetic construct
  misc_feature 1..21: note = artificial sequence
  source 1..21: mol_type = other RNA
caagccttggctcaataccaa

SEQ ID NO: 697 - RNA, 21 nt, synthetic construct
  misc_feature 1..21: note = artificial sequence
  source 1..21: mol_type = other RNA
cccgggcagtgggaaggatta

SEQ ID NO: 698 - RNA, 21 nt, synthetic construct
  misc_feature 1..21: note = artificial sequence
  source 1..21: mol_type = other RNA
ccggcagaaccaagagctcaa

SEQ ID NO: 699 - RNA, 21 nt, synthetic construct
  misc_feature 1..21: note = artificial sequence
  source 1..21: mol_type = other RNA
cagggctgaccttcacgagca

SEQ ID NO: 700 - RNA, 21 nt, synthetic construct
  misc_feature 1..21: note = artificial sequence
  source 1..21: mol_type = other RNA
aaccagcaccggaaacagaaa

SEQ ID NO: 701 - RNA, 21 nt, synthetic construct
  misc_feature 1..21: note = artificial sequence
  source 1..21: mol_type = other RNA
cagtgggagaagttcggccta

SEQ ID NO: 702 - RNA, 21 nt, synthetic construct
  misc_feature 1..21: note = artificial sequence
  source 1..21: mol_type = other RNA
caggtggaaatccgagccgtt

SEQ ID NO: 703 - RNA, 21 nt, synthetic construct
  misc_feature 1..21: note = artificial sequence
  source 1..21: mol_type = other RNA
caccgaaaaatggaatctcta

SEQ ID NO: 704 - RNA, 21 nt, synthetic construct
  misc_feature 1..21: note = artificial sequence
  source 1..21: mol_type = other RNA
ctctacgaagctcatgaatat

SEQ ID NO: 705 - RNA, 21 nt, synthetic construct
  misc_feature 1..21: note = artificial sequence
  source 1..21: mol_type = other RNA
ctggctgtggaacgttgagga

SEQ ID NO: 706 - RNA, 21 nt, synthetic construct
  misc_feature 1..21: note = artificial sequence
  source 1..21: mol_type = other RNA
cagctaaaagactttgacttt

SEQ ID NO: 707 - RNA, 21 nt, synthetic construct
  misc_feature 1..21: note = artificial sequence
  source 1..21: mol_type = other RNA
acggtggtggctatggctcta

SEQ ID NO: 708 - RNA, 21 nt, synthetic construct
  misc_feature 1..21: note = artificial sequence
  source 1..21: mol_type = other RNA
ctccgtgtgggtggacgtcaa

SEQ ID NO: 709 - RNA, 21 nt, synthetic construct
  misc_feature 1..21: note = artificial sequence
  source 1..21: mol_type = other RNA
agcagtcaaggtctacgccta

SEQ ID NO: 710 - RNA, 21 nt, synthetic construct
  misc_feature 1..21: note = artificial sequence
  source 1..21: mol_type = other RNA
agctttcaaagttcaccaata

SEQ ID NO: 711 - RNA, 21 nt, synthetic construct
  misc_feature 1..21: note = artificial sequence
  source 1..21: mol_type = other RNA
tagctttcaaagttcaccaat

SEQ ID NO: 712 - RNA, 21 nt, synthetic construct
  misc_feature 1..21: note = artificial sequence
  source 1..21: mol_type = other RNA
cactctgaggatgactgtcta

SEQ ID NO: 713 - RNA, 21 nt, synthetic construct
  misc_feature 1..21: note = artificial sequence
  source 1..21: mol_type = other RNA
caccctcatcatctacctgga

SEQ ID NO: 714 - RNA, 21 nt, synthetic construct
  misc_feature 1..21: note = artificial sequence
  source 1..21: mol_type = other RNA
aagagccaccgaaaaatggaa

SEQ ID NO: 715 - RNA, 21 nt, synthetic construct
  misc_feature 1..21: note = artificial sequence
  source 1..21: mol_type = other RNA
gccggaaggaatcagaatgaa

SEQ ID NO: 716 - RNA, 21 nt, synthetic construct
  misc_feature 1..21: note = artificial sequence
  source 1..21: mol_type = other RNA
caccttcacgatcccagccaa

SEQ ID NO: 717 - RNA, 21 nt, synthetic construct
  misc_feature 1..21: note = artificial sequence
  source 1..21: mol_type = other RNA
ctggaggcccacgacgcgcaa

SEQ ID NO: 718 - RNA, 21 nt, synthetic construct
  misc_feature 1..21: note = artificial sequence
  source 1..21: mol_type = other RNA
atggaatctctacgaagctca

SEQ ID NO: 719 - RNA, 21 nt, synthetic construct
  misc_feature 1..21: note = artificial sequence
  source 1..21: mol_type = other RNA
cacgttcatcagccccatcaa

SEQ ID NO: 720 - RNA, 21 nt, synthetic construct
  misc_feature 1..21: note = artificial sequence
  source 1..21: mol_type = other RNA
tcgctgcaggaggctaaagat

SEQ ID NO: 721 - RNA, 21 nt, synthetic construct
  misc_feature 1..21: note = artificial sequence
  source 1..21: mol_type = other RNA
caaggtctacgcctattacaa

SEQ ID NO: 722 - RNA, 21 nt, synthetic construct
  misc_feature 1..21: note = artificial sequence
  source 1..21: mol_type = other RNA
ctcagctccatcctacttgaa

SEQ ID NO: 723 - RNA, 21 nt, synthetic construct
  misc_feature 1..21: note = artificial sequence
  source 1..21: mol_type = other RNA
gagggtggaactactccacaa

SEQ ID NO: 724 - RNA, 21 nt, synthetic construct
  misc_feature 1..21: note = artificial sequence
  source 1..21: mol_type = other RNA
cagggagatggcgtggccaaa

SEQ ID NO: 725 - RNA, 21 nt, synthetic construct
  misc_feature 1..21: note = artificial sequence
  source 1..21: mol_type = other RNA
cggggagaccctcaacgtcaa

SEQ ID NO: 726 - RNA, 21 nt, synthetic construct
  misc_feature 1..21: note = artificial sequence
  source 1..21: mol_type = other RNA
caggagtggactatgtgtaca

SEQ ID NO: 727 - RNA, 21 nt, synthetic construct
  misc_feature 1..21: note = artificial sequence
  source 1..21: mol_type = other RNA
ctgaggatgactgtctagctt

SEQ ID NO: 728 - RNA, 21 nt, synthetic construct
  misc_feature 1..21: note = artificial sequence
  source 1..21: mol_type = other RNA
ctgcaggaggctaaagatatt

SEQ ID NO: 729 - RNA, 21 nt, synthetic construct
  misc_feature 1..21: note = artificial sequence
  source 1..21: mol_type = other RNA
caccgtgggcaactccaacaa

SEQ ID NO: 730 - RNA, 21 nt, synthetic construct
  misc_feature 1..21: note = artificial sequence
  source 1..21: mol_type = other RNA
ctgaagctggaggagaagaaa

SEQ ID NO: 731 - RNA, 21 nt, synthetic construct
  misc_feature 1..21: note = artificial sequence
  source 1..21: mol_type = other RNA
ctcaggatgccaagaacacta

SEQ ID NO: 732 - RNA, 21 nt, synthetic construct
  misc_feature 1..21: note = artificial sequence
  source 1..21: mol_type = other RNA
acgggaagaaagtggagggaa

SEQ ID NO: 733 - RNA, 21 nt, synthetic construct
  misc_feature 1..21: note = artificial sequence
  source 1..21: mol_type = other RNA
ctagctttcaaagttcaccaa

SEQ ID NO: 734 - RNA, 21 nt, synthetic construct
  misc_feature 1..21: note = artificial sequence
  source 1..21: mol_type = other RNA
aaggtctcacactctgaggat

SEQ ID NO: 735 - RNA, 21 nt, synthetic construct
  misc_feature 1..21: note = artificial sequence
  source 1..21: mol_type = other RNA
gtgccgctgtgctgaggagaa

SEQ ID NO: 736 - RNA, 21 nt, synthetic construct
  misc_feature 1..21: note = artificial sequence
  source 1..21: mol_type = other RNA
cgggacccaagtggtggagaa

SEQ ID NO: 737 - RNA, 21 nt, synthetic construct
  misc_feature 1..21: note = artificial sequence
  source 1..21: mol_type = other RNA
atggctgatcctggagaagca

SEQ ID NO: 738 - RNA, 21 nt, synthetic construct
  misc_feature 1..21: note = artificial sequence
  source 1..21: mol_type = other RNA
ctggatgaggacatcattgca

SEQ ID NO: 739 - RNA, 21 nt, synthetic construct
  misc_feature 1..21: note = artificial sequence
  source 1..21: mol_type = other RNA
aagggcgtgttcgtgctgaat

SEQ ID NO: 740 - RNA, 21 nt, synthetic construct
  misc_feature 1..21: note = artificial sequence
  source 1..21: mol_type = other RNA
tacagagaaattctactacat

SEQ ID NO: 741 - RNA, 21 nt, synthetic construct
  misc_feature 1..21: note = artificial sequence
  source 1..21: mol_type = other RNA
gacgaagagaaccagaaacaa

SEQ ID NO: 742 - RNA, 21 nt, synthetic construct
  misc_feature 1..21: note = artificial sequence
  source 1..21: mol_type = other RNA
tccatcctacttgaaacctca

SEQ ID NO: 743 - RNA, 21 nt, synthetic construct
  misc_feature 1..21: note = artificial sequence
  source 1..21: mol_type = other RNA
acggaacaacaacgagaaaga

SEQ ID NO: 744 - RNA, 21 nt, synthetic construct
  misc_feature 1..21: note = artificial sequence
  source 1..21: mol_type = other RNA
aatgattggtggattacggaa

SEQ ID NO: 745 - RNA, 21 nt, synthetic construct
  misc_feature 1..21: note = artificial sequence
  source 1..21: mol_type = other RNA
aacgagcaggtggaaatccga

SEQ ID NO: 746 - RNA, 21 nt, synthetic construct
  misc_feature 1..21: note = artificial sequence
  source 1..21: mol_type = other RNA
agccctgaagctggaggagaa

SEQ ID NO: 747 - RNA, 21 nt, synthetic construct
  misc_feature 1..21: note = artificial sequence
  source 1..21: mol_type = other RNA
gtcctcgttgtccgttccata

SEQ ID NO: 748 - RNA, 21 nt, synthetic construct
  misc_feature 1..21: note = artificial sequence
  source 1..21: mol_type = other RNA
cagcagctggccttcagacaa

SEQ ID NO: 749 - RNA, 21 nt, synthetic construct
  misc_feature 1..21: note = artificial sequence
  source 1..21: mol_type = other RNA
ctccaagtatgagctggacaa

SEQ ID NO: 750 - RNA, 21 nt, synthetic construct
  misc_feature 1..21: note = artificial sequence
  source 1..21: mol_type = other RNA
ttcaaagttcaccaatacttt

SEQ ID NO: 751 - RNA, 21 nt, synthetic construct
  misc_feature 1..21: note = artificial sequence
  source 1..21: mol_type = other RNA
accataaaaccagcaccggaa

SEQ ID NO: 752 - RNA, 21 nt, synthetic construct
  misc_feature 1..21: note = artificial sequence
  source 1..21: mol_type = other RNA
accgcattttctcctggcgtt

SEQ ID NO: 753 - RNA, 21 nt, synthetic construct
  misc_feature 1..21: note = artificial sequence
  source 1..21: mol_type = other RNA
gagcatcacggtgcgcacgaa

SEQ ID NO: 754 - RNA, 21 nt, synthetic construct
  misc_feature 1..21: note = artificial sequence
  source 1..21: mol_type = other RNA
ttcgggatccaggatggcgaa

SEQ ID NO: 755 - RNA, 21 nt, synthetic construct
  misc_feature 1..21: note = artificial sequence
  source 1..21: mol_type = other RNA
tggtgacaatgtaccatgcta

SEQ ID NO: 756 - RNA, 21 nt, synthetic construct
  misc_feature 1..21: note = artificial sequence
  source 1..21: mol_type = other RNA
gtgtctggagttctttgccaa

SEQ ID NO: 757 - RNA, 21 nt, synthetic construct
  misc_feature 1..21: note = artificial sequence
  source 1..21: mol_type = other RNA
ccggttctaccatccggaaaa

SEQ ID NO: 758 - RNA, 21 nt, synthetic construct
  misc_feature 1..21: note = artificial sequence
  source 1..21: mol_type = other RNA
tcgtgccggaaggaatcagaa

SEQ ID NO: 759 - RNA, 21 nt, synthetic construct
  misc_feature 1..21: note = artificial sequence
  source 1..21: mol_type = other RNA
cagataaagcttcagttatat

SEQ ID NO: 760 - RNA, 21 nt, synthetic construct
  misc_feature 1..21: note = artificial sequence
  source 1..21: mol_type = other RNA
caagatccgctactacaccta

SEQ ID NO: 761 - RNA, 21 nt, synthetic construct
  misc_feature 1..21: note = artificial sequence
  source 1..21: mol_type = other RNA
caggtaccggggagaccagga

SEQ ID NO: 762 - RNA, 21 nt, synthetic construct
  misc_feature 1..21: note = artificial sequence
  source 1..21: mol_type = other RNA
cagcacctggctgaccgccta

SEQ ID NO: 763 - RNA, 21 nt, synthetic construct
  misc_feature 1..21: note = artificial sequence
  source 1..21: mol_type = other RNA
aaccttgatgtgtccctccaa

SEQ ID NO: 764 - RNA, 21 nt, synthetic construct
  misc_feature 1..21: note = artificial sequence
  source 1..21: mol_type = other RNA
aaaggaggatggaaagctgaa

SEQ ID NO: 765 - RNA, 21 nt, synthetic construct
  misc_feature 1..21: note = artificial sequence
  source 1..21: mol_type = other RNA
ccctggtaagcagctctacaa

SEQ ID NO: 766 - RNA, 21 nt, synthetic construct
  misc_feature 1..21: note = artificial sequence
  source 1..21: mol_type = other RNA
gggggccttggagctcatcaa

SEQ ID NO: 767 - RNA, 21 nt, synthetic construct
  misc_feature 1..21: note = artificial sequence
  source 1..21: mol_type = other RNA
ttcgaaacgagcaggtggaaa

SEQ ID NO: 768 - RNA, 21 nt, synthetic construct
  misc_feature 1..21: note = artificial sequence
  source 1..21: mol_type = other RNA
atccggaaaaggaggatggaa

SEQ ID NO: 769 - RNA, 21 nt, synthetic construct
  misc_feature 1..21: note = artificial sequence
  source 1..21: mol_type = other RNA
ggccaatggtgttgacagata

SEQ ID NO: 770 - RNA, 21 nt, synthetic construct
  misc_feature 1..21: note = artificial sequence
  source 1..21: mol_type = other RNA
tggaacgttgaggacttgaaa

SEQ ID NO: 771 - RNA, 21 nt, synthetic construct
  misc_feature 1..21: note = artificial sequence
  source 1..21: mol_type = other RNA
gaggatgccgtcgacgcggaa

SEQ ID NO: 772 - RNA, 21 nt, synthetic construct
  misc_feature 1..21: note = artificial sequence
  source 1..21: mol_type = other RNA
aggagacttccttgaagccaa

SEQ ID NO: 773 - RNA, 21 nt, synthetic construct
  misc_feature 1..21: note = artificial sequence
  source 1..21: mol_type = other RNA
gacttgaaagagccaccgaaa

SEQ ID NO: 774 - RNA, 21 nt, synthetic construct
  misc_feature 1..21: note = artificial sequence
  source 1..21: mol_type = other RNA
acgaagctcatgaatatattt

SEQ ID NO: 775 - RNA, 21 nt, synthetic construct
  misc_feature 1..21: note = artificial sequence
  source 1..21: mol_type = other RNA
gctcatgaatatatttttgaa

SEQ ID NO: 776 - RNA, 21 nt, synthetic construct
  misc_feature 1..21: note = artificial sequence
  source 1..21: mol_type = other RNA
atagtggagcctacagagaaa

SEQ ID NO: 777 - RNA, 21 nt, synthetic construct
  misc_feature 1..21: note = artificial sequence
  source 1..21: mol_type = other RNA
catgaatatatttttgaaaga

SEQ ID NO: 778 - RNA, 21 nt, synthetic construct
  misc_feature 1..21: note = artificial sequence
  source 1..21: mol_type = other RNA
caccttgtcggtggtgacaat

SEQ ID NO: 779 - RNA, 21 nt, synthetic construct
  misc_feature 1..21: note = artificial sequence
  source 1..21: mol_type = other RNA
gtccacgacttcccaggcaaa

SEQ ID NO: 780 - RNA, 21 nt, synthetic construct
  misc_feature 1..21: note = artificial sequence
  source 1..21: mol_type = other RNA
ccggggagaccctcaacgtca

SEQ ID NO: 781 - RNA, 21 nt, synthetic construct
  misc_feature 1..21: note = artificial sequence
  source 1..21: mol_type = other RNA
accaggatgccactatgtcta

SEQ ID NO: 782 - RNA, 21 nt, synthetic construct
  misc_feature 1..21: note = artificial sequence
  source 1..21: mol_type = other RNA
aacggagcagtgggagaagtt

SEQ ID NO: 783 - RNA, 21 nt, synthetic construct
  misc_feature 1..21: note = artificial sequence
  source 1..21: mol_type = other RNA
atcaactcacctgtaataaat

SEQ ID NO: 784 - RNA, 21 nt, synthetic construct
  misc_feature 1..21: note = artificial sequence
  source 1..21: mol_type = other RNA
aagctcatgaatatatttttg

SEQ ID NO: 785 - RNA, 21 nt, synthetic construct
  misc_feature 1..21: note = artificial sequence
  source 1..21: mol_type = other RNA
cgtgttcgtgctgaataagaa

SEQ ID NO: 786 - RNA, 21 nt, synthetic construct
  misc_feature 1..21: note = artificial sequence
  source 1..21: mol_type = other RNA
gtcgtgcgttggctcaatgaa

SEQ ID NO: 787 - RNA, 21 nt, synthetic construct
  misc_feature 1..21: note = artificial sequence
  source 1..21: mol_type = other RNA
cagagaaattctactacatct

SEQ ID NO: 788 - RNA, 21 nt, synthetic construct
  misc_feature 1..21: note = artificial sequence
  source 1..21: mol_type = other RNA
cccactccagataaagcttca

SEQ ID NO: 789 - RNA, 21 nt, synthetic construct
  misc_feature 1..21: note = artificial sequence
  source 1..21: mol_type = other RNA
acagtcacagctgaaggaaaa

SEQ ID NO: 790 - RNA, 21 nt, synthetic construct
  misc_feature 1..21: note = artificial sequence
  source 1..21: mol_type = other RNA
ttggacagcagcgcacgttca

SEQ ID NO: 791 - RNA, 21 nt, synthetic construct
  misc_feature 1..21: note = artificial sequence
  source 1..21: mol_type = other RNA
caaggttcagctgtccaatga

SEQ ID NO: 792 - RNA, 21 nt, synthetic construct
  misc_feature 1..21: note = artificial sequence
  source 1..21: mol_type = other RNA
caaagatcaactcacctgtaa

SEQ ID NO: 793 - RNA, 21 nt, synthetic construct
  misc_feature 1..21: note = artificial sequence
  source 1..21: mol_type = other RNA
tccagataaagcttcagttat

SEQ ID NO: 794 - RNA, 21 nt, synthetic construct
  misc_feature 1..21: note = artificial sequence
  source 1..21: mol_type = other RNA
ctgtctagctttcaaagttca

SEQ ID NO: 795 - RNA, 21 nt, synthetic construct
  misc_feature 1..21: note = artificial sequence
  source 1..21: mol_type = other RNA
atcgctgtgcattacctggat

SEQ ID NO: 796 - RNA, 21 nt, synthetic construct
  misc_feature 1..21: note = artificial sequence
  source 1..21: mol_type = other RNA
ggagaattgcttcatacaaaa

SEQ ID NO: 797 - RNA, 21 nt, synthetic construct
  misc_feature 1..21: note = artificial sequence
  source 1..21: mol_type = other RNA
gaggcctcaggatgccaagaa

SEQ ID NO: 798 - RNA, 21 nt, synthetic construct
  misc_feature 1..21: note = artificial sequence
  source 1..21: mol_type = other RNA
cagcatcactaaagcaggaga

SEQ ID NO: 799 - RNA, 21 nt, synthetic construct
  misc_feature 1..21: note = artificial sequence
  source 1..21: mol_type = other RNA
aagatccgagcctactatgaa

SEQ ID NO: 800 - RNA, 21 nt, synthetic construct
  misc_feature 1..21: note = artificial sequence
  source 1..21: mol_type = other RNA
cgctgcaggaggctaaagata

SEQ ID NO: 801 - RNA, 21 nt, synthetic construct
  misc_feature 1..21: note = artificial sequence
  source 1..21: mol_type = other RNA
tggcgttcgcctgctagtgtg

SEQ ID NO: 802 - RNA, 21 nt, synthetic construct
  misc_feature 1..21: note = artificial sequence
  source 1..21: mol_type = other RNA
cctgcaggaagtggaagtcaa

SEQ ID NO: 803 - RNA, 21 nt, synthetic construct
  misc_feature 1..21: note = artificial sequence
  source 1..21: mol_type = other RNA
cccagagagctggctgtggaa

SEQ ID NO: 804 - RNA, 21 nt, synthetic construct
  misc_feature 1..21: note = artificial sequence
  source 1..21: mol_type = other RNA
ctgccaggcagcatcactaaa

SEQ ID NO: 805 - RNA, 21 nt, synthetic construct
  misc_feature 1..21: note = artificial sequence
  source 1..21: mol_type = other RNA
cctcagctacatcatcgggaa

SEQ ID NO: 806 - RNA, 21 nt, synthetic construct
  misc_feature 1..21: note = artificial sequence
  source 1..21: mol_type = other RNA
ttgcttcatacaaaagtcgga

SEQ ID NO: 807 - RNA, 21 nt, synthetic construct
  misc_feature 1..21: note = artificial sequence
  source 1..21: mol_type = other RNA
ctggacaaggcctgtgagcca

SEQ ID NO: 808 - RNA, 21 nt, synthetic construct
  misc_feature 1..21: note = artificial sequence
  source 1..21: mol_type = other RNA
aaggcaccttgtcggtggtga

SEQ ID NO: 809 - RNA, 21 nt, synthetic construct
  misc_feature 1..21: note = artificial sequence
  source 1..21: mol_type = other RNA
tggtggattacggaacaacaa

SEQ ID NO: 810 - RNA, 21 nt, synthetic construct
  misc_feature 1..21: note = artificial sequence
  source 1..21: mol_type = other RNA
ctgtgagccaggagtggacta

SEQ ID NO: 811 - RNA, 21 nt, synthetic construct
  misc_feature 1..21: note = artificial sequence
  source 1..21: mol_type = other RNA
ctccacagttctctatcggat

SEQ ID NO: 812 - RNA, 21 nt, synthetic construct
  misc_feature 1..21: note = artificial sequence
  source 1..21: mol_type = other RNA
cagctcagctccatcctactt

SEQ ID NO: 813 - RNA, 21 nt, synthetic construct
  misc_feature 1..21: note = artificial sequence
  source 1..21: mol_type = other RNA
tcggatgaggtgcaggttgga

SEQ ID NO: 814 - RNA, 21 nt, synthetic construct
  misc_feature 1..21: note = artificial sequence
  source 1..21: mol_type = other RNA
aagcagctggccaatggtgtt

SEQ ID NO: 815 - RNA, 21 nt, synthetic construct
  misc_feature 1..21: note = artificial sequence
  source 1..21: mol_type = other RNA
aactacatgaacctacagaga

SEQ ID NO: 816 - RNA, 21 nt, synthetic construct
  misc_feature 1..21: note = artificial sequence
  source 1..21: mol_type = other RNA
acgaagagaaccagaaacaat

SEQ ID NO: 817 - RNA, 21 nt, synthetic construct
  misc_feature 1..21: note = artificial sequence
  source 1..21: mol_type = other RNA
gagctcaaggtgagggtggaa

SEQ ID NO: 818 - RNA, 21 nt, synthetic construct
  misc_feature 1..21: note = artificial sequence
  source 1..21: mol_type = other RNA
caggtcttctccactgagttt

SEQ ID NO: 819 - RNA, 21 nt, synthetic construct
  misc_feature 1..21: note = artificial sequence
  source 1..21: mol_type = other RNA
aagagaacatcgtttcccgaa

SEQ ID NO: 820 - RNA, 21 nt, synthetic construct
  misc_feature 1..21: note = artificial sequence
  source 1..21: mol_type = other RNA
ttcgacctcaaggtcaccata

SEQ ID NO: 821 - RNA, 21 nt, synthetic construct
  misc_feature 1..21: note = artificial sequence
  source 1..21: mol_type = other RNA
catctcgctgcaggaggctaa

SEQ ID NO: 822 - RNA, 21 nt, synthetic construct
  misc_feature 1..21: note = artificial sequence
  source 1..21: mol_type = other RNA
gtggaagatccgagcctacta

SEQ ID NO: 823 - RNA, 21 nt, synthetic construct
  misc_feature 1..21: note = artificial sequence
  source 1..21: mol_type = other RNA
aggggcctcttcttaacaaat

SEQ ID NO: 824 - RNA, 21 nt, synthetic construct
  misc_feature 1..21: note = artificial sequence
  source 1..21: mol_type = other RNA
cttgtcggtggtgacaatgta

SEQ ID NO: 825 - RNA, 21 nt, synthetic construct
  misc_feature 1..21: note = artificial sequence
  source 1..21: mol_type = other RNA
aacctggatgaggacatcatt

SEQ ID NO: 826 - RNA, 21 nt, synthetic construct
  misc_feature 1..21: note = artificial sequence
  source 1..21: mol_type = other RNA
tccgagccgttctctacaatt

SEQ ID NO: 827 - RNA, 21 nt, synthetic construct
  misc_feature 1..21: note = artificial sequence
  source 1..21: mol_type = other RNA
caccataaaaccagcaccgga

SEQ ID NO: 828 - RNA, 21 nt, synthetic construct
  misc_feature 1..21: note = artificial sequence
  source 1..21: mol_type = other RNA
gggccggacggtcatggtcaa

SEQ ID NO: 829 - RNA, 21 nt, synthetic construct
  misc_feature 1..21: note = artificial sequence
  source 1..21: mol_type = other RNA
gacggtcatggtcaacattga

SEQ ID NO: 830 - RNA, 21 nt, synthetic construct
  misc_feature 1..21: note = artificial sequence
  source 1..21: mol_type = other RNA
ccgctgggaggaccctggtaa

SEQ ID NO: 831 - RNA, 21 nt, synthetic construct
  misc_feature 1..21: note = artificial sequence
  source 1..21: mol_type = other RNA
cgcccgtgatacaccaagaaa

SEQ ID NO: 832 - RNA, 21 nt, synthetic construct
  misc_feature 1..21: note = artificial sequence
  source 1..21: mol_type = other RNA
cctgaagctggaggagaagaa

SEQ ID NO: 833 - RNA, 21 nt, synthetic construct
  misc_feature 1..21: note = artificial sequence
  source 1..21: mol_type = other RNA
ctccctgggcgaggcgtgcaa

SEQ ID NO: 834 - RNA, 21 nt, synthetic construct
  misc_feature 1..21: note = artificial sequence
  source 1..21: mol_type = other RNA
cactatgtctatattggacat

SEQ ID NO: 835 - RNA, 21 nt, synthetic construct
  misc_feature 1..21: note = artificial sequence
  source 1..21: mol_type = other RNA
ttgcagaagagaacatcgttt

SEQ ID NO: 836 - RNA, 21 nt, synthetic construct
  misc_feature 1..21: note = artificial sequence
  source 1..21: mol_type = other RNA
gagcgggtacctcttcatcca

SEQ ID NO: 837 - RNA, 21 nt, synthetic construct
  misc_feature 1..21: note = artificial sequence
  source 1..21: mol_type = other RNA
cagctctgcctttgcggcctt

SEQ ID NO: 838 - RNA, 21 nt, synthetic construct
  misc_feature 1..21: note = artificial sequence
  source 1..21: mol_type = other RNA
caggaatgccctttgacctca

SEQ ID NO: 839 - RNA, 21 nt, synthetic construct
  misc_feature 1..21: note = artificial sequence
  source 1..21: mol_type = other RNA
ccccatcgtgacctctcccta

SEQ ID NO: 840 - RNA, 21 nt, synthetic construct
  misc_feature 1..21: note = artificial sequence
  source 1..21: mol_type = other RNA
gctgaggagaattgcttcata

SEQ ID NO: 841 - RNA, 21 nt, synthetic construct
  misc_feature 1..21: note = artificial sequence
  source 1..21: mol_type = other RNA
aacaagctctgccgtgatgaa

SEQ ID NO: 842 - RNA, 21 nt, synthetic construct
  misc_feature 1..21: note = artificial sequence
  source 1..21: mol_type = other RNA
cggaaggaatcagaatgaaca

SEQ ID NO: 843 - RNA, 21 nt, synthetic construct
  misc_feature 1..21: note = artificial sequence
  source 1..21: mol_type = other RNA
atcagaagagaccaaggaaaa

SEQ ID NO: 844 - RNA, 21 nt, synthetic construct
  misc_feature 1..21: note = artificial sequence
  source 1..21: mol_type = other RNA
ctggacaaggtctcacactct

SEQ ID NO: 845 - RNA, 21 nt, synthetic construct
  misc_feature 1..21: note = artificial sequence
  source 1..21: mol_type = other RNA
ctgcagcctggccaccaccaa

SEQ ID NO: 846 - RNA, 21 nt, synthetic construct
  misc_feature 1..21: note = artificial sequence
  source 1..21: mol_type = other RNA
aggagtggactatgtgtacaa

SEQ ID NO: 847 - RNA, 21 nt, synthetic construct
  misc_feature 1..21: note = artificial sequence
  source 1..21: mol_type = other RNA
ctggcgttcgcctgctagtgt

SEQ ID NO: 848 - RNA, 21 nt, synthetic construct
  misc_feature 1..21: note = artificial sequence
  source 1..21: mol_type = other RNA
catcagtgacggtgtcaggaa

SEQ ID NO: 849 - RNA, 21 nt, synthetic construct
  misc_feature 1..21: note = artificial sequence
  source 1..21: mol_type = other RNA
ctacagagaaattctactaca

SEQ ID NO: 850 - RNA, 21 nt, synthetic construct
  misc_feature 1..21: note = artificial sequence
  source 1..21: mol_type = other RNA
aacagaaaagaggcctcagga

SEQ ID NO: 851 - RNA, 21 nt, synthetic construct
  misc_feature 1..21: note = artificial sequence
  source 1..21: mol_type = other RNA
ctccgatttctggggagagaa

SEQ ID NO: 852 - RNA, 21 nt, synthetic construct
  misc_feature 1..21: note = artificial sequence
  source 1..21: mol_type = other RNA
cctcatggtgttcgtgacgaa

SEQ ID NO: 853 - RNA, 21 nt, synthetic construct
  misc_feature 1..21: note = artificial sequence
  source 1..21: mol_type = other RNA
ctgtgctgaggagaattgctt

SEQ ID NO: 854 - RNA, 21 nt, synthetic construct
  misc_feature 1..21: note = artificial sequence
  source 1..21: mol_type = other RNA
gccaggagtggactatgtgta

SEQ ID NO: 855 - RNA, 21 nt, synthetic construct
  misc_feature 1..21: note = artificial sequence
  source 1..21: mol_type = other RNA
cagggcgaggacactgtgcag

SEQ ID NO: 856 - RNA, 21 nt, synthetic construct
  misc_feature 1..21: note = artificial sequence
  source 1..21: mol_type = other RNA
ccctgaccaccaggaactgaa

SEQ ID NO: 857 - RNA, 21 nt, synthetic construct
  misc_feature 1..21: note = artificial sequence
  source 1..21: mol_type = other RNA
cagtttcgaggtcatagtgga

SEQ ID NO: 858 - RNA, 21 nt, synthetic construct
  misc_feature 1..21: note = artificial sequence
  source 1..21: mol_type = other RNA
agccaactacatgaacctaca

SEQ ID NO: 859 - RNA, 21 nt, synthetic construct
  misc_feature 1..21: note = artificial sequence
  source 1..21: mol_type = other RNA
gcgggtacctcttcatccaga

SEQ ID NO: 860 - RNA, 21 nt, synthetic construct
  misc_feature 1..21: note = artificial sequence
  source 1..21: mol_type = other RNA
ctcgctgcaggaggctaaaga

SEQ ID NO: 861 - RNA, 21 nt, synthetic construct
  misc_feature 1..21: note = artificial sequence
  source 1..21: mol_type = other RNA
gcgaatggacaaagtcggcaa

SEQ ID NO: 862 - RNA, 21 nt, synthetic construct
  misc_feature 1..21: note = artificial sequence
  source 1..21: mol_type = other RNA
cacgtgggagattctggctgt

SEQ ID NO: 863 - RNA, 21 nt, synthetic construct
  misc_feature 1..21: note = artificial sequence
  source 1..21: mol_type = other RNA
caccaagaaatgattggtgga

SEQ ID NO: 864 - RNA, 21 nt, synthetic construct
  misc_feature 1..21: note = artificial sequence
  source 1..21: mol_type = other RNA
taccctactctgttgttcgaa

SEQ ID NO: 865 - RNA, 21 nt, synthetic construct
  misc_feature 1..21: note = artificial sequence
  source 1..21: mol_type = other RNA
cagcagatgaccctgaagata

SEQ ID NO: 866 - RNA, 21 nt, synthetic construct
  misc_feature 1..21: note = artificial sequence
  source 1..21: mol_type = other RNA
gacttcccaggcaaaaaacta

SEQ ID NO: 867 - RNA, 21 nt, synthetic construct
  misc_feature 1..21: note = artificial sequence
  source 1..21: mol_type = other RNA
tggggagtcccatgtactcta

SEQ ID NO: 868 - RNA, 21 nt, synthetic construct
  misc_feature 1..21: note = artificial sequence
  source 1..21: mol_type = other RNA
gaaggggcctcttcttaacaa

SEQ ID NO: 869 - RNA, 21 nt, synthetic construct
  misc_feature 1..21: note = artificial sequence
  source 1..21: mol_type = other RNA
caggatggcgaacagaggatt

SEQ ID NO: 870 - RNA, 21 nt, synthetic construct
  misc_feature 1..21: note = artificial sequence
  source 1..21: mol_type = other RNA
ccgctactacacctacctgat

SEQ ID NO: 871 - RNA, 21 nt, synthetic construct
  misc_feature 1..21: note = artificial sequence
  source 1..21: mol_type = other RNA
ccctggctccacagttctcta

SEQ ID NO: 872 - RNA, 21 nt, synthetic construct
  misc_feature 1..21: note = artificial sequence
  source 1..21: mol_type = other RNA
aagcgcattccgattgaggat

SEQ ID NO: 873 - RNA, 21 nt, synthetic construct
  misc_feature 1..21: note = artificial sequence
  source 1..21: mol_type = other RNA
aaagatcaactcacctgtaat

SEQ ID NO: 874 - RNA, 21 nt, synthetic construct
  misc_feature 1..21: note = artificial sequence
  source 1..21: mol_type = other RNA
tcagctccatcctacttgaaa

SEQ ID NO: 875 - RNA, 21 nt, synthetic construct
  misc_feature 1..21: note = artificial sequence
  source 1..21: mol_type = other RNA
catgctaaggccaaagatcaa

SEQ ID NO: 876 - RNA, 21 nt, synthetic construct
  misc_feature 1..21: note = artificial sequence
  source 1..21: mol_type = other RNA
cttggccctactgcagctaaa

SEQ ID NO: 877 - RNA, 21 nt, synthetic construct
  misc_feature 1..21: note = artificial sequence
  source 1..21: mol_type = other RNA
ttggccctactgcagctaaaa

SEQ ID NO: 878 - RNA, 21 nt, synthetic construct
  misc_feature 1..21: note = artificial sequence
  source 1..21: mol_type = other RNA
ccgtgaaggagtgcagaaaga

SEQ ID NO: 879 - RNA, 21 nt, synthetic construct
  misc_feature 1..21: note = artificial sequence
  source 1..21: mol_type = other RNA
cagttctctatcggatcttca

SEQ ID NO: 880 - RNA, 21 nt, synthetic construct
  misc_feature 1..21: note = artificial sequence
  source 1..21: mol_type = other RNA
caggttcctctacgggaagaa

SEQ ID NO: 881 - RNA, 21 nt, synthetic construct
  misc_feature 1..21: note = artificial sequence
  source 1..21: mol_type = other RNA
gaggtgcaggttggacagcag

SEQ ID NO: 882 - RNA, 21 nt, synthetic construct
  misc_feature 1..21: note = artificial sequence
  source 1..21: mol_type = other RNA
tgccggaaggaatcagaatga

SEQ ID NO: 883 - RNA, 21 nt, synthetic construct
  misc_feature 1..21: note = artificial sequence
  source 1..21: mol_type = other RNA
cgccaggttcctctacgggaa

SEQ ID NO: 884 - RNA, 21 nt, synthetic construct
  misc_feature 1..21: note = artificial sequence
  source 1..21: mol_type = other RNA
gaggacactgtgcagtctcta

SEQ ID NO: 885 - RNA, 21 nt, synthetic construct
  misc_feature 1..21: note = artificial sequence
  source 1..21: mol_type = other RNA
caaagttcaccaatactttaa

SEQ ID NO: 886 - RNA, 21 nt, synthetic construct
  misc_feature 1..21: note = artificial sequence
  source 1..21: mol_type = other RNA
ccgtgggcaactccaacaatt

SEQ ID NO: 887 - RNA, 21 nt, synthetic construct
  misc_feature 1..21: note = artificial sequence
  source 1..21: mol_type = other RNA
aagaaagggatctgtgtggca

SEQ ID NO: 888 - RNA, 21 nt, synthetic construct
  misc_feature 1..21: note = artificial sequence
  source 1..21: mol_type = other RNA
aagacacccaagtacttcaaa

SEQ ID NO: 889 - RNA, 21 nt, synthetic construct
  misc_feature 1..21: note = artificial sequence
  source 1..21: mol_type = other RNA
cactgtggccattgctggcta

SEQ ID NO: 890 - RNA, 21 nt, synthetic construct
  misc_feature 1..21: note = artificial sequence
  source 1..21: mol_type = other RNA
caagacacccaagtacttcaa

SEQ ID NO: 891 - RNA, 21 nt, synthetic construct
  misc_feature 1..21: note = artificial sequence
  source 1..21: mol_type = other RNA
tgcagctcacggagaagcgaa

SEQ ID NO: 892 - RNA, 21 nt, synthetic construct
  misc_feature 1..21: note = artificial sequence
  source 1..21: mol_type = other RNA
gaagctcatgaatatattttt

SEQ ID NO: 893 - RNA, 21 nt, synthetic construct
  misc_feature 1..21: note = artificial sequence
  source 1..21: mol_type = other RNA
atgtaccatgctaaggccaaa

SEQ ID NO: 894 - RNA, 21 nt, synthetic construct
  misc_feature 1..21: note = artificial sequence
  source 1..21: mol_type = other RNA
cccagccaacagggagttcaa

SEQ ID NO: 895 - RNA, 21 nt, synthetic construct
  misc_feature 1..21: note = artificial sequence
  source 1..21: mol_type = other RNA
ctgtgcattacctggatgaaa

SEQ ID NO: 896 - RNA, 21 nt, synthetic construct
  misc_feature 1..21: note = artificial sequence
  source 1..21: mol_type = other RNA
aacagggagttcaagtcagaa

SEQ ID NO: 897 - RNA, 21 nt, synthetic construct
  misc_feature 1..21: note = artificial sequence
  source 1..21: mol_type = other RNA
acagggagttcaagtcagaaa

SEQ ID NO: 898 - RNA, 21 nt, synthetic construct
  misc_feature 1..21: note = artificial sequence
  source 1..21: mol_type = other RNA
cttgaaagagccaccgaaaaa

SEQ ID NO: 899 - RNA, 21 nt, synthetic construct
  misc_feature 1..21: note = artificial sequence
  source 1..21: mol_type = other RNA
cccccactccagataaagctt

SEQ ID NO: 900 - RNA, 21 nt, synthetic construct
  misc_feature 1..21: note = artificial sequence
  source 1..21: mol_type = other RNA
gtcgtgccggaaggaatcaga

SEQ ID NO: 901 - RNA, 21 nt, synthetic construct
  misc_feature 1..21: note = artificial sequence
  source 1..21: mol_type = other RNA
ctccaagatcacccaccgtat

SEQ ID NO: 902 - RNA, 21 nt, synthetic construct
  misc_feature 1..21: note = artificial sequence
  source 1..21: mol_type = other RNA
ttccttccgcctggtggcgta

SEQ ID NO: 903 - RNA, 21 nt, synthetic construct
  misc_feature 1..21: note = artificial sequence
  source 1..21: mol_type = other RNA
agagaaattctactacatcta

SEQ ID NO: 904 - RNA, 21 nt, synthetic construct
  misc_feature 1..21: note = artificial sequence
  source 1..21: mol_type = other RNA
caggagacttccttgaagcca

SEQ ID NO: 905 - RNA, 21 nt, synthetic construct
  misc_feature 1..21: note = artificial sequence
  source 1..21: mol_type = other RNA
tcctggactgctgcaactaca

SEQ ID NO: 906 - RNA, 21 nt, synthetic construct
  misc_feature 1..21: note = artificial sequence
  source 1..21: mol_type = other RNA
ccggaaggaatcagaatgaac

SEQ ID NO: 907 - RNA, 21 nt, synthetic construct
  misc_feature 1..21: note = artificial sequence
  source 1..21: mol_type = other RNA
aaaccagcaccggaaacagaa

SEQ ID NO: 908 - RNA, 21 nt, synthetic construct
  misc_feature 1..21: note = artificial sequence
  source 1..21: mol_type = other RNA
ctgcggggaacagaacatgat

SEQ ID NO: 909 - RNA, 21 nt, synthetic construct
  misc_feature 1..21: note = artificial sequence
  source 1..21: mol_type = other RNA
ttgccaagagggagaggctga

SEQ ID NO: 910 - RNA, 21 nt, synthetic construct
  misc_feature 1..21: note = artificial sequence
  source 1..21: mol_type = other RNA
tgagtttgaggtgaaggagta

SEQ ID NO: 911 - RNA, 21 nt, synthetic construct
  misc_feature 1..21: note = artificial sequence
  source 1..21: mol_type = other RNA
ctgcggggctgttaaatggct

SEQ ID NO: 912 - RNA, 21 nt, synthetic construct
  misc_feature 1..21: note = artificial sequence
  source 1..21: mol_type = other RNA
gtgctgaataagaagaacaaa

SEQ ID NO: 913 - RNA, 21 nt, synthetic construct
  misc_feature 1..21: note = artificial sequence
  source 1..21: mol_type = other RNA
atccgagccgttctctacaat

SEQ ID NO: 914 - RNA, 21 nt, synthetic construct
  misc_feature 1..21: note = artificial sequence
  source 1..21: mol_type = other RNA
taaatggctgatcctggagaa

SEQ ID NO: 915 - RNA, 21 nt, synthetic construct
  misc_feature 1..21: note = artificial sequence
  source 1..21: mol_type = other RNA
cctcatcatctacctggacaa

SEQ ID NO: 916 - RNA, 21 nt, synthetic construct
  misc_feature 1..21: note = artificial sequence
  source 1..21: mol_type = other RNA
aaggagtgcagaaagaggaca

SEQ ID NO: 917 - RNA, 21 nt, synthetic construct
  misc_feature 1..21: note = artificial sequence
  source 1..21: mol_type = other RNA
caccaagaggcgtcaccagca

SEQ ID NO: 918 - RNA, 21 nt, synthetic construct
  misc_feature 1..21: note = artificial sequence
  source 1..21: mol_type = other RNA
gcgcccgtgatacaccaagaa

SEQ ID NO: 919 - RNA, 21 nt, synthetic construct
  misc_feature 1..21: note = artificial sequence
  source 1..21: mol_type = other RNA
gcggggctgttaaatggctga

SEQ ID NO: 920 - RNA, 21 nt, synthetic construct
  misc_feature 1..21: note = artificial sequence
  source 1..21: mol_type = other RNA
gtcctctgcggggctgttaaa

SEQ ID NO: 921 - RNA, 21 nt, synthetic construct
  misc_feature 1..21: note = artificial sequence
  source 1..21: mol_type = other RNA
taccatgctaaggccaaagat

SEQ ID NO: 922 - RNA, 21 nt, synthetic construct
  misc_feature 1..21: note = artificial sequence
  source 1..21: mol_type = other RNA
ctacacctacctgatcatgaa

SEQ ID NO: 923 - RNA, 21 nt, synthetic construct
  misc_feature 1..21: note = artificial sequence
  source 1..21: mol_type = other RNA
cagatacatctccaagtatga

SEQ ID NO: 924 - RNA, 21 nt, synthetic construct
  misc_feature 1..21: note = artificial sequence
  source 1..21: mol_type = other RNA
cacagtaatgcaggacttctt

SEQ ID NO: 925 - RNA, 21 nt, synthetic construct
  misc_feature 1..21: note = artificial sequence
  source 1..21: mol_type = other RNA
acctcacctgttcccaccgca

SEQ ID NO: 926 - RNA, 21 nt, synthetic construct
  misc_feature 1..21: note = artificial sequence
  source 1..21: mol_type = other RNA
tccagacacagatgacctgaa

SEQ ID NO: 927 - RNA, 21 nt, synthetic construct
  misc_feature 1..21: note = artificial sequence
  source 1..21: mol_type = other RNA
tgggccggacggtcatggtca

SEQ ID NO: 928 - RNA, 21 nt, synthetic construct
  misc_feature 1..21: note = artificial sequence
  source 1..21: mol_type = other RNA
acagaaaagaggcctcaggat

SEQ ID NO: 929 - RNA, 21 nt, synthetic construct
  misc_feature 1..21: note = artificial sequence
  source 1..21: mol_type = other RNA
gatgactgtctagctttcaaa

SEQ ID NO: 930 - RNA, 21 nt, synthetic construct
  misc_feature 1..21: note = artificial sequence
  source 1..21: mol_type = other RNA
gactgtctagctttcaaagtt

SEQ ID NO: 931 - RNA, 21 nt, synthetic construct
  misc_feature 1..21: note = artificial sequence
  source 1..21: mol_type = other RNA
tcgaggtcatagtggagccta

SEQ ID NO: 932 - RNA, 21 nt, synthetic construct
  misc_feature 1..21: note = artificial sequence
  source 1..21: mol_type = other RNA
gaccgtaaccatcccccccaa

SEQ ID NO: 933 - RNA, 21 nt, synthetic construct
  misc_feature 1..21: note = artificial sequence
  source 1..21: mol_type = other RNA
aaggttcagctgtccaatgac

SEQ ID NO: 934 - RNA, 21 nt, synthetic construct
  misc_feature 1..21: note = artificial sequence
  source 1..21: mol_type = other RNA
aatctctacgaagctcatgaa

SEQ ID NO: 935 - RNA, 21 nt, synthetic construct
  misc_feature 1..21: note = artificial sequence
  source 1..21: mol_type = other RNA
tcggtggtgacaatgtaccat

SEQ ID NO: 936 - RNA, 21 nt, synthetic construct
  misc_feature 1..21: note = artificial sequence
  source 1..21: mol_type = other RNA
agggggccttggagctcatca

SEQ ID NO: 937 - RNA, 21 nt, synthetic construct
  misc_feature 1..21: note = artificial sequence
  source 1..21: mol_type = other RNA
accagcaccggaaacagaaaa

SEQ ID NO: 938 - RNA, 21 nt, synthetic construct
  misc_feature 1..21: note = artificial sequence
  source 1..21: mol_type = other RNA
cagcaccggaaacagaaaaga

SEQ ID NO: 939 - RNA, 21 nt, synthetic construct
  misc_feature 1..21: note = artificial sequence
  source 1..21: mol_type = other RNA
ccaagacgaagagaaccagaa

SEQ ID NO: 940 - RNA, 21 nt, synthetic construct
  misc_feature 1..21: note = artificial sequence
  source 1..21: mol_type = other RNA
ccggaaaaggaggatggaaag

SEQ ID NO: 941 - RNA, 21 nt, synthetic construct
  misc_feature 1..21: note = artificial sequence
  source 1..21: mol_type = other RNA
caaggtgagggtggaactact

SEQ ID NO: 942 - RNA, 21 nt, synthetic construct
  misc_feature 1..21: note = artificial sequence
  source 1..21: mol_type = other RNA
tccctcaagcgcattccgatt

SEQ ID NO: 943 - RNA, 21 nt, synthetic construct
  misc_feature 1..21: note = artificial sequence
  source 1..21: mol_type = other RNA
accgctgggaggaccctggta

SEQ ID NO: 944 - RNA, 21 nt, synthetic construct
  misc_feature 1..21: note = artificial sequence
  source 1..21: mol_type = other RNA
caccaggaactgaaccttgat

SEQ ID NO: 945 - RNA, 21 nt, synthetic construct
  misc_feature 1..21: note = artificial sequence
  source 1..21: mol_type = other RNA
cccggttctaccatccggaaa

SEQ ID NO: 946 - RNA, 21 nt, synthetic construct
  misc_feature 1..21: note = artificial sequence
  source 1..21: mol_type = other RNA
ccagggagatggcgtggccaa

SEQ ID NO: 947 - RNA, 21 nt, synthetic construct
  misc_feature 1..21: note = artificial sequence
  source 1..21: mol_type = other RNA
ttccgtgcagctcacggagaa

SEQ ID NO: 948 - RNA, 21 nt, synthetic construct
  misc_feature 1..21: note = artificial sequence
  source 1..21: mol_type = other RNA
cccattcccccactccagata

SEQ ID NO: 949 - RNA, 21 nt, synthetic construct
  misc_feature 1..21: note = artificial sequence
  source 1..21: mol_type = other RNA
cacgacgcgcaaggggatgtt

SEQ ID NO: 950 - RNA, 21 nt, synthetic construct
  misc_feature 1..21: note = artificial sequence
  source 1..21: mol_type = other RNA
tcggatcttcaccgtcaacca

SEQ ID NO: 951 - RNA, 21 nt, synthetic construct
  misc_feature 1..21: note = artificial sequence
  source 1..21: mol_type = other RNA
gagctggctgtggaacgttga

SEQ ID NO: 952 - RNA, 21 nt, synthetic construct
  misc_feature 1..21: note = artificial sequence
  source 1..21: mol_type = other RNA
caatactttaatgtagagctt

SEQ ID NO: 953 - RNA, 21 nt, synthetic construct
  misc_feature 1..21: note = artificial sequence
  source 1..21: mol_type = other RNA
aagtcctctgcggggctgtta

SEQ ID NO: 954 - RNA, 21 nt, synthetic construct
  misc_feature 1..21: note = artificial sequence
  source 1..21: mol_type = other RNA
tccgctactacacctacctga

SEQ ID NO: 955 - RNA, 21 nt, synthetic construct
  misc_feature 1..21: note = artificial sequence
  source 1..21: mol_type = other RNA
ggggcctcttcttaacaaatt

SEQ ID NO: 956 - RNA, 21 nt, synthetic construct
  misc_feature 1..21: note = artificial sequence
  source 1..21: mol_type = other RNA
cccgaagtgagttcccagaga

SEQ ID NO: 957 - RNA, 21 nt, synthetic construct
  misc_feature 1..21: note = artificial sequence
  source 1..21: mol_type = other RNA
ttctgaccacagccaaagata

SEQ ID NO: 958 - RNA, 21 nt, synthetic construct
  misc_feature 1..21: note = artificial sequence
  source 1..21: mol_type = other RNA
ctcaaggtgagggtggaacta

SEQ ID NO: 959 - RNA, 21 nt, synthetic construct
  misc_feature 1..21: note = artificial sequence
  source 1..21: mol_type = other RNA
ccccgggcagtgggaaggatt

SEQ ID NO: 960 - RNA, 21 nt, synthetic construct
  misc_feature 1..21: note = artificial sequence
  source 1..21: mol_type = other RNA
ccagtggaagatccgagccta

SEQ ID NO: 961 - RNA, 21 nt, synthetic construct
  misc_feature 1..21: note = artificial sequence
  source 1..21: mol_type = other RNA
caccgacttcatcccttcctt

SEQ ID NO: 962 - RNA, 21 nt, synthetic construct
  misc_feature 1..21: note = artificial sequence
  source 1..21: mol_type = other RNA
cccggaaggcatcccggtcaa

SEQ ID NO: 963 - RNA, 21 nt, synthetic construct
  misc_feature 1..21: note = artificial sequence
  source 1..21: mol_type = other RNA
aatggacaaagtcggcaagta

SEQ ID NO: 964 - RNA, 21 nt, synthetic construct
  misc_feature 1..21: note = artificial sequence
  source 1..21: mol_type = other RNA
catcgacctgcggctacccta

SEQ ID NO: 965 - RNA, 21 nt, synthetic construct
  misc_feature 1..21: note = artificial sequence
  source 1..21: mol_type = other RNA
caggcagcatcactaaagcag

SEQ ID NO: 966 - RNA, 21 nt, synthetic construct
  misc_feature 1..21: note = artificial sequence
  source 1..21: mol_type = other RNA
cagtgacatggtgcaggcaga

SEQ ID NO: 967 - RNA, 21 nt, synthetic construct
  misc_feature 1..21: note = artificial sequence
  source 1..21: mol_type = other RNA
cactgagtttgaggtgaagga

SEQ ID NO: 968 - RNA, 21 nt, synthetic construct
  misc_feature 1..21: note = artificial sequence
  source 1..21: mol_type = other RNA
accccttcgaggtcacagtaa

SEQ ID NO: 969 - RNA, 21 nt, synthetic construct
  misc_feature 1..21: note = artificial sequence
  source 1..21: mol_type = other RNA
atctcgctgcaggaggctaaa

SEQ ID NO: 970 - RNA, 21 nt, synthetic construct
  misc_feature 1..21: note = artificial sequence
  source 1..21: mol_type = other RNA
aaagaggcctcaggatgccaa

SEQ ID NO: 971 - RNA, 21 nt, synthetic construct
  misc_feature 1..21: note = artificial sequence
  source 1..21: mol_type = other RNA
caggaagtggaagtcaaggct

SEQ ID NO: 972 - RNA, 21 nt, synthetic construct
  misc_feature 1..21: note = artificial sequence
  source 1..21: mol_type = other RNA
tgccactatgtctatattgga

SEQ ID NO: 973 - RNA, 21 nt, synthetic construct
  misc_feature 1..21: note = artificial sequence
  source 1..21: mol_type = other RNA
ctgaaggaaaaggccaaggca

SEQ ID NO: 974 - RNA, 21 nt, synthetic construct
  misc_feature 1..21: note = artificial sequence
  source 1..21: mol_type = other RNA
cacggcctttgttctcatctc

SEQ ID NO: 975 - RNA, 21 nt, synthetic construct
  misc_feature 1..21: note = artificial sequence
  source 1..21: mol_type = other RNA
ctcccgtcgtgcgttggctca

SEQ ID NO: 976 - RNA, 21 nt, synthetic construct
  misc_feature 1..21: note = artificial sequence
  source 1..21: mol_type = other RNA
acggctggacaaggcctgtga

SEQ ID NO: 977 - RNA, 21 nt, synthetic construct
  misc_feature 1..21: note = artificial sequence
  source 1..21: mol_type = other RNA
gaccacagccaaagataagaa

SEQ ID NO: 978 - RNA, 21 nt, synthetic construct
  misc_feature 1..21: note = artificial sequence
  source 1..21: mol_type = other RNA
cgtgctgaataagaagaacaa

SEQ ID NO: 979 - RNA, 21 nt, synthetic construct
  misc_feature 1..21: note = artificial sequence
  source 1..21: mol_type = other RNA
tctcctggcgttcgcctgcta

SEQ ID NO: 980 - RNA, 21 nt, synthetic construct
  misc_feature 1..21: note = artificial sequence
  source 1..21: mol_type = other RNA
caggctgaaggggcctcttct

SEQ ID NO: 981 - RNA, 21 nt, synthetic construct
  misc_feature 1..21: note = artificial sequence
  source 1..21: mol_type = other RNA
gtgggagaagttcggcctaga

SEQ ID NO: 982 - RNA, 21 nt, synthetic construct
  misc_feature 1..21: note = artificial sequence
  source 1..21: mol_type = other RNA
atgaacaaaactgtggctgtt

SEQ ID NO: 983 - RNA, 21 nt, synthetic construct
  misc_feature 1..21: note = artificial sequence
  source 1..21: mol_type = other RNA
gagaccagaattctcctgcaa

SEQ ID NO: 984 - RNA, 21 nt, synthetic construct
  misc_feature 1..21: note = artificial sequence
  source 1..21: mol_type = other RNA
tcgacctcaaggtcaccataa

SEQ ID NO: 985 - RNA, 21 nt, synthetic construct
  misc_feature 1..21: note = artificial sequence
  source 1..21: mol_type = other RNA
ccgcagctccaagatcaccca

SEQ ID NO: 986 - RNA, 21 nt, synthetic construct
  misc_feature 1..21: note = artificial sequence
  source 1..21: mol_type = other RNA
gagtggactatgtgtacaaga

SEQ ID NO: 987 - RNA, 21 nt, synthetic construct
  misc_feature 1..21: note = artificial sequence
  source 1..21: mol_type = other RNA
ccccatgaggttctcgtgcca

SEQ ID NO: 988 - RNA, 21 nt, synthetic construct
  misc_feature 1..21: note = artificial sequence
  source 1..21: mol_type = other RNA
gaggatgactgtctagctttc

SEQ ID NO: 989 - RNA, 21 nt, synthetic construct
  misc_feature 1..21: note = artificial sequence
  source 1..21: mol_type = other RNA
ctgcctttgtcatcttcggga

SEQ ID NO: 990 - RNA, 21 nt, synthetic construct
  misc_feature 1..21: note = artificial sequence
  source 1..21: mol_type = other RNA
aagctgtacccggttctacca

SEQ ID NO: 991 - RNA, 21 nt, synthetic construct
  misc_feature 1..21: note = artificial sequence
  source 1..21: mol_type = other RNA
ccaaagatcaactcacctgta

SEQ ID NO: 992 - RNA, 21 nt, synthetic construct
  misc_feature 1..21: note = artificial sequence
  source 1..21: mol_type = other RNA
ttgggacattccggaactcgt

SEQ ID NO: 993 - RNA, 21 nt, synthetic construct
  misc_feature 1..21: note = artificial sequence
  source 1..21: mol_type = other RNA
cagtaatgcaggacttcttca

SEQ ID NO: 994 - RNA, 21 nt, synthetic construct
  misc_feature 1..21: note = artificial sequence
  source 1..21: mol_type = other RNA
taaagcttcagttatatctca

SEQ ID NO: 995 - RNA, 21 nt, synthetic construct
  misc_feature 1..21: note = artificial sequence
  source 1..21: mol_type = other RNA
aatgtaccatgctaaggccaa

SEQ ID NO: 996 - RNA, 21 nt, synthetic construct
  misc_feature 1..21: note = artificial sequence
  source 1..21: mol_type = other RNA
caggaactgaaccttgatgtg

SEQ ID NO: 997 - RNA, 21 nt, synthetic construct
  misc_feature 1..21: note = artificial sequence
  source 1..21: mol_type = other RNA
atgaaacggagcagtgggaga

SEQ ID NO: 998 - RNA, 21 nt, synthetic construct
  misc_feature 1..21: note = artificial sequence
  source 1..21: mol_type = other RNA
gggccgtgaaggagtgcagaa

SEQ ID NO: 999 - RNA, 21 nt, synthetic construct
  misc_feature 1..21: note = artificial sequence
  source 1..21: mol_type = other RNA
tccaagtatgagctggacaaa

SEQ ID NO: 1000 - RNA, 21 nt, synthetic construct
  misc_feature 1..21: note = artificial sequence
  source 1..21: mol_type = other RNA
ttggcgtcttgcccttgtctt

SEQ ID NO: 1001 - RNA, 21 nt, synthetic construct
  misc_feature 1..21: note = artificial sequence
  source 1..21: mol_type = other RNA
ccccttcgaggtcacagtaat

SEQ ID NO: 1002 - RNA, 21 nt, synthetic construct
  misc_feature 1..21: note = artificial sequence
  source 1..21: mol_type = other RNA
ctcatcgccatcgactcccaa

SEQ ID NO: 1003 - RNA, 21 nt, synthetic construct
  misc_feature 1..21: note = artificial sequence
  source 1..21: mol_type = other RNA
atccctcaagcgcattccgat

SEQ ID NO: 1004 - RNA, 21 nt, synthetic construct
  misc_feature 1..21: note = artificial sequence
  source 1..21: mol_type = other RNA
caacagggagttcaagtcaga

SEQ ID NO: 1005 - RNA, 21 nt, synthetic construct
  misc_feature 1..21: note = artificial sequence
  source 1..21: mol_type = other RNA
tgaggacatcattgcagaaga

SEQ ID NO: 1006 - RNA, 21 nt, synthetic construct
  misc_feature 1..21: note = artificial sequence
  source 1..21: mol_type = other RNA
ctgggcgaggcgtgcaagaag

SEQ ID NO: 1007 - RNA, 21 nt, synthetic construct
  misc_feature 1..21: note = artificial sequence
  source 1..21: mol_type = other RNA
acccggttctaccatccggaa

SEQ ID NO: 1008 - RNA, 21 nt, synthetic construct
  misc_feature 1..21: note = artificial sequence
  source 1..21: mol_type = other RNA
cagtcaaggtctacgcctatt

SEQ ID NO: 1009 - RNA, 21 nt, synthetic construct
  misc_feature 1..21: note = artificial sequence
  source 1..21: mol_type = other RNA
accctggtaagcagctctaca

SEQ ID NO: 1010 - RNA, 21 nt, synthetic construct
  misc_feature 1..21: note = artificial sequence
  source 1..21: mol_type = other RNA
tcccagagagctggctgtgga

SEQ ID NO: 1011 - RNA, 21 nt, synthetic construct
  misc_feature 1..21: note = artificial sequence
  source 1..21: mol_type = other RNA
gtgctgaggagaattgcttca

SEQ ID NO: 1012 - RNA, 21 nt, synthetic construct
  misc_feature 1..21: note = artificial sequence
  source 1..21: mol_type = other RNA
tggggaagtctttgtacgtgt

SEQ ID NO: 1013 - RNA, 21 nt, synthetic construct
  misc_feature 1..21: note = artificial sequence
  source 1..21: mol_type = other RNA
tctctacgaagctcatgaata

SEQ ID NO: 1014 - RNA, 21 nt, synthetic construct
  misc_feature 1..21: note = artificial sequence
  source 1..21: mol_type = other RNA
tgggacgtggtggagaaggca

SEQ ID NO: 1015 - RNA, 21 nt, synthetic construct
  misc_feature 1..21: note = artificial sequence
  source 1..21: mol_type = other RNA
aatccgagccgttctctacaa

SEQ ID NO: 1016 - RNA, 21 nt, synthetic construct
  misc_feature 1..21: note = artificial sequence
  source 1..21: mol_type = other RNA
ctgaacaagctctgccgtgat

SEQ ID NO: 1017 - RNA, 21 nt, synthetic construct
  misc_feature 1..21: note = artificial sequence
  source 1..21: mol_type = other RNA
ctccgataggaacaccctcat

SEQ ID NO: 1018 - RNA, 21 nt, synthetic construct
  misc_feature 1..21: note = artificial sequence
  source 1..21: mol_type = other RNA
aacgtcaacttcctcctgcga

SEQ ID NO: 1019 - RNA, 21 nt, synthetic construct
  misc_feature 1..21: note = artificial sequence
  source 1..21: mol_type = other RNA
acagctgaaggaaaaggccaa

SEQ ID NO: 1020 - RNA, 21 nt, synthetic construct
  misc_feature 1..21: note = artificial sequence
  source 1..21: mol_type = other RNA
cagatccacttcaccaagaca

SEQ ID NO: 1021 - RNA, 21 nt, synthetic construct
  misc_feature 1..21: note = artificial sequence
  source 1..21: mol_type = other RNA
gccaagacgaagagaaccaga

SEQ ID NO: 1022 - RNA, 21 nt, synthetic construct
  misc_feature 1..21: note = artificial sequence
  source 1..21: mol_type = other RNA
ctaccatcatttcatcagtga

SEQ ID NO: 1023 - RNA, 21 nt, synthetic construct
  misc_feature 1..21: note = artificial sequence
  source 1..21: mol_type = other RNA
tcgaaacgagcaggtggaaat

SEQ ID NO: 1024 - RNA, 21 nt, synthetic construct
  misc_feature 1..21: note = artificial sequence
  source 1..21: mol_type = other RNA
gagatactacggtggtggcta

SEQ ID NO: 1025 - RNA, 21 nt, synthetic construct
  misc_feature 1..21: note = artificial sequence
  source 1..21: mol_type = other RNA
ccctcatcatctacctggaca

SEQ ID NO: 1026 - RNA, 21 nt, synthetic construct
  misc_feature 1..21: note = artificial sequence
  source 1..21: mol_type = other RNA
ctccatcctacttgaaacctc

SEQ ID NO: 1027 - RNA, 21 nt, synthetic construct
  misc_feature 1..21: note = artificial sequence
  source 1..21: mol_type = other RNA
acgtcaacttcctcctgcgaa

SEQ ID NO: 1028 - RNA, 21 nt, synthetic construct
  misc_feature 1..21: note = artificial sequence
  source 1..21: mol_type = other RNA
ctgcggctaccctactctgtt

SEQ ID NO: 1029 - RNA, 21 nt, synthetic construct
  misc_feature 1..21: note = artificial sequence
  source 1..21: mol_type = other RNA
accgagagcatggttgtcttt

SEQ ID NO: 1030 - RNA, 21 nt, synthetic construct
  misc_feature 1..21: note = artificial sequence
  source 1..21: mol_type = other RNA
ccgggtggtactggtggccgt

SEQ ID NO: 1031 - RNA, 21 nt, synthetic construct
  misc_feature 1..21: note = artificial sequence
  source 1..21: mol_type = other RNA
aagacctggtggggaagtctt

SEQ ID NO: 1032 - RNA, 21 nt, synthetic construct
  misc_feature 1..21: note = artificial sequence
  source 1..21: mol_type = other RNA
cacggtgcgcacgaagaagca

SEQ ID NO: 1033 - RNA, 21 nt, synthetic construct
  misc_feature 1..21: note = artificial sequence
  source 1..21: mol_type = other RNA
tgcgcccgtgatacaccaaga

SEQ ID NO: 1034 - RNA, 21 nt, synthetic construct
  misc_feature 1..21: note = artificial sequence
  source 1..21: mol_type = other RNA
cggcgccttcaccgagagcat

SEQ ID NO: 1035 - RNA, 21 nt, synthetic construct
  misc_feature 1..21: note = artificial sequence
  source 1..21: mol_type = other RNA
gttcaccaatactttaatgta

SEQ ID NO: 1036 - RNA, 21 nt, synthetic construct
  misc_feature 1..21: note = artificial sequence
  source 1..21: mol_type = other RNA
tcaaagttcaccaatacttta

SEQ ID NO: 1037 - RNA, 21 nt, synthetic construct
  misc_feature 1..21: note = artificial sequence
  source 1..21: mol_type = other RNA
agcagaagacctggtggggaa

SEQ ID NO: 1038 - RNA, 21 nt, synthetic construct
  misc_feature 1..21: note = artificial sequence
  source 1..21: mol_type = other RNA
tgcctttgtcatcttcgggat

SEQ ID NO: 1039 - RNA, 21 nt, synthetic construct
  misc_feature 1..21: note = artificial sequence
  source 1..21: mol_type = other RNA
ctgtccacgacttcccaggca

SEQ ID NO: 1040 - RNA, 21 nt, synthetic construct
  misc_feature 1..21: note = artificial sequence
  source 1..21: mol_type = other RNA
cagccccatcaagtgcagaga

SEQ ID NO: 1041 - RNA, 21 nt, synthetic construct
  misc_feature 1..21: note = artificial sequence
  source 1..21: mol_type = other RNA
cttcatggtgttccaagcctt

SEQ ID NO: 1042 - RNA, 21 nt, synthetic construct
  misc_feature 1..21: note = artificial sequence
  source 1..21: mol_type = other RNA
cctgcgatcagaagagaccaa

SEQ ID NO: 1043 - RNA, 21 nt, synthetic construct
  misc_feature 1..21: note = artificial sequence
  source 1..21: mol_type = other RNA
gtcggtggtgacaatgtacca

SEQ ID NO: 1044 - RNA, 21 nt, synthetic construct
  misc_feature 1..21: note = artificial sequence
  source 1..21: mol_type = other RNA
tcccgtcgtgcgttggctcaa

SEQ ID NO: 1045 - RNA, 21 nt, synthetic construct
  misc_feature 1..21: note = artificial sequence
  source 1..21: mol_type = other RNA
cccgtcgtgcgttggctcaat

SEQ ID NO: 1046 - RNA, 21 nt, synthetic construct
  misc_feature 1..21: note = artificial sequence
  source 1..21: mol_type = other RNA
tcacagtcacagctgaaggaa

SEQ ID NO: 1047 - RNA, 21 nt, synthetic construct
  misc_feature 1..21: note = artificial sequence
  source 1..21: mol_type = other RNA
caagtacttcaaaccaggaat

SEQ ID NO: 1048 - RNA, 21 nt, synthetic construct
  misc_feature 1..21: note = artificial sequence
  source 1..21: mol_type = other RNA
atgaacagagatactacggtg

SEQ ID NO: 1049 - RNA, 21 nt, synthetic construct
  misc_feature 1..21: note = artificial sequence
  source 1..21: mol_type = other RNA
gaccaaggaaaatgagggttt

SEQ ID NO: 1050 - RNA, 21 nt, synthetic construct
  misc_feature 1..21: note = artificial sequence
  source 1..21: mol_type = other RNA
acccaccgtatccactgggaa

SEQ ID NO: 1051 - RNA, 21 nt, synthetic construct
  misc_feature 1..21: note = artificial sequence
  source 1..21: mol_type = other RNA
cagctccatcctacttgaaac

SEQ ID NO: 1052 - RNA, 21 nt, synthetic construct
  misc_feature 1..21: note = artificial sequence
  source 1..21: mol_type = other RNA
ctgcgaggacggcatgcggga

SEQ ID NO: 1053 - RNA, 21 nt, synthetic construct
  misc_feature 1..21: note = artificial sequence
  source 1..21: mol_type = other RNA
tgccaagagggagaggctgaa

SEQ ID NO: 1054 - RNA, 21 nt, synthetic construct
  misc_feature 1..21: note = artificial sequence
  source 1..21: mol_type = other RNA
ctcaggcagtgacatggtgca

SEQ ID NO: 1055 - RNA, 21 nt, synthetic construct
  misc_feature 1..21: note = artificial sequence
  source 1..21: mol_type = other RNA
cacccaccgtatccactggga

SEQ ID NO: 1056 - RNA, 21 nt, synthetic construct
  misc_feature 1..21: note = artificial sequence
  source 1..21: mol_type = other RNA
cacagctgaaggaaaaggcca

SEQ ID NO: 1057 - RNA, 21 nt, synthetic construct
  misc_feature 1..21: note = artificial sequence
  source 1..21: mol_type = other RNA
cgcctattacaacctggagga

SEQ ID NO: 1058 - RNA, 21 nt, synthetic construct
  misc_feature 1..21: note = artificial sequence
  source 1..21: mol_type = other RNA
ctactgcagctaaaagacttt

SEQ ID NO: 1059 - RNA, 21 nt, synthetic construct
  misc_feature 1..21: note = artificial sequence
  source 1..21: mol_type = other RNA
cgaggccaagatccgctacta

SEQ ID NO: 1060 - RNA, 21 nt, synthetic construct
  misc_feature 1..21: note = artificial sequence
  source 1..21: mol_type = other RNA
tgaggagaattgcttcataca

SEQ ID NO: 1061 - RNA, 21 nt, synthetic construct
  misc_feature 1..21: note = artificial sequence
  source 1..21: mol_type = other RNA
agccccatcaagtgcagagaa

SEQ ID NO: 1062 - RNA, 21 nt, synthetic construct
  misc_feature 1..21: note = artificial sequence
  source 1..21: mol_type = other RNA
caagtgcagagaagccctgaa

SEQ ID NO: 1063 - RNA, 21 nt, synthetic construct
  misc_feature 1..21: note = artificial sequence
  source 1..21: mol_type = other RNA
aagccctgaagctggaggaga

SEQ ID NO: 1064 - RNA, 21 nt, synthetic construct
  misc_feature 1..21: note = artificial sequence
  source 1..21: mol_type = other RNA
ctccttgtcttctcagaacca

SEQ ID NO: 1065 - RNA, 21 nt, synthetic construct
  misc_feature 1..21: note = artificial sequence
  source 1..21: mol_type = other RNA
cagacccggggagaccctcaa

SEQ ID NO: 1066 - RNA, 21 nt, synthetic construct
  misc_feature 1..21: note = artificial sequence
  source 1..21: mol_type = other RNA
ttgtcttgggacattccggaa

SEQ ID NO: 1067 - RNA, 21 nt, synthetic construct
  misc_feature 1..21: note = artificial sequence
  source 1..21: mol_type = other RNA
cggggagaccaggatgccact

SEQ ID NO: 1068 - RNA, 21 nt, synthetic construct
  misc_feature 1..21: note = artificial sequence
  source 1..21: mol_type = other RNA
tgcatctctcagtgctacgta

SEQ ID NO: 1069 - RNA, 21 nt, synthetic construct
  misc_feature 1..21: note = artificial sequence
  source 1..21: mol_type = other RNA
ttacggaacaacaacgagaaa

SEQ ID NO: 1070 - RNA, 21 nt, synthetic construct
  misc_feature 1..21: note = artificial sequence
  source 1..21: mol_type = other RNA
cgggaaggacacttgggtgga

SEQ ID NO: 1071 - RNA, 21 nt, synthetic construct
  misc_feature 1..21: note = artificial sequence
  source 1..21: mol_type = other RNA
tgcaggttggacagcagcgca

SEQ ID NO: 1072 - RNA, 21 nt, synthetic construct
  misc_feature 1..21: note = artificial sequence
  source 1..21: mol_type = other RNA
tacaattaccggcagaaccaa

SEQ ID NO: 1073 - RNA, 21 nt, synthetic construct
  misc_feature 1..21: note = artificial sequence
  source 1..21: mol_type = other RNA
gacccgactggtcaaggttca

SEQ ID NO: 1074 - RNA, 21 nt, synthetic construct
  misc_feature 1..21: note = artificial sequence
  source 1..21: mol_type = other RNA
ggccttggagctcatcaagaa

SEQ ID NO: 1075 - RNA, 21 nt, synthetic construct
  misc_feature 1..21: note = artificial sequence
  source 1..21: mol_type = other RNA
aagacccgactggtcaaggtt

SEQ ID NO: 1076 - RNA, 21 nt, synthetic construct
  misc_feature 1..21: note = artificial sequence
  source 1..21: mol_type = other RNA
cggggcccgggtggtactggt

SEQ ID NO: 1077 - RNA, 21 nt, synthetic construct
  misc_feature 1..21: note = artificial sequence
  source 1..21: mol_type = other RNA
atgactttgacgagtacatca

SEQ ID NO: 1078 - RNA, 21 nt, synthetic construct
  misc_feature 1..21: note = artificial sequence
  source 1..21: mol_type = other RNA
aggcgtcaccagcagaccgta

SEQ ID NO: 1079 - RNA, 21 nt, synthetic construct
  misc_feature 1..21: note = artificial sequence
  source 1..21: mol_type = other RNA
ctgcctttgcggccttcgtga

SEQ ID NO: 1080 - RNA, 21 nt, synthetic construct
  misc_feature 1..21: note = artificial sequence
  source 1..21: mol_type = other RNA
tgaggatgactgtctagcttt

SEQ ID NO: 1081 - RNA, 21 nt, synthetic construct
  misc_feature 1..21: note = artificial sequence
  source 1..21: mol_type = other RNA
tgggcagcagatgaccctgaa

SEQ ID NO: 1082 - RNA, 21 nt, synthetic construct
  misc_feature 1..21: note = artificial sequence
  source 1..21: mol_type = other RNA
cctggaagaacggctggacaa

SEQ ID NO: 1083 - RNA, 21 nt, synthetic construct
  misc_feature 1..21: note = artificial sequence
  source 1..21: mol_type = other RNA
tcggcatgacgcccacggtca

SEQ ID NO: 1084 - RNA, 21 nt, synthetic construct
  misc_feature 1..21: note = artificial sequence
  source 1..21: mol_type = other RNA
gcgtgttcgtgctgaataaga

SEQ ID NO: 1085 - RNA, 21 nt, synthetic construct
  misc_feature 1..21: note = artificial sequence
  source 1..21: mol_type = other RNA
tcgtgctgaataagaagaaca

SEQ ID NO: 1086 - RNA, 21 nt, synthetic construct
  misc_feature 1..21: note = artificial sequence
  source 1..21: mol_type = other RNA
tccctgaaggtcgtgccggaa

SEQ ID NO: 1087 - RNA, 21 nt, synthetic construct
  misc_feature 1..21: note = artificial sequence
  source 1..21: mol_type = other RNA
ttgaaagagccaccgaaaaat

SEQ ID NO: 1088 - RNA, 21 nt, synthetic construct
  misc_feature 1..21: note = artificial sequence
  source 1..21: mol_type = other RNA
tacgggaagaaagtggaggga

SEQ ID NO: 1089 - RNA, 21 nt, synthetic construct
  misc_feature 1..21: note = artificial sequence
  source 1..21: mol_type = other RNA
gcctggagcagtcaaggtcta

SEQ ID NO: 1090 - RNA, 21 nt, synthetic construct
  misc_feature 1..21: note = artificial sequence
  source 1..21: mol_type = other RNA
gctgtggaacgttgaggactt

SEQ ID NO: 1091 - RNA, 21 nt, synthetic construct
  misc_feature 1..21: note = artificial sequence
  source 1..21: mol_type = other RNA
aaggtcaccataaaaccagca

SEQ ID NO: 1092 - RNA, 21 nt, synthetic construct
  misc_feature 1..21: note = artificial sequence
  source 1..21: mol_type = other RNA
gggcctcttcttaacaaattt

SEQ ID NO: 1093 - RNA, 21 nt, synthetic construct
  misc_feature 1..21: note = artificial sequence
  source 1..21: mol_type = other RNA
ccaggatggcgaacagaggat

SEQ ID NO: 1094 - RNA, 21 nt, synthetic construct
  misc_feature 1..21: note = artificial sequence
  source 1..21: mol_type = other RNA
ctgggccgtgaaggagtgcag

SEQ ID NO: 1095 - RNA, 21 nt, synthetic construct
  misc_feature 1..21: note = artificial sequence
  source 1..21: mol_type = other RNA
gaggacatcattgcagaagag

SEQ ID NO: 1096 - RNA, 21 nt, synthetic construct
  misc_feature 1..21: note = artificial sequence
  source 1..21: mol_type = other RNA
gatgaggacatcattgcagaa

SEQ ID NO: 1097 - RNA, 21 nt, synthetic construct
  misc_feature 1..21: note = artificial sequence
  source 1..21: mol_type = other RNA
atggcgtggccaaactcagca

SEQ ID NO: 1098 - RNA, 21 nt, synthetic construct
  misc_feature 1..21: note = artificial sequence
  source 1..21: mol_type = other RNA
aacgtggaggccacatcctat

SEQ ID NO: 1099 - RNA, 21 nt, synthetic construct
  misc_feature 1..21: note = artificial sequence
  source 1..21: mol_type = other RNA
atctctacgaagctcatgaat

SEQ ID NO: 1100 - RNA, 21 nt, synthetic construct
  misc_feature 1..21: note = artificial sequence
  source 1..21: mol_type = other RNA
acagcaccgtgggcaactcca

SEQ ID NO: 1101 - RNA, 21 nt, synthetic construct
  misc_feature 1..21: note = artificial sequence
  source 1..21: mol_type = other RNA
cctgccaggcagcatcactaa

SEQ ID NO: 1102 - RNA, 21 nt, synthetic construct
  misc_feature 1..21: note = artificial sequence
  source 1..21: mol_type = other RNA
gtactctatcatcacccccaa

SEQ ID NO: 1103 - RNA, 21 nt, synthetic construct
  misc_feature 1..21: note = artificial sequence
  source 1..21: mol_type = other RNA
ccgacgcagggctgaccttca

SEQ ID NO: 1104 - RNA, 21 nt, synthetic construct
  misc_feature 1..21: note = artificial sequence
  source 1..21: mol_type = other RNA
aaagcaggagacttccttgaa

SEQ ID NO: 1105 - RNA, 21 nt, synthetic construct
  misc_feature 1..21: note = artificial sequence
  source 1..21: mol_type = other RNA
aaagttcaccaatactttaat

SEQ ID NO: 1106 - RNA, 21 nt, synthetic construct
  misc_feature 1..21: note = artificial sequence
  source 1..21: mol_type = other RNA
ctgcccagtttcgaggtcata

SEQ ID NO: 1107 - RNA, 21 nt, synthetic construct
  misc_feature 1..21: note = artificial sequence
  source 1..21: mol_type = other RNA
caccaatactttaatgtagag

SEQ ID NO: 1108 - RNA, 21 nt, synthetic construct
  misc_feature 1..21: note = artificial sequence
  source 1..21: mol_type = other RNA
atactttaatgtagagcttat

SEQ ID NO: 1109 - RNA, 21 nt, synthetic construct
  misc_feature 1..21: note = artificial sequence
  source 1..21: mol_type = other RNA
gcctacagagaaattctacta

SEQ ID NO: 1110 - RNA, 21 nt, synthetic construct
  misc_feature 1..21: note = artificial sequence
  source 1..21: mol_type = other RNA
gagaaattctactacatctat

SEQ ID NO: 1111 - RNA, 21 nt, synthetic construct
  misc_feature 1..21: note = artificial sequence
  source 1..21: mol_type = other RNA
ctacgaagctcatgaatatat

SEQ ID NO: 1112 - RNA, 21 nt, synthetic construct
  misc_feature 1..21: note = artificial sequence
  source 1..21: mol_type = other RNA
ctactacatctataacgagaa

SEQ ID NO: 1113 - RNA, 21 nt, synthetic construct
  misc_feature 1..21: note = artificial sequence
  source 1..21: mol_type = other RNA
ctgaggagaattgcttcatac

SEQ ID NO: 1114 - RNA, 21 nt, synthetic construct
  misc_feature 1..21: note = artificial sequence
  source 1..21: mol_type = other RNA
gagcagtcaaggtctacgcct

SEQ ID NO: 1115 - RNA, 21 nt, synthetic construct
  misc_feature 1..21: note = artificial sequence
  source 1..21: mol_type = other RNA
caccgcattttctcctggcgt

SEQ ID NO: 1116 - RNA, 21 nt, synthetic construct
  misc_feature 1..21: note = artificial sequence
  source 1..21: mol_type = other RNA
ctggtggcgtactacacgctg

SEQ ID NO: 1117 - RNA, 21 nt, synthetic construct
  misc_feature 1..21: note = artificial sequence
  source 1..21: mol_type = other RNA
tgccgctgtgctgaggagaat

SEQ ID NO: 1118 - RNA, 21 nt, synthetic construct
  misc_feature 1..21: note = artificial sequence
  source 1..21: mol_type = other RNA
acgcagggctgaccttcacga

SEQ ID NO: 1119 - RNA, 21 nt, synthetic construct
  misc_feature 1..21: note = artificial sequence
  source 1..21: mol_type = other RNA
atgccaagacgaagagaacca

SEQ ID NO: 1120 - RNA, 21 nt, synthetic construct
  misc_feature 1..21: note = artificial sequence
  source 1..21: mol_type = other RNA
catcacggtgcgcacgaagaa

SEQ ID NO: 1121 - RNA, 21 nt, synthetic construct
  misc_feature 1..21: note = artificial sequence
  source 1..21: mol_type = other RNA
gagcatggttgtctttgggtg

SEQ ID NO: 1122 - RNA, 21 nt, synthetic construct
  misc_feature 1..21: note = artificial sequence
  source 1..21: mol_type = other RNA
gacctcaaggtcaccataaaa

SEQ ID NO: 1123 - RNA, 21 nt, synthetic construct
  misc_feature 1..21: note = artificial sequence
  source 1..21: mol_type = other RNA
aggacactgtgcagtctctaa

SEQ ID NO: 1124 - RNA, 21 nt, synthetic construct
  misc_feature 1..21: note = artificial sequence
  source 1..21: mol_type = other RNA
cagccagaagcccttgagcat

SEQ ID NO: 1125 - RNA, 21 nt, synthetic construct
  misc_feature 1..21: note = artificial sequence
  source 1..21: mol_type = other RNA
cactgtgcagtctctaaccca

SEQ ID NO: 1126 - RNA, 21 nt, synthetic construct
  misc_feature 1..21: note = artificial sequence
  source 1..21: mol_type = other RNA
cacctgtaataaattcgacct

SEQ ID NO: 1127 - RNA, 21 nt, synthetic construct
  misc_feature 1..21: note = artificial sequence
  source 1..21: mol_type = other RNA
gccctacagcaccgtgggcaa

SEQ ID NO: 1128 - RNA, 21 nt, synthetic construct
  misc_feature 1..21: note = artificial sequence
  source 1..21: mol_type = other RNA
accgtgggcaactccaacaat

SEQ ID NO: 1129 - RNA, 21 nt, synthetic construct
  misc_feature 1..21: note = artificial sequence
  source 1..21: mol_type = other RNA
cgagtacatcatggccattga

SEQ ID NO: 1130 - RNA, 21 nt, synthetic construct
  misc_feature 1..21: note = artificial sequence
  source 1..21: mol_type = other RNA
tgacagatacatctccaagta

SEQ ID NO: 1131 - RNA, 21 nt, synthetic construct
  misc_feature 1..21: note = artificial sequence
  source 1..21: mol_type = other RNA
gtgttgacagatacatctcca

SEQ ID NO: 1132 - RNA, 21 nt, synthetic construct
  misc_feature 1..21: note = artificial sequence
  source 1..21: mol_type = other RNA
atggtgttgacagatacatct

SEQ ID NO: 1133 - RNA, 21 nt, synthetic construct
  misc_feature 1..21: note = artificial sequence
  source 1..21: mol_type = other RNA
accatcaagtcaggctcggat

SEQ ID NO: 1134 - RNA, 21 nt, synthetic construct
  misc_feature 1..21: note = artificial sequence
  source 1..21: mol_type = other RNA
atgatgactggctttgctcca

SEQ ID NO: 1135 - RNA, 21 nt, synthetic construct
  misc_feature 1..21: note = artificial sequence
  source 1..21: mol_type = other RNA
cgtgggcaactccaacaatta

SEQ ID NO: 1136 - RNA, 21 nt, synthetic construct
  misc_feature 1..21: note = artificial sequence
  source 1..21: mol_type = other RNA
taagatctgggacgtggtgga

SEQ ID NO: 1137 - RNA, 21 nt, synthetic construct
  misc_feature 1..21: note = artificial sequence
  source 1..21: mol_type = other RNA
gatctgggacgtggtggagaa

SEQ ID NO: 1138 - RNA, 21 nt, synthetic construct
  misc_feature 1..21: note = artificial sequence
  source 1..21: mol_type = other RNA
cagagaagccctgaagctgga

SEQ ID NO: 1139 - RNA, 21 nt, synthetic construct
  misc_feature 1..21: note = artificial sequence
  source 1..21: mol_type = other RNA
ggggagaccaggatgccacta

SEQ ID NO: 1140 - RNA, 21 nt, synthetic construct
  misc_feature 1..21: note = artificial sequence
  source 1..21: mol_type = other RNA
ctgcaccccgggcagtgggaa

SEQ ID NO: 1141 - RNA, 21 nt, synthetic construct
  misc_feature 1..21: note = artificial sequence
  source 1..21: mol_type = other RNA
agagaagcccaacctcagcta

SEQ ID NO: 1142 - RNA, 21 nt, synthetic construct
  misc_feature 1..21: note = artificial sequence
  source 1..21: mol_type = other RNA
aacaattacctgcatctctca

SEQ ID NO: 1143 - RNA, 21 nt, synthetic construct
  misc_feature 1..21: note = artificial sequence
  source 1..21: mol_type = other RNA
aggagtgcagaaagaggacat

SEQ ID NO: 1144 - RNA, 21 nt, synthetic construct
  misc_feature 1..21: note = artificial sequence
  source 1..21: mol_type = other RNA
aactagtgctgtccagtgaga

SEQ ID NO: 1145 - RNA, 21 nt, synthetic construct
  misc_feature 1..21: note = artificial sequence
  source 1..21: mol_type = other RNA
aagtcctcgttgtccgttcca

SEQ ID NO: 1146 - RNA, 21 nt, synthetic construct
  misc_feature 1..21: note = artificial sequence
  source 1..21: mol_type = other RNA
ctcgggggaggttgtgctgag

SEQ ID NO: 1147 - RNA, 21 nt, synthetic construct
  misc_feature 1..21: note = artificial sequence
  source 1..21: mol_type = other RNA
ggacatcattgcagaagagaa

SEQ ID NO: 1148 - RNA, 21 nt, synthetic construct
  misc_feature 1..21: note = artificial sequence
  source 1..21: mol_type = other RNA
tccacgacttcccaggcaaaa

SEQ ID NO: 1149 - RNA, 21 nt, synthetic construct
  misc_feature 1..21: note = artificial sequence
  source 1..21: mol_type = other RNA
tggattacggaacaacaacga

SEQ ID NO: 1150 - RNA, 21 nt, synthetic construct
  misc_feature 1..21: note = artificial sequence
  source 1..21: mol_type = other RNA
cctggtggggaagtctttgta

SEQ ID NO: 1151 - RNA, 21 nt, synthetic construct
  misc_feature 1..21: note = artificial sequence
  source 1..21: mol_type = other RNA
aggcctggagtttattcggaa

SEQ ID NO: 1152 - RNA, 23 nt, synthetic construct
  misc_feature 1..23: note = artificial sequence
  source 1..23: mol_type = other RNA
tttccggacctcaaataagcctt

SEQ ID NO: 1153 - RNA, 21 nt, synthetic construct
  misc_feature 1..21: note = artificial sequence
  source 1..21: mol_type = other RNA
aggcctggagtttattcggaa

SEQ ID NO: 1154 - RNA, 23 nt, synthetic construct
  misc_feature 1..23: note = artificial sequence
  source 1..23: mol_type = other RNA
tttccggacctcaaataagcctt

SEQ ID NO: 1155 - DNA, 19 bp, synthetic construct
  misc_feature 1..19: note = single stranded DNA
  source 1..19: mol_type = other DNA
gcgaagcccctactccact

SEQ ID NO: 1156 - RNA, 19 nt, synthetic construct
  misc_feature 1..19: note = inhibitory RNA
  source 1..19: mol_type = other RNA
tgagagaagaccttgacca

SEQ ID NO: 1157 - RNA, 19 nt, synthetic construct
  misc_feature 1..19: note = inhibitory RNA
  source 1..19: mol_type = other RNA
ttgtagtagcggatcttgg

SEQ ID NO: 1158 - RNA, 19 nt, synthetic construct
  misc_feature 1..19: note = inhibitory RNA
  source 1..19: mol_type = other RNA
tgagagacgaccttgacca

SEQ ID NO: 1159 - RNA, 19 nt, synthetic construct
  misc_feature 1..19: note = inhibitory RNA
  source 1..19: mol_type = other RNA
tgagagaataccttgacca

SEQ ID NO: 1160 - RNA, 19 nt, synthetic construct
  misc_feature 1..19: note = inhibitory RNA
  source 1..19: mol_type = other RNA
tgagagaagaccttgaccg

SEQ ID NO: 1161 - RNA, 19 nt, synthetic construct
  misc_feature 1..19: note = inhibitory RNA
  source 1..19: mol_type = other RNA
atgagcttcgtagagattc

SEQ ID NO: 1162 - RNA, 19 nt, synthetic construct
  misc_feature 1..19: note = inhibitory RNA
  source 1..19: mol_type = other RNA
ttctgtctggatgaagagg

SEQ ID NO: 1163 - RNA, 19 nt, synthetic construct
  misc_feature 1..19: note = inhibitory RNA
  source 1..19: mol_type = other RNA
tgagagacgaccttgaccg

SEQ ID NO: 1164 - RNA, 19 nt, synthetic construct
  misc_feature 1..19: note = inhibitory RNA
  source 1..19: mol_type = other RNA
tatatattcatgagcttcg

SEQ ID NO: 1165 - RNA, 21 nt, synthetic construct
  misc_feature 1..21: note = inhibitory RNA
  source 1..21: mol_type = other RNA
gagccgttctctacaattact

SEQ ID NO: 1166 - RNA, 23 nt, synthetic construct
  misc_feature 1..23: note = inhibitory RNA
  source 1..23: mol_type = other RNA
agtaattgtagagaacggctcgg

SEQ ID NO: 1167 - RNA, 19 nt, synthetic construct
  misc_feature 1..19: note = inhibitory RNA
  source 1..19: mol_type = other RNA
tgaagagaaatccattgtc

SEQ ID NO: 1168 - RNA, 19 nt, synthetic construct
  misc_feature 1..19: note = inhibitory RNA
  source 1..19: mol_type = other RNA
tatagttgtaaacagttcc

SEQ ID NO: 1169 - RNA, 19 nt, synthetic construct
  misc_feature 1..19: note = inhibitory RNA
  source 1..19: mol_type = other RNA
gtacaacagaatatggtat

SEQ ID NO: 1170 - RNA, 19 nt, synthetic construct
  misc_feature 1..19: note = inhibitory RNA
  source 1..19: mol_type = other RNA
caacattgtgttttgcatt

SEQ ID NO: 1171 - RNA, 19 nt, synthetic construct
  misc_feature 1..19: note = inhibitory RNA
  source 1..19: mol_type = other RNA
gagaaatccattgtcatag

SEQ ID NO: 1172 - RNA, 19 nt, synthetic construct
  misc_feature 1..19: note = inhibitory RNA
  source 1..19: mol_type = other RNA
agtaatttcaaaattctta

SEQ ID NO: 1173 - RNA, 19 nt, synthetic construct
  misc_feature 1..19: note = inhibitory RNA
  source 1..19: mol_type = other RNA
agtaattatagtgagttat

SEQ ID NO: 1174 - RNA, 19 nt, synthetic construct
  misc_feature 1..19: note = inhibitory RNA
  source 1..19: mol_type = other RNA
gaatgtttatactttgata

SEQ ID NO: 1175 - RNA, 19 nt, synthetic construct
  misc_feature 1..19: note = inhibitory RNA
  source 1..19: mol_type = other RNA
catattctttaacttcaaa

SEQ ID NO: 1176 - RNA, 19 nt, synthetic construct
  misc_feature 1..19: note = inhibitory RNA
  source 1..19: mol_type = other RNA
cattgtcataggttattgg

SEQ ID NO: 1177 - RNA, 19 nt, synthetic construct
  misc_feature 1..19: note = inhibitory RNA
  source 1..19: mol_type = other RNA
tttaagtcttctcttattc

SEQ ID NO: 1178 - RNA, 19 nt, synthetic construct
  misc_feature 1..19: note = inhibitory RNA
  source 1..19: mol_type = other RNA
atataaaggtacttgttgt

SEQ ID NO: 1179 - RNA, 19 nt, synthetic construct
  misc_feature 1..19: note = inhibitory RNA
  source 1..19: mol_type = other RNA
aattatagtgagttatttt

SEQ ID NO: 1180 - RNA, 19 nt, synthetic construct
  misc_feature 1..19: note = inhibitory RNA
  source 1..19: mol_type = other RNA
tgtaaacagttcctttcaa

SEQ ID NO: 1181 - RNA, 19 nt, synthetic construct
  misc_feature 1..19: note = inhibitory RNA
  source 1..19: mol_type = other RNA
tacagcaatataaaggtac

SEQ ID NO: 1182 - RNA, 19 nt, synthetic construct
  misc_feature 1..19: note = inhibitory RNA
  source 1..19: mol_type = other RNA
aagtcttctcttattccaa

SEQ ID NO: 1183 - RNA, 19 nt, synthetic construct
  misc_feature 1..19: note = inhibitory RNA
  source 1..19: mol_type = other RNA
ggataaatgaacatggcct

SEQ ID NO: 1184 - RNA, 19 nt, synthetic construct
  misc_feature 1..19: note = inhibitory RNA
  source 1..19: mol_type = other RNA
caaggttcatcattttctt

SEQ ID NO: 1185 - RNA, 19 nt, synthetic construct
  misc_feature 1..19: note = inhibitory RNA
  source 1..19: mol_type = other RNA
gataattccaatatgatca

SEQ ID NO: 1186 - RNA, 19 nt, synthetic construct
  misc_feature 1..19: note = inhibitory RNA
  source 1..19: mol_type = other RNA
aaatccattgtcataggtt

SEQ ID NO: 1187 - RNA, 19 nt, synthetic construct
  misc_feature 1..19: note = inhibitory RNA
  source 1..19: mol_type = other RNA
tcaggaaaagagataattc

SEQ ID NO: 1188 - RNA, 19 nt, synthetic construct
  misc_feature 1..19: note = inhibitory RNA
  source 1..19: mol_type = other RNA
agttattttgtcaatatat

SEQ ID NO: 1189 - RNA, 19 nt, synthetic construct
  misc_feature 1..19: note = inhibitory RNA
  source 1..19: mol_type = other RNA
agacatctttgaacacctt

SEQ ID NO: 1190 - RNA, 19 nt, synthetic construct
  misc_feature 1..19: note = inhibitory RNA
  source 1..19: mol_type = other RNA
tataagcatatgcaatctc

SEQ ID NO: 1191 - RNA, 19 nt, synthetic construct
  misc_feature 1..19: note = inhibitory RNA
  source 1..19: mol_type = other RNA
aagacatattctttaactt

SEQ ID NO: 1192 - RNA, 23 nt, synthetic construct
  misc_feature 1..23: note = inhibitory RNA
  source 1..23: mol_type = other RNA
acaaaagcaaaacaggtctagaa

SEQ ID NO: 1193 - RNA, 21 nt, synthetic construct
  misc_feature 1..21: note = inhibitory RNA
  source 1..21: mol_type = other RNA
caggtctggaatgcaaagtca

SEQ ID NO: 1194 - RNA, 21 nt, synthetic construct
  misc_feature 1..21: note = inhibitory RNA
  source 1..21: mol_type = other RNA
tgactttgcattccagacctg

SEQ ID NO: 1195 - RNA, 19 nt, synthetic construct
  misc_feature 1..19: note = inhibitory RNA
  source 1..19: mol_type = other RNA
agacataattccagaagaa

SEQ ID NO: 1196 - RNA, 21 nt, synthetic construct
  misc_feature 1..21: note = inhibitory RNA
  source 1..21: mol_type = other RNA
ttcttctggaattatgtcttt

SEQ ID NO: 1197 - RNA, 19 nt, synthetic construct
  misc_feature 1..19: note = inhibitory RNA
  source 1..19: mol_type = other RNA
gggaaacggtggtggagaa

SEQ ID NO: 1198 - RNA, 21 nt, synthetic construct
  misc_feature 1..21: note = inhibitory RNA
  source 1..21: mol_type = other RNA
ttctccaccaccgtttccctt

SEQ ID NO: 1199 - RNA, 19 nt, synthetic construct
  misc_feature 1..19: note = inhibitory RNA
  source 1..19: mol_type = other RNA
ccgcagagtttgaggtgaa

SEQ ID NO: 1200 - RNA, 21 nt, synthetic construct
  misc_feature 1..21: note = inhibitory RNA
  source 1..21: mol_type = other RNA
ttcacctcaaactctgcggtt

SEQ ID NO: 1201 - RNA, 19 nt, synthetic construct
  misc_feature 1..19: note = inhibitory RNA
  source 1..19: mol_type = other RNA
gttgaaagaaccagagaaa

SEQ ID NO: 1202 - RNA, 21 nt, synthetic construct
  misc_feature 1..21: note = inhibitory RNA
  source 1..21: mol_type = other RNA
tttctctggttctttcaactt

SEQ ID NO: 1203 - RNA, 19 nt, synthetic construct
  misc_feature 1..19: note = inhibitory RNA
  source 1..19: mol_type = other RNA
agtttgaggtgaaggaata

SEQ ID NO: 1204 - RNA, 21 nt, synthetic construct
  misc_feature 1..21: note = inhibitory RNA
  source 1..21: mol_type = other RNA
tattccttcacctcaaacttt

SEQ ID NO: 1205 - RNA, 19 nt, synthetic construct
  misc_feature 1..19: note = inhibitory RNA
  source 1..19: mol_type = other RNA
gggtggaactgttgcataa

SEQ ID NO: 1206 - RNA, 21 nt, synthetic construct
  misc_feature 1..21: note = inhibitory RNA
  source 1..21: mol_type = other RNA
ttatgcaacagttccaccctt

SEQ ID NO: 1207 - RNA, 19 nt, synthetic construct
  misc_feature 1..19: note = inhibitory RNA
  source 1..19: mol_type = other RNA
ggacaaagctggtcagtac

SEQ ID NO: 1208 - RNA, 21 nt, synthetic construct
  misc_feature 1..21: note = inhibitory RNA
  source 1..21: mol_type = other RNA
gtactgaccagctttgtcctt

SEQ ID NO: 1209 - RNA, 19 nt, synthetic construct
  misc_feature 1..19: note = inhibitory RNA
  source 1..19: mol_type = other RNA
ggagtgaattggaggaaga

SEQ ID NO: 1210 - RNA, 21 nt, synthetic construct
  misc_feature 1..21: note = inhibitory RNA
  source 1..21: mol_type = other RNA
tcttcctccaattcactcctt

SEQ ID NO: 1211 - RNA, 19 nt, synthetic construct
  misc_feature 1..19: note = inhibitory RNA
  source 1..19: mol_type = other RNA
ccatagaagagttgaaaga

SEQ ID NO: 1212 - RNA, 21 nt, synthetic construct
  misc_feature 1..21: note = inhibitory RNA
  source 1..21: mol_type = other RNA
tctttcaactcttctatggtt

SEQ ID NO: 1213 - RNA, 19 nt, synthetic construct
  misc_feature 1..19: note = inhibitory RNA
  source 1..19: mol_type = other RNA
acatagagctgttggatga

SEQ ID NO: 1214 - RNA, 21 nt, synthetic construct
  misc_feature 1..21: note = inhibitory RNA
  source 1..21: mol_type = other RNA
tcatccaacagctctatgttt

SEQ ID NO: 1215 - RNA, 19 nt, synthetic construct
  misc_feature 1..19: note = inhibitory RNA
  source 1..19: mol_type = other RNA
cggaagagaccaagcaaaa

SEQ ID NO: 1216 - RNA, 21 nt, synthetic construct
  misc_feature 1..21: note = inhibitory RNA
  source 1..21: mol_type = other RNA
ttttgcttggtctcttccgtt

SEQ ID NO: 1217 - RNA, 19 nt, synthetic construct
  misc_feature 1..19: note = inhibitory RNA
  source 1..19: mol_type = other RNA
ggaaagaaggatggacaaa

SEQ ID NO: 1218 - RNA, 21 nt, synthetic construct
  misc_feature 1..21: note = inhibitory RNA
  source 1..21: mol_type = other RNA
tttgtccatccttctttcctt

SEQ ID NO: 1219 - RNA, 19 nt, synthetic construct
  misc_feature 1..19: note = inhibitory RNA
  source 1..19: mol_type = other RNA
gcataaaggccttcataga

SEQ ID NO: 1220 - RNA, 21 nt, synthetic construct
  misc_feature 1..21: note = inhibitory RNA
  source 1..21: mol_type = other RNA
tctatgaaggcctttatgctt

SEQ ID NO: 1221 - RNA, 19 nt, synthetic construct
  misc_feature 1..19: note = inhibitory RNA
  source 1..19: mol_type = other RNA
ctacgaaggtcatgaacat

SEQ ID NO: 1222 - RNA, 21 nt, synthetic construct
  misc_feature 1..21: note = inhibitory RNA
  source 1..21: mol_type = other RNA
atgttcatgaccttcgtagtt

SEQ ID NO: 1223 - RNA, 19 nt, synthetic construct
  misc_feature 1..19: note = inhibitory RNA
  source 1..19: mol_type = other RNA
cctctggagtagatagata

SEQ ID NO: 1224 - RNA, 21 nt, synthetic construct
  misc_feature 1..21: note = inhibitory RNA
  source 1..21: mol_type = other RNA
tatctatctactccagaggtt

SEQ ID NO: 1225 - RNA, 19 nt, synthetic construct
  misc_feature 1..19: note = inhibitory RNA
  source 1..19: mol_type = other RNA
gagaaagcagtgatggtaa

SEQ ID NO: 1226 - RNA, 21 nt, synthetic construct
  misc_feature 1..21: note = inhibitory RNA
  source 1..21: mol_type = other RNA
ttaccatcactgctttctctt

SEQ ID NO: 1227 - RNA, 19 nt, synthetic construct
  misc_feature 1..19: note = inhibitory RNA
  source 1..19: mol_type = other RNA
gaggatggtatgcgggata

SEQ ID NO: 1228 - RNA, 21 nt, synthetic construct
  misc_feature 1..21: note = inhibitory RNA
  source 1..21: mol_type = other RNA
tatcccgcataccatcctctt

SEQ ID NO: 1229 - RNA, 19 nt, synthetic construct
  misc_feature 1..19: note = inhibitory RNA
  source 1..19: mol_type = other RNA
ccagaaggaatgagaatca

SEQ ID NO: 1230 - RNA, 21 nt, synthetic construct
  misc_feature 1..21: note = inhibitory RNA
  source 1..21: mol_type = other RNA
tgattctcattccttctggtt

SEQ ID NO: 1231 - RNA, 19 nt, synthetic construct
  misc_feature 1..19: note = inhibitory RNA
  source 1..19: mol_type = other RNA
ccaagtacgagatgaacaa

SEQ ID NO: 1232 - RNA, 21 nt, synthetic construct
  misc_feature 1..21: note = inhibitory RNA
  source 1..21: mol_type = other RNA
ttgttcatctcgtacttggtt

SEQ ID NO: 1233 - RNA, 19 nt, synthetic construct
  misc_feature 1..19: note = inhibitory RNA
  source 1..19: mol_type = other RNA
cagaatctatggtggttta

SEQ ID NO: 1234 - RNA, 20 nt, synthetic construct
  misc_feature 1..20: note = inhibitory RNA
  source 1..20: mol_type = other RNA
aaaccaccatagattctgtt

SEQ ID NO: 1235 - RNA, 21 nt, synthetic construct
  misc_feature 1..21: note = inhibitory RNA
  source 1..21: mol_type = other RNA
gaagacataattccagaagaa

SEQ ID NO: 1236 - RNA, 23 nt, synthetic construct
  misc_feature 1..23: note = inhibitory RNA
  source 1..23: mol_type = other RNA
ttcttctggaattatgtcttctt

SEQ ID NO: 1237 - RNA, 21 nt, synthetic construct
  misc_feature 1..21: note = inhibitory RNA
  source 1..21: mol_type = other RNA
cggggaaacggtggtggagaa

SEQ ID NO: 1238 - RNA, 23 nt, synthetic construct
  misc_feature 1..23: note = inhibitory RNA
  source 1..23: mol_type = other RNA
ttctccaccaccgtttccccgtt

SEQ ID NO: 1239 - RNA, 21 nt, synthetic construct
  misc_feature 1..21: note = inhibitory RNA
  source 1..21: mol_type = other RNA
ctccgcagagtttgaggtgaa

SEQ ID NO: 1240 - RNA, 23 nt, synthetic construct
  misc_feature 1..23: note = inhibitory RNA
  source 1..23: mol_type = other RNA
ttcacctcaaactctgcggagtt

SEQ ID NO: 1241 - RNA, 21 nt, synthetic construct
  misc_feature 1..21: note = inhibitory RNA
  source 1..21: mol_type = other RNA
gagttgaaagaaccagagaaa

SEQ ID NO: 1242 - RNA, 23 nt, synthetic construct
  misc_feature 1..23: note = inhibitory RNA
  source 1..23: mol_type = other RNA
tttctctggttctttcaactctt

SEQ ID NO: 1243 - RNA, 21 nt, synthetic construct
  misc_feature 1..21: note = inhibitory RNA
  source 1..21: mol_type = other RNA
agagtttgaggtgaaggaata

SEQ ID NO: 1244 - RNA, 23 nt, synthetic construct
  misc_feature 1..23: note = inhibitory RNA
  source 1..23: mol_type = other RNA
tattccttcacctcaaactcttt

SEQ ID NO: 1245 - RNA, 21 nt, synthetic construct
  misc_feature 1..21: note = inhibitory RNA
  source 1..21: mol_type = other RNA
gagggtggaactgttgcataa

SEQ ID NO: 1246 - RNA, 23 nt, synthetic construct
  misc_feature 1..23: note = inhibitory RNA
  source 1..23: mol_type = other RNA
ttatgcaacagttccaccctctt

SEQ ID NO: 1247 - RNA, 21 nt, synthetic construct
  misc_feature 1..21: note = inhibitory RNA
  source 1..21: mol_type = other RNA
atggacaaagctggtcagtac

SEQ ID NO: 1248 - RNA, 23 nt, synthetic construct
  misc_feature 1..23: note = inhibitory RNA
  source 1..23: mol_type = other RNA
gtactgaccagctttgtccattt

SEQ ID NO: 1249 - RNA, 21 nt, synthetic construct
  misc_feature 1..21: note = inhibitory RNA
  source 1..21: mol_type = other RNA
caggagtgaattggaggaaga

SEQ ID NO: 1250 - RNA, 23 nt, synthetic construct
  misc_feature 1..23: note = inhibitory RNA
  source 1..23: mol_type = other RNA
tcttcctccaattcactcctgtt

SEQ ID NO: 1251 - RNA, 21 nt, synthetic construct
  misc_feature 1..21: note = inhibitory RNA
  source 1..21: mol_type = other RNA
gaccatagaagagttgaaaga

SEQ ID NO: 1252 - RNA, 23 nt, synthetic construct
  misc_feature 1..23: note = inhibitory RNA
  source 1..23: mol_type = other RNA
tctttcaactcttctatggtctt

SEQ ID NO: 1253 - RNA, 21 nt, synthetic construct
  misc_feature 1..21: note = inhibitory RNA
  source 1..21: mol_type = other RNA
caacatagagctgttggatga

SEQ ID NO: 1254 - RNA, 23 nt, synthetic construct
  misc_feature 1..23: note = inhibitory RNA
  source 1..23: mol_type = other RNA
tcatccaacagctctatgttgtt

SEQ ID NO: 1255 - RNA, 21 nt, synthetic construct
  misc_feature 1..21: note = inhibitory RNA
  source 1..21: mol_type = other RNA
atcggaagagaccaagcaaaa

SEQ ID NO: 1256 - RNA, 23 nt, synthetic construct
  misc_feature 1..23: note = inhibitory RNA
  source 1..23: mol_type = other RNA
ttttgcttggtctcttccgattt

SEQ ID NO: 1257 - RNA, 21 nt, synthetic construct
  misc_feature 1..21: note = inhibitory RNA
  source 1..21: mol_type = other RNA
atggaaagaaggatggacaaa

SEQ ID NO: 1258 - RNA, 23 nt, synthetic construct
  misc_feature 1..23: note = inhibitory RNA
  source 1..23: mol_type = other RNA
tttgtccatccttctttccattt

SEQ ID NO: 1259 - RNA, 21 nt, synthetic construct
  misc_feature 1..21: note = inhibitory RNA
  source 1..21: mol_type = other RNA
ctgcataaaggccttcataga

SEQ ID NO: 1260 - RNA, 23 nt, synthetic construct
  misc_feature 1..23: note = inhibitory RNA
  source 1..23: mol_type = other RNA
tctatgaaggcctttatgcagtt

SEQ ID NO: 1261 - RNA, 21 nt, synthetic construct
  misc_feature 1..21: note = inhibitory RNA
  source 1..21: mol_type = other RNA
ctctacgaaggtcatgaacat

SEQ ID NO: 1262 - RNA, 23 nt, synthetic construct
  misc_feature 1..23: note = inhibitory RNA
  source 1..23: mol_type = other RNA
atgttcatgaccttcgtagagtt

SEQ ID NO: 1263 - RNA, 21 nt, synthetic construct
  misc_feature 1..21: note = inhibitory RNA
  source 1..21: mol_type = other RNA
ggcctctggagtagatagata

SEQ ID NO: 1264 - RNA, 23 nt, synthetic construct
  misc_feature 1..23: note = inhibitory RNA
  source 1..23: mol_type = other RNA
tatctatctactccagaggcctt

SEQ ID NO: 1265 - RNA, 21 nt, synthetic construct
  misc_feature 1..21: note = inhibitory RNA
  source 1..21: mol_type = other RNA
tggagaaagcagtgatggtaa

SEQ ID NO: 1266 - RNA, 23 nt, synthetic construct
  misc_feature 1..23: note = inhibitory RNA
  source 1..23: mol_type = other RNA
ttaccatcactgctttctccatt

SEQ ID NO: 1267 - RNA, 21 nt, synthetic construct
  misc_feature 1..21: note = inhibitory RNA
  source 1..21: mol_type = other RNA
gtgaggatggtatgcgggata

SEQ ID NO: 1268 - RNA, 23 nt, synthetic construct
  misc_feature 1..23: note = inhibitory RNA
  source 1..23: mol_type = other RNA
tatcccgcataccatcctcactt

SEQ ID NO: 1269 - RNA, 21 nt, synthetic construct
  misc_feature 1..21: note = inhibitory RNA
  source 1..21: mol_type = other RNA
tgccagaaggaatgagaatca

SEQ ID NO: 1270 - RNA, 23 nt, synthetic construct
  misc_feature 1..23: note = inhibitory RNA
  source 1..23: mol_type = other RNA
tgattctcattccttctggcatt

SEQ ID NO: 1271 - RNA, 21 nt, synthetic construct
  misc_feature 1..21: note = inhibitory RNA
  source 1..21: mol_type = other RNA
ctccaagtacgagatgaacaa

SEQ ID NO: 1272 - RNA, 23 nt, synthetic construct
  misc_feature 1..23: note = inhibitory RNA
  source 1..23: mol_type = other RNA
ttgttcatctcgtacttggagtt

SEQ ID NO: 1273 - RNA, 21 nt, synthetic construct
  misc_feature 1..21: note = inhibitory RNA
  source 1..21: mol_type = other RNA
cacagaatctatggtggttta

SEQ ID NO: 1274 - RNA, 23 nt, synthetic construct
  misc_feature 1..23: note = inhibitory RNA
  source 1..23: mol_type = other RNA
taaaccaccatagattctgtgtt

(19) United States
(12) Patent Application Publication
(10) Pub. No.: US 2026/0265755 A1
(43) Pub. Date: Sep. 10, 2026

(54) CONJUGATE COMPRISING A DOUBLE STRANDED RNA MOLECULE LINKED TO A SINGLE STRANDED DNA MOLECULE

(71) Applicant: ARGONAUTE RNA LIMITED, Bristol (GB)

(72) Inventors: Michael KHAN, Bristol (GB); Daniel MITCHELL, Bristol (GB); Johnathan MATLOCK, Bristol (GB)

(21) Appl. No.: 19/164,297

(22) PCT Filed: Mar. 13, 2024
(86) PCT No.: PCT/GB2024/050669

§ 371 Date: Sep. 11, 2025

(30) Foreign Application Priority Data
Mar. 14, 2023 (GB) 2303696.5
May 30, 2023 (GB) 2308055.9

(51) Int. Cl. C12N 15/113; A61K 31/7125; A61K 31/713

(52) U.S. Cl. CPC C12N 15/1137; A61K 31/7125; A61K 31/713; C12N 2310/11; C12N 2310/14; C12N 2310/312; C12N 2310/315; C12N 2310/351; C12Y 304/21104

(57) ABSTRACT

This disclosure relates to a nucleic acid comprising a double stranded RNA molecule comprising sense and antisense strands and further comprising a single stranded DNA molecule covalently linked to at least the 5′ end of either the sense or antisense RNA part of the molecule.

### FIELD OF THE DISCLOSURE

This disclosure relates to an isolated nucleic acid molecule comprising a double stranded RNA molecule comprising sense and antisense strands and further comprising a single stranded DNA molecule covalently linked to at least the 5′ end of either the sense or antisense RNA part of the molecule and wherein said nucleic acid molecule is optionally linked, directly or indirectly, to N-acetylgalactosamine (also referred to as “GalNAc”).

### BACKGROUND TO THE DISCLOSURE

A technique to specifically ablate gene function is through the introduction of double stranded inhibitory RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically, but not exclusively, derived from exons of the gene which is to be ablated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in ablation of the mRNA.

RNAi technology is a promising therapeutic tool. However, siRNA suffers from a lack of stability and cell/tissue targeting. Methods to increase the stability are desirable. US2019085328 discloses siRNA molecules having internal modifications that enhance the stability of siRNA such as sugar modification, base modification and/or backbone modifications including cross linkers, dendrimers, nanoparticles, peptides, organic compounds (e.g., fluorescent dyes), and/or photocleavable compounds. US2009197332 discloses siRNA molecules comprising chemically modified nucleotides that protect the siRNA against degradation. US2016193354 discloses siRNA-conjugate molecules wherein the conjugate comprises a modified and/or natural oligonucleotide, a linker group, sulphur and either hydrogen or a thiol protecting group. However, although the stability of modified siRNA molecules is increased, the synthesis is often difficult and expensive, and moreover the modifications can lead to increased toxicity and adverse side effects which must be controlled when used in the clinic.

The disclosure relates to siRNA silencing of genes associated with the complement system which is part of the innate immunity of an animal. Complement proteins are part of the innate immune response. They perform a range of biological functions such as opsonization (coating foreign pathogens), initiating the membrane attack complex and enhancing inflammation, by activating different pathways: classical, lectin, and alternate. Complement pathways converge to a common pathway that causes splitting or activation of C3 to make C3a or C3b, resulting in the formation of various bioactive molecules such as C5a and C5b. The over activation of the complement system can have serious clinical outcomes such as in sepsis in response to a microbial pathogen such as a virus or bacterial pathogen.

### STATEMENTS OF THE INVENTION

The present disclosure relates to a nucleic acid molecule comprising a double stranded inhibitory RNA that is modified by the inclusion of a short DNA part linked to at least the 5′ end of either the sense or antisense inhibitory RNA and which forms a hairpin structure (a “crook”) and further optionally comprises N-acetylgalactosamine. The position of N-acetylgalactosamine can be varied in the nucleic acid molecule. N-acetylgalactosamine can be linked, directly or indirectly, to the DNA part or the RNA part.

The nucleic acid molecules according to the invention have improved stability without the need for modified bases and/or sugars comprising the inhibitory RNA and uses predominantly natural bases/sugars. N-acetylgalactosamine allows specific targeting of siRNA to the liver, providing highly efficacious gene silencing.

According to an aspect of the invention there is provided a nucleic acid molecule comprising
a first part that comprises a double stranded inhibitory ribonucleic acid (RNA) molecule comprising a sense strand and an antisense strand designed with reference to a nucleotide sequence comprising a gene to be silenced; and
a second part that comprises a single stranded deoxyribonucleic acid (DNA) molecule, wherein the 3′ end of said single stranded DNA molecule is covalently linked to at least the 5′ end of the sense strand of the double stranded inhibitory RNA molecule or wherein the 3′ end of the single stranded DNA molecule is covalently linked to at least the 5′ of the antisense strand of the double stranded inhibitory RNA molecule; and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

According to an aspect of the invention there is provided a nucleic acid molecule comprising:
a first part that comprises a double stranded inhibitory ribonucleic acid (RNA) molecule comprising a sense strand and an antisense strand; and
a second part that comprises a single stranded deoxyribonucleic acid (DNA) molecule, wherein the 3′ end of said single stranded DNA molecule is covalently linked to the 5′ end of the sense strand of the double stranded inhibitory RNA molecule or wherein the 3′ end of the single stranded DNA molecule is covalently linked to the 5′ of the antisense strand of the double stranded inhibitory RNA molecule; characterized in that the double stranded inhibitory RNA comprises a sense nucleotide sequence that encodes a part of a human complement component gene and wherein said single stranded DNA molecule comprises a nucleotide sequence that is adapted over at least part of its length to anneal by complementary base pairing to a part of said single stranded DNA to form a double stranded DNA structure comprising a stem and a loop domain and said nucleic acid molecule is linked to N-acetylgalactosamine and said double stranded inhibitory RNA consists of natural nucleotides.

According to an aspect of the invention there is provided a nucleic acid molecule comprising:
a first part that comprises a double stranded inhibitory ribonucleic acid (RNA) molecule comprising a sense strand and an antisense strand; and
a second part that comprises a single stranded deoxyribonucleic acid (DNA) molecule, wherein the 3′ end of said single stranded DNA molecule is covalently linked to the 5′ end of the sense strand of the double stranded inhibitory RNA molecule or wherein the 3′ end of the single stranded DNA molecule is covalently linked to the 5′ of the antisense strand of the double stranded inhibitory RNA molecule, characterized in that the double stranded inhibitory RNA comprises a sense nucleotide sequence that encodes a part of the human complement component 5 (C5), or polymorphic sequence variant thereof, and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

According to an aspect of the invention there is provided a nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 3′ end of said single stranded DNA molecule is covalently linked to the 5′ end of the sense strand of the double stranded inhibitory RNA molecule or wherein the 3′ end of the single stranded DNA molecule is covalently linked to the 5′ of the antisense strand of the double stranded inhibitory RNA molecule, characterized in that the double stranded inhibitory RNA comprises a sense nucleotide sequence that encodes a part of the human complement component 3 (C3), or polymorphic sequence variant thereof, and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

According to an aspect of the invention there is provided a nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 3′ end of said single stranded DNA molecule is covalently linked to the 5′ end of the sense strand of the double stranded inhibitory RNA molecule or wherein the 3′ end of the single stranded DNA molecule is covalently linked to the 5′ of the antisense strand of the double stranded inhibitory RNA molecule, characterized in that the double stranded inhibitory RNA comprises a sense nucleotide sequence that encodes a part of the human MASP-2 gene, or polymorphic sequence variant thereof, and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

A polymorphic sequence variant varies from a reference sequence by 1, 2 or 3 or more nucleotides.

In a preferred embodiment of the invention the 3′ end of said single stranded DNA molecule is covalently linked to the 5′ end of the sense strand of the double stranded inhibitory RNA molecule.

In an alternative embodiment of the invention the 3′ end of said single stranded DNA molecule is covalently linked to the 5′ end of the antisense strand of the double stranded inhibitory RNA molecule.

In a preferred embodiment of the invention said loop portion comprises a region comprising the nucleotide sequence GNA or GNNA, wherein each N independently represents guanine (G), thymidine (T), adenine (A), or cytosine (C).

In a preferred embodiment of the invention said loop domain comprises G and C nucleotide bases.

In an alternative embodiment of the invention said loop domain comprises the nucleotide sequence GCGAAGC.

In a further preferred embodiment of the invention said single stranded DNA molecule comprises the nucleotide sequence 5′ TCACCTCATCCCGCGAAGC 3′ (SEQ ID NO 1)

In an alternative preferred embodiment of the invention said single stranded DNA molecule comprises the nucleotide sequence 5′ CGAAGCGCCCTACTCCACT 3′ (SEQ ID NO 150).

In an alternative preferred embodiment of the invention said single stranded DNA molecule comprises the nucleotide sequence 5′ GCGAAGCCCCTACTCCACT 3′ (SEQ ID 1155).

In a preferred embodiment of the invention said stem domain comprises 2, 3, 4, 5, 6, 7, 8, 9, 10, 11 or at least 12 nucleotides in length.

The inhibitory RNA molecules comprise or consist of natural nucleotide bases that do not require chemical modification. Moreover, in some embodiments of the invention, wherein the crook DNA molecule is linked to the 5′ end of the sense strand of said double stranded inhibitory RNA, the antisense strand is optionally provided with at least a two-nucleotide base overhang sequence. The two-nucleotide overhang sequence can correspond to nucleotides encoded by the target or are non-encoding. The two-nucleotide overhang can be two nucleotides of any sequence and in any order, for example UU, AA, UA, AU, GG, CC, GC, CG, UG, GU, UC, CU and TT.

In a preferred embodiment of the invention said inhibitory RNA molecule comprises a two-nucleotide overhang comprising or consisting of deoxythymidine dinucleotide (dTdT).

In a preferred embodiment of the invention said dTdT overhang is positioned at the 5′ end of said antisense strand.

In an alternative preferred embodiment of the invention said dTdT overhang is positioned at the 3′ end of said antisense strand.

In a preferred embodiment of the invention said dTdT overhang is positioned at the 5′ end of said sense strand.

In an alternative preferred embodiment of the invention said dTdT overhang is positioned at the 3′ end of said sense strand.

In a preferred embodiment of the invention said sense and/or said antisense strands comprises internucleotide phosphorothioate linkages.

In a preferred embodiment of the invention said sense strand comprises internucleotide phosphorothioate linkages.

Preferably, the 5′ and/or 3′ terminal two nucleotides of said sense strand comprises two internucleotide phosphorothioate linkage.

In a preferred embodiment of the invention said antisense strand comprises internucleotide phosphorothioate linkages.

Preferably, the 5′ and/or 3′ terminal two nucleotides of said antisense strand comprises two internucleotide phosphorothioate linkages.

In an alternative preferred embodiment of the invention said single stranded DNA molecule comprises one or more internucleotide phosphorothioate linkages.

In a preferred embodiment of the invention said nucleic acid molecule comprises a vinylphosphonate modification.

In a preferred embodiment of the invention said vinylphosphonate modification is to the 5′ terminal phosphate of said sense RNA strand.

In a preferred embodiment of the invention said vinylphosphonate modification is to the 5′ terminal phosphate of said antisense RNA strand.

In a preferred embodiment of the invention said double stranded inhibitory RNA molecule comprises 15 to 40 contiguous nucleotides in length.

In a preferred embodiment of the invention said double stranded inhibitory RNA molecule comprises at least 19 contiguous nucleotides in length.

In a preferred embodiment of the invention said double stranded inhibitory RNA molecule comprises at least 21 contiguous nucleotides in length.

In a preferred embodiment of the invention said double stranded inhibitory RNA molecule comprises 21 to 23 contiguous nucleotides.

In a preferred embodiment of the invention N-acetylgalactosamine is monovalent, divalent, trivalent or tetravalent.

In a further embodiment of the invention N-acetylgalactosamine is linked to either the antisense part of said inhibitory RNA or the sense part of said inhibitory RNA.

Preferably, N-acetylgalactosamine is linked to the 5′ terminus is of said sense RNA.

In an alternative embodiment of the invention N-acetylgalactosamine is linked to the 3′ terminus of said sense RNA.

In an alternative preferred embodiment of the invention said N-acetylgalactosamine is linked to the 3′ terminus of said antisense RNA.

In a preferred embodiment of the invention said nucleic acid molecule is covalently linked to a molecule comprising the structure:

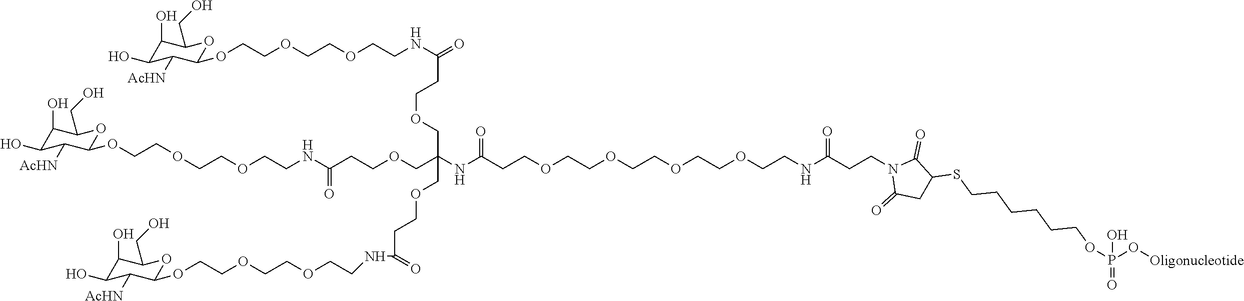

In an alternative embodiment of the invention said nucleic acid molecule is covalently linked to a molecule comprising the structure:

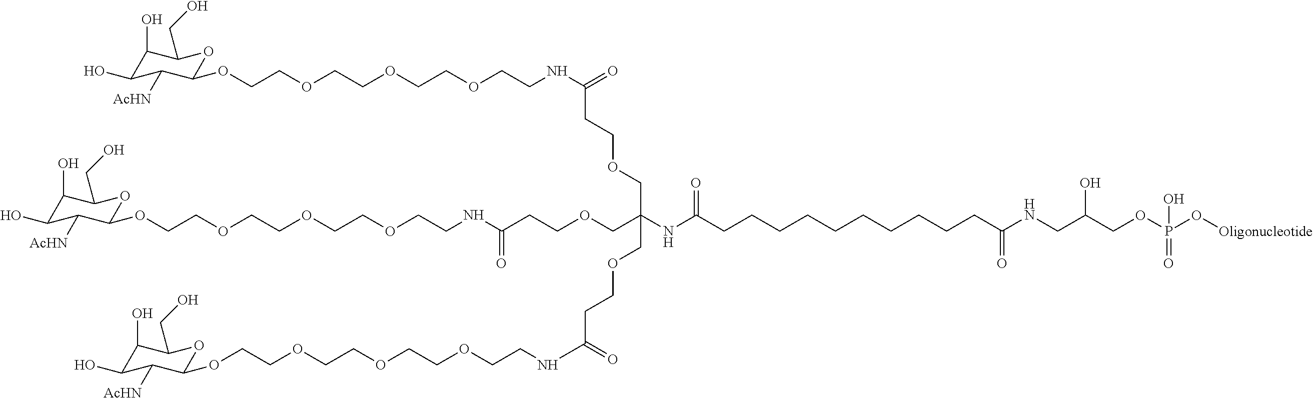

In an alternative embodiment of the invention said nucleic acid molecule is covalently linked to a molecule comprising the structure:

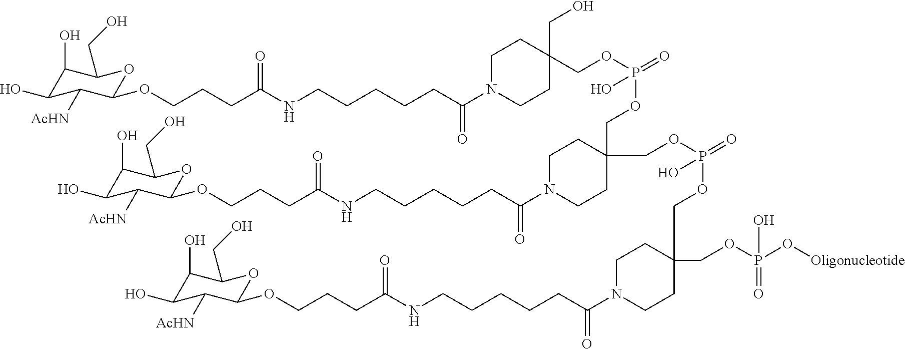

In an alternative embodiment of the invention said nucleic acid molecule is covalently linked to a molecule comprising the structure:

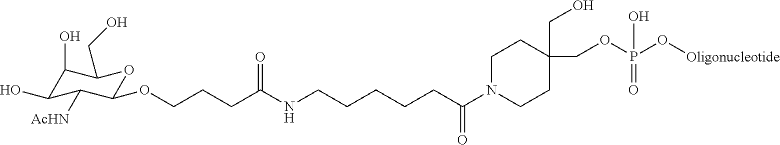

Inhibitory nucleic acid molecules comprising RNA sequences directed to silencing complement associated genes are known.

In a preferred embodiment of the invention said C5 double stranded inhibitory RNA molecule comprises or consists of between 19 and 23 contiguous nucleotides of the sense nucleotide sequence set forth in SEQ ID NO: 67.

In an embodiment of the invention said C5 double stranded inhibitory RNA molecule comprises a nucleotide sequence selected from the group consisting of: SEQ ID NO: 119, 120, 121, 122, 123, 124, 125, 126, 127, 128, 129, 130, 131, 132, 133, 134, 135, 136, 137 and 138.

In an embodiment of the invention said C5 double stranded inhibitory RNA molecule comprises a nucleotide sequence selected from the group consisting of: SEQ ID NO: 109, 110, 111, 112, 113, 114, 115, 116, 117 and 118.

In an embodiment of the invention said C5 double stranded inhibitory RNA molecule comprises a nucleotide sequence selected from the group consisting of: SEQ ID NO: 139, 140, 141, 142, 143, 144, 145, 146, 147 and 148.

In an embodiment of the invention said C5 double stranded inhibitory RNA molecule comprises an antisense nucleotide sequence selected from the group consisting of: SEQ ID NO: 31, 32, 33, 34, 35, 36, 37, 38, 39 and 40.

In an alternative embodiment of the invention said C5 double stranded inhibitory RNA molecule comprises a sense nucleotide sequence selected from the group consisting of: SEQ ID NO: 21, 22, 23, 24, 25, 26, 27, 28, 29 and 30.

In a preferred embodiment of the invention said C3 double stranded inhibitory RNA molecule comprises or consists of between 19 and 23 contiguous nucleotides of the sense nucleotide sequence set forth in SEQ ID NO: 66.

In an embodiment of the invention said C3 double stranded inhibitory RNA molecule comprises a nucleotide sequence selected from the group consisting of: SEQ ID NO: 79, 80, 81, 82, 83, 84, 85, 86, 87, 88, 89, 90, 91, 92, 93, 94, 95, 96, 97, 98 and 99.

In an embodiment of the invention said C3 double stranded inhibitory RNA molecule comprises a nucleotide sequence selected from the group consisting of: SEQ ID NO: 69, 70, 71, 72, 73, 74, 75, 76, 77 and 78.

In an embodiment of the invention said C3 double stranded inhibitory RNA molecule comprises a nucleotide sequence selected from the group consisting of: SEQ ID NO: 100, 101, 102, 103, 104, 105, 106, 107 and 108.

In an embodiment of the invention said C3 double stranded inhibitory RNA molecule comprises an antisense nucleotide sequence selected from the group consisting of: SEQ ID NO: 11, 12, 13, 14, 15, 16, 17, 18, 19 and 20.

In an alternative embodiment of the invention said C3 double stranded inhibitory RNA molecule comprises a sense nucleotide sequence selected from the group consisting of: 61, 2, 3, 4, 5, 6, 7, 8, 9 and 10.

In a preferred embodiment of the invention said C3 double stranded inhibitory RNA molecule comprises sense and antisense pairs as disclosed in Table 1.

In an preferred embodiment of the invention said C3 double stranded inhibitory RNA molecule comprises a sense nucleotide sequence selected from the group consisting of: 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271 and 1273.

In an alternative embodiment of the invention said C3 double stranded inhibitory RNA molecule comprises an antisense nucleotide sequence selected from the group consisting of: 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272 and 1274.

In a preferred embodiment of the invention said double stranded inhibitory RNA molecule comprises or consists of between 19 and 23 contiguous nucleotides of the sense nucleotide sequence set forth in SEQ ID NO: 68 (MASP2).

In an embodiment of the invention said MASP2 double stranded inhibitory RNA molecule comprises an antisense nucleotide sequence selected from the group consisting of: 51, 52, 53, 54, 55, 56, 57, 58, 59 or 60.

In an alternative embodiment of the invention said MASP2 double stranded inhibitory RNA molecule comprises a sense nucleotide sequence selected from the group consisting of: SEQ ID NO: 41, 42, 43, 44, 45, 46, 47, 48, 49 and 50.

WO2020/104669, the content of which is incorporated by reference in its entirety, discloses silencing of complement gene C3 and their use in the treatment of complement associated diseases such as C3 glomerulopathy, lupus nephritis and myasthenia gravis. Examples include SEQ ID NO 1156.

WO2021/037941 the content of which is incorporated by reference in its entirety, discloses further siRNA sequences that silence expression of C3. Examples include SEQ ID NO 1157, 1158, 1159, 1160, 1161, 1162, 1163 and 1164.

WO2021/081026, the content of which is incorporated by reference in its entirety, is a further disclosure directed to C3 siRNAs such as SEQ ID NO 1165 and 1166 which have use in the treatment of C3 associated diseases and pathologies.

WO2023/152286, the content of which is incorporated by reference in its entirety, discloses an alternative complement target MASP2.

WO2023/245126, the content of which is incorporated by reference in its entirety, is a further alternative approach to modulate complement expression and targets C5 and discloses siRNA sequences such as SEQ ID NO 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190 and 1191.

In a preferred embodiment of the invention said MASP-2 double stranded inhibitory RNA molecule comprises sense and antisense pairs as disclosed in Table 4.

According to an aspect of the invention there is provided a nucleic acid molecule comprising:
a first part that comprises a double stranded inhibitory ribonucleic acid (RNA) molecule comprising a sense strand and an antisense strand comprising a nucleotide sequence selected from the group SEQ ID NO: 62, 63, 64, 65, 149 and 151 to 1154; and
a second part that comprises a single stranded deoxyribonucleic acid (DNA) molecule, wherein the 3′ end of said single stranded DNA molecule is covalently linked to at least the 5′ end of the sense strand of the double stranded inhibitory RNA molecule or wherein the 3′ end of the single stranded DNA molecule is covalently linked to at least the 5′ of the antisense strand of the double stranded inhibitory RNA molecule; characterized in that the double stranded inhibitory RNA comprises a sense nucleotide sequence that encodes a part of the human complement component gene and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

According to a further aspect of the invention there is provided a pharmaceutical composition comprising at least one nucleic acid molecule according to the invention.

In a preferred embodiment of the invention said composition further includes a pharmaceutical carrier and/or excipient.

When administered the compositions of the present invention are administered in pharmaceutically acceptable preparations. Such preparations may routinely contain pharmaceutically acceptable concentrations of salt, buffering agents, preservatives, compatible carriers and optionally other therapeutic agents, such as cholesterol lowering agents, which can be administered separately from the nucleic acid molecule according to the invention or in a combined preparation if a combination is compatible.

The combination of a nucleic acid according to the invention and the other, different therapeutic agent is administered as simultaneous, sequential, or temporally separate dosages.

The therapeutics of the invention can be administered by any conventional route, including injection or by gradual infusion over time. The administration may, for example, be oral, intravenous, intraperitoneal, intramuscular, intracavity, subcutaneous, transdermal or transepithelial.

The compositions of the invention are administered in effective amounts. An “effective amount” is that amount of a composition that alone, or together with further doses, produces the desired response. In the case of treating a disease, such as cardiovascular disease or infection, the desired response is inhibiting or reversing the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of a nucleic acid molecule according to the invention for producing the desired response in a unit of weight or volume suitable for administration to a patient. The response can, for example, be measured by determining regression of cardiovascular disease and decrease of disease symptoms etc.

The doses of the nucleic acid molecule according to the invention administered to a subject can be chosen in accordance with different parameters, in particular in accordance with the mode of administration used and the state of the subject. Other factors include the desired period of treatment. If a response in a subject is insufficient at the initial doses applied, higher doses (or effectively higher doses by a different, more localized delivery route) may be employed to the extent that patient tolerance permits. It will be apparent that the method of detection of the nucleic acid according to the invention facilitates the determination of an appropriate dosage for a subject in need of treatment.

In general, doses of the nucleic acid molecules herein disclosed of between 0.1 mg/kg to 25 mg/kg generally will be formulated and administered according to standard procedures. Preferably doses can range from 0.1 mg/kg to 5 mg/kg or 0.5 mg/kg to 5 mg/kg. Other protocols for the administration of compositions will be known to one of ordinary skill in the art, in which the dose amount, schedule of injections, sites of injections, mode of administration and the like vary from the foregoing. The administration of compositions to mammals other than humans, (e.g., for testing purposes or veterinary therapeutic purposes), is carried out under substantially the same conditions as described above. A subject, as used herein, is a mammal, preferably a human, and including a nonhuman primate, cow, horse, pig, sheep, goat, dog, cat or rodent.

When administered, the pharmaceutical preparations of the invention are applied in pharmaceutically acceptable amounts and in pharmaceutically acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents e.g. statins. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

Compositions may be combined, if desired, with a pharmaceutically acceptable carrier. The term “pharmaceutically acceptable carrier” as used herein means one or more compatible solid or liquid fillers, diluents or encapsulating substances which are suitable for administration into a human. The term “pharmaceutically acceptable carrier” in this context denotes an organic or inorganic ingredient, natural or synthetic, with which the active ingredient is combined to facilitate, for example, solubility and/or stability. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including acetic acid in a salt; citric acid in a salt; boric acid in a salt; and phosphoric acid in a salt. The pharmaceutical compositions also may contain, optionally, suitable preservatives.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product. Compositions suitable for oral administration may be presented as discrete units, such as capsules, tablets, lozenges, each containing a predetermined amount of the active compound.

Compositions suitable for parenteral administration conveniently comprise a sterile aqueous or non-aqueous preparation of nucleic acid, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, PA.

According to an aspect of the invention there is provided a method for inhibiting the expression of a gene in a liver cell comprising administering a nucleic acid molecule or composition according to the invention to a subject.

According to further aspect of the invention there is provided a method for delivery a nucleic acid molecule or composition according to the invention to a liver cell comprising administering an effective amount of the nucleic acid or a composition comprising a nucleic acid to a subject.

In a preferred method of the invention said subject is a human subject.

In a preferred method of the invention said cell is a hepatocyte.

In a preferred method of the invention said cell is a liver cancer cell. Preferably, said liver cancer cell is a primary liver cancer cell. Alternatively, said liver cancer cell is a secondary liver cancer cell.

As used herein, the term “liver cancer” refers to cells having the capacity for autonomous growth, i.e., an abnormal state or condition characterized by rapidly proliferating cell growth. The term is meant to include all types of liver cancerous growths or oncogenic processes, including metastatic liver cancer.

In an alternative method of the invention said cell is a virally infected liver cell.

In a preferred method of the invention said virally infected liver cell is a hepatitis A, hepatitis B hepatitis C, hepatitis D or hepatitis E infected liver cell.

According to a further aspect of the invention there is provided an effective amount of a nucleic acid molecule or pharmaceutical composition according to the invention for use in the treatment of a disease or condition that would benefit from inhibition of complement activation.

According to a further aspect of the invention there is provided an effective amount of a nucleic acid molecule or pharmaceutical composition according to the invention for use in the treatment of a disease or condition that would benefit from inhibition of complement component 5.

According to a further aspect of the invention there is provided an effective amount of a nucleic acid molecule or pharmaceutical composition according to the invention for use in the treatment of a disease or condition that would benefit from inhibition of complement component 3.

According to a further aspect of the invention there is provided an effective amount of a nucleic acid molecule or pharmaceutical composition according to the invention for use in the treatment of a disease or condition that would benefit from inhibition of MASP2.

In a preferred embodiment of the invention said condition is a microbial infection.

In a preferred embodiment of the invention said microbial infection is the result of a viral infection.

In an alternative embodiment of the invention said microbial infection is the result of a bacterial infection.

In a preferred embodiment of the invention said disease or condition is an inflammatory disease or condition.

In a preferred embodiment of the invention said inflammatory disease or condition is selected from the group consisting of: arthritis, nephritis and vasculitis.

In a preferred embodiment of the invention said disease or condition is an autoimmune disease or condition.

In a preferred embodiment of the invention of the invention said disease or condition results in sepsis.

In a preferred embodiment of the invention said disease or condition is acute lung injury.

In a preferred embodiment of the invention said disease or condition is Acute Respiratory Distress Syndrome.

Throughout the description and claims of this specification, the words “comprise” and “contain” and variations of the words, for example “comprising” and “comprises”, means “including but not limited to” and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with an aspect, embodiment or example of the invention are to be understood to be applicable to any other aspect, embodiment or example described herein unless incompatible therewith.

An embodiment of the invention will now be described by example only and with reference to the following figures:

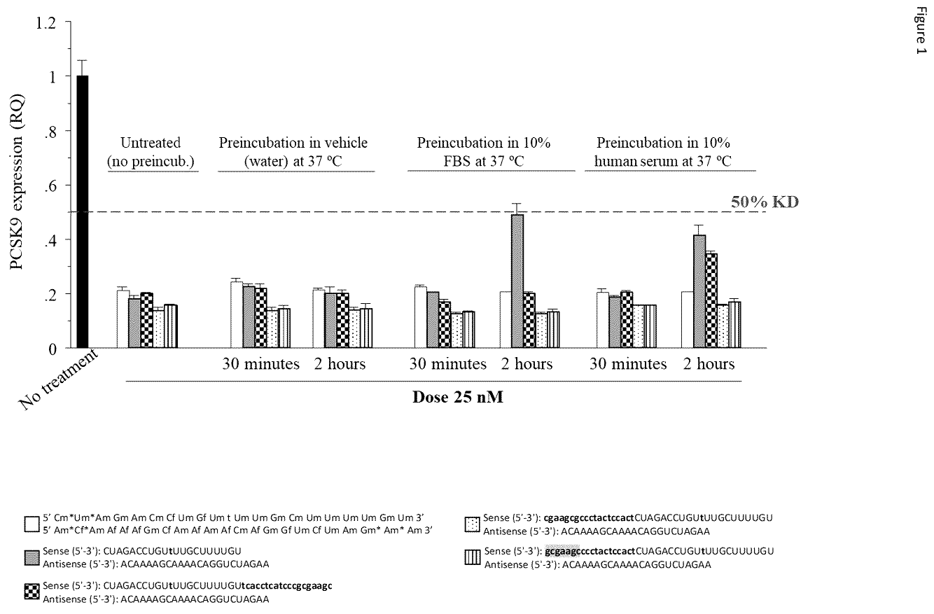

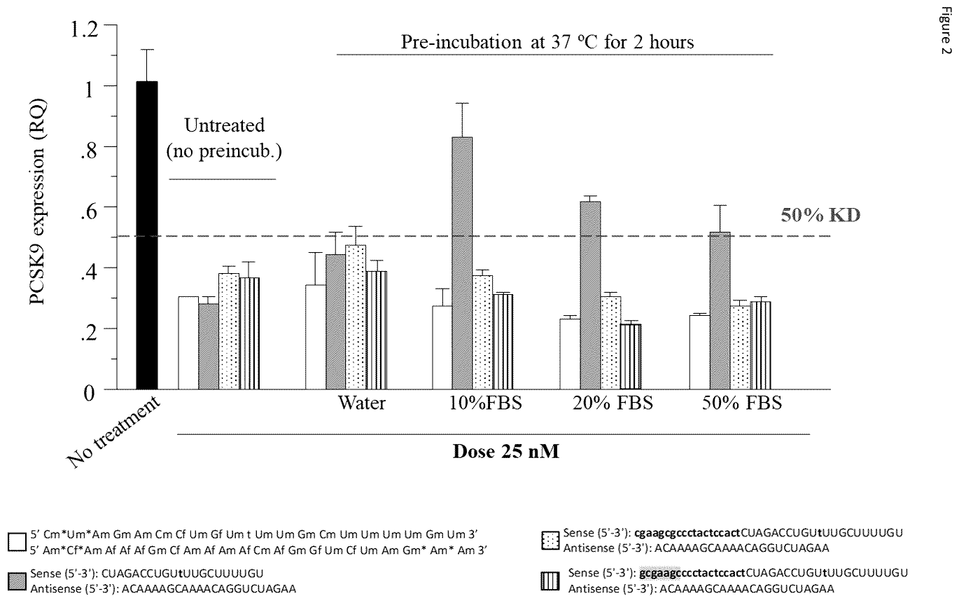

FIG. 1 Serum stability assays showing target PCSK9 mRNA levels in HepG2 cells following transfection of siRNA compounds. HepG2 cells were transfected with the following siRNAs after 30 mins or 2 hr incubation at 37 C in water, 10% FBS or 10% human serum: modified Inclisiran [white bar], unmodified ‘Inclisiran’ with no Crook [grey bar], unmodified Inclisiran with 3′SS Crook [hatched bar], unmodified Inclisiran with 5′SS ‘reversed hairpin’ Crook [spotted bar], or unmodified Inclisiran with 5′SS Crook [hatched bar]. PCSK9 mRNA levels were quantified by RT-qPCR analysis. Controls include ‘no siRNA’ treatment [black bar];

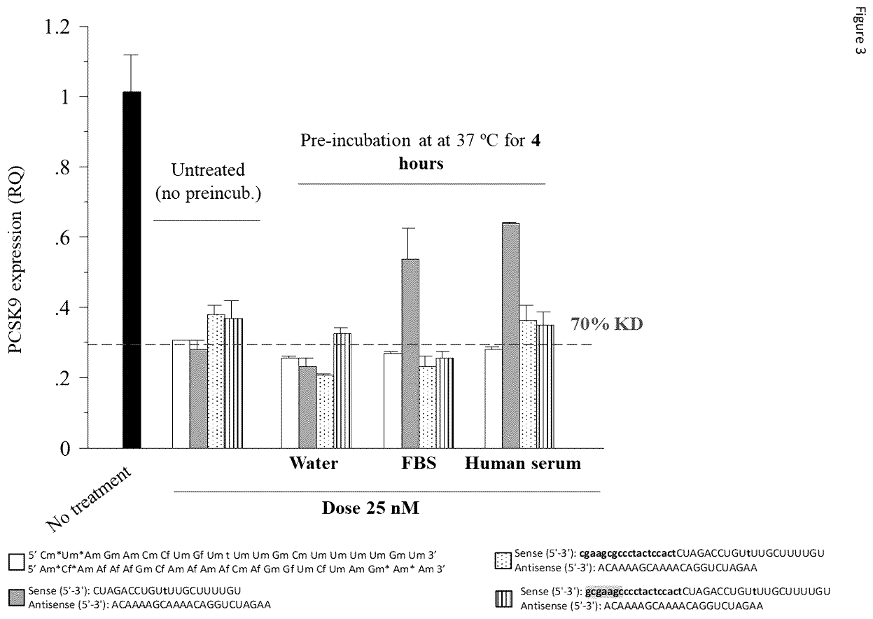

FIG. 2 Serum stability assays showing target PCSK9 mRNA levels in HepG2 cells following transfection of siRNA compounds. HepG2 cells were transfected with the following siRNAs after a 2 hr incubation at 37 C in water, 10%, 20% or 50% FBS: modified Inclisiran [white bar], unmodified ‘Inclisiran’ with no Crook [grey bar], unmodified Inclisiran with 5′SS ‘reversed hairpin’ Crook [spotted bar], or unmodified Inclisiran with 5′SS Crook [striped bar]. PCSK9 mRNA levels were quantified by RT-qPCR analysis. Controls include ‘no siRNA’ pre-treatment [black bar];

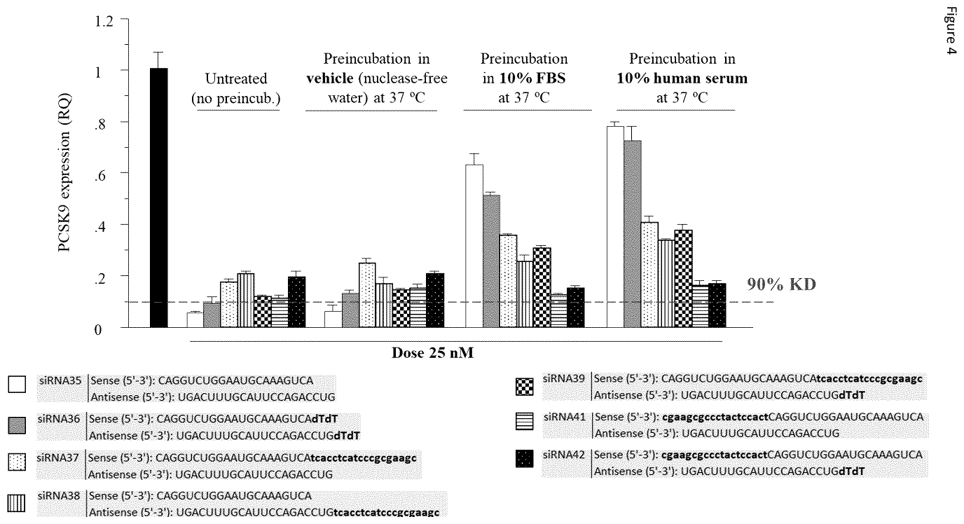

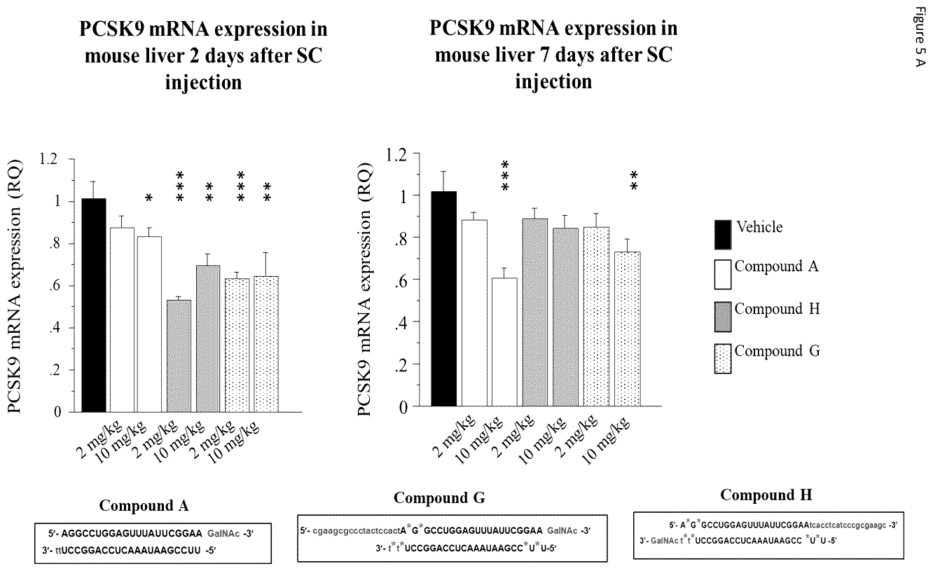

FIG. 3 Serum stability assays showing target PCSK9 mRNA levels in HepG2 cells following transfection of siRNA compounds. HepG2 cells were transfected with the following siRNAs after a 4-hr incubation at 37 C in water, 10% FBS or 10% human serum: modified Inclisiran [white bar], unmodified ‘Inclisiran’ with no Crook [grey bar], unmodified Inclisiran with 5′SS ‘reversed hairpin’ Crook [spotted bar], or unmodified Inclisiran with 5′SS Crook [striped bar]. PCSK9 mRNA levels were quantified by RT-qPCR analysis. Controls include ‘no siRNA’ pre-treatment [black bar];

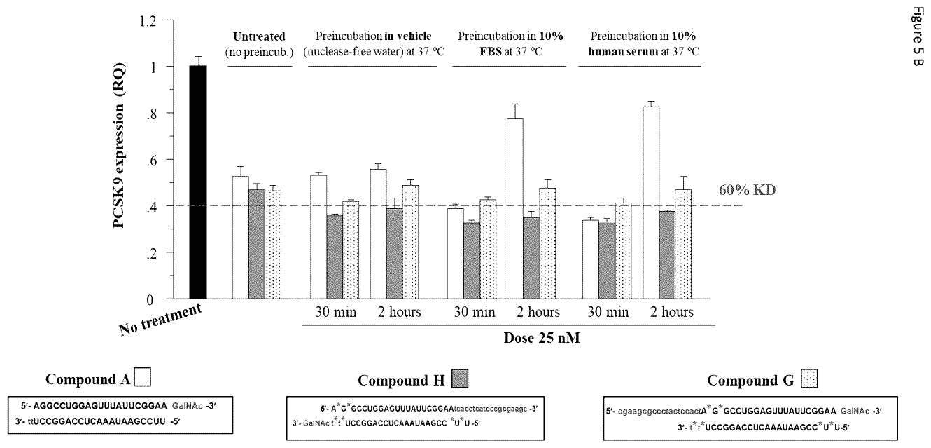

FIG. 4 Serum stability assays showing target PCSK9 mRNA levels in HepG2 cells following transfection of siRNA (termed PC8-PC18) compounds. HepG2 cells were transfected with the following unmodified PC8-18 siRNAs after a 2-hr incubation at 37 C in water, 10% FBS or 10% human serum: siRNA35 with no Crook [white bar], siRNA36 with no Crook but including dTdT overhangs on 3′ SS & 3′ AS [grey bar], siRNA37 with Crook on 3′ SS [spotted bar], siRNA38 with Crook on 3′ AS [vertical striped bar], siRNA39 with Crook on 3′ SS and dTdT overhang on 3′ AS [hatched bar], siRNA41 with 5′SS ‘reversed hairpin’ Crook [horizontal stripe bar], or siRNA42 with Crook on 5′ SS and dTdT overhang on 3′ AS [spots of black background bar]. PCSK9 mRNA levels were quantified by RT-qPCR analysis. Controls include ‘no siRNA’ pre-treatment [black bar];

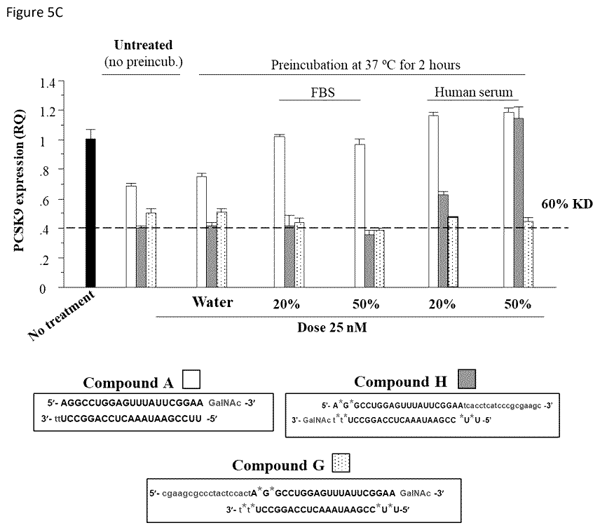

FIG. 5A In vivo silencing of liver PCSK9 mRNA following administration of unmodified siRNA compounds (termed PC2-PC12) Groups of 5 mice for each treatment group were injected subcutaneously (SC) with either vehicle [black bar], compound A (no Crook; white bar), compound G (Crook on 5′ end of sense strand (SS); spotted bar), or compound H (Crook on 3′ end of SS; grey bar). Each compound was given at either 2 mg/kg or 10 mg/kg, and following sacrifice, levels of liver PCSK9 mRNA by RT-qPCR were measured at two time points (day 2 and day 7)

FIG. 5B Serum stability assays showing target PCSK9 mRNA levels in HepG2 cells following transfection of siRNA compounds A, G and H, used in mouse in vivo study (FIG. 5A). HepG2 cells were transfected with siRNA compounds A, G, or H after 30 min or 2 hr incubation at 37 C in water, 10% FBS or 10% human serum: compound A (no Crook; white bar), compound G (Crook on 5′ end of sense strand (SS); spotted bar), or compound H (Crook on 3′ end of SS; grey bar). PCSK9 mRNA levels were quantified by RT-qPCR analysis. Controls include ‘no siRNA’ [black bar], and ‘no serum’ pre-treatment; and

FIG. 5C Serum stability assays showing target PCSK9 mRNA levels in HepG2 cells following transfection of siRNA compounds A, G and H, used in mouse in vivo study (FIG. 5A). HepG2 cells were transfected with siRNA compounds A, G, or H after a 2 hr incubation at 37 C in water, 20% or 50% human serum: compound A (no Crook; white bar), compound G (Crook on 5′ end of sense strand (SS); spotted bar), or compound H (Crook on 3′ end of SS; grey bar). PCSK9 mRNA levels were quantified by RT-qPCR analysis. Controls include ‘no siRNA’ [black bar], and ‘no serum’ pre-treatment.

### MATERIALS AND METHODS

### HepG2 Reverse Transfection

Duplex siRNAs synthesized by Bio-Synthesis (Lewisville, TX) (Table 1), were resuspended in Nuclease-free water (Invitrogen™ AM9932) to generate a stock solution of 10 μM. For serum stability assay, stock siRNAs were incubated at 37° C. in vehicle (nuclease-free water), 10% fetal bovine serum (FBS) or 10% human serum for 30 minutes, 2, 4 or 8 hours or at 20% and 50% FBS or human serum for 2 hours only. After pre-incubation in serum or vehicle, siRNAs were transfected into HepG2 cells in a 384-well plate (Thermo Scientific™ 164688) at a concentration of 25 nM using 0.15 μL of Lipofectamine RNAiMAX (Invitrogen™13778075) per well. Triplicate technical replicates were seeded per assay condition. Transfected cells were incubated for 72 or 48 hours at 37° C., 5% CO₂ concentration and 95% relative humidity. Cells receiving no siRNA treatment were used as control.

### Duplex RT-qPCR

### PCSK9

Cells were processed for RT-qPCR read-out using the Cells-to-CT 1-step TaqMan Kit (Invitrogen™ A25603). Briefly, cells were washed with 50 μl ice-cold PBS and then lysed in 20 μl Lysis solution containing DNase I. After 5 min, lysis was stopped by addition of 2 μl 10 STOP Solution for 2 min. For the RT-qPCR analysis, 1 μl of lysate was dispensed per well into 96-well PCR plate as template in an 20 μl RT-qPCR reaction volume. RT-qPCR was performed using the TaqMan® 1-Step qRT-PCR Mix which came with the Cells-to-CT 1-step TaqMan Kit, with TaqMan probes for GAPDH (VIC_PL, Assay Id Hs00266705_g1) and PCSK9 (FAM, Assay Id Hs00545399-m1). RT-qPCR was performed using a QuantStudio 5 thermocycling instrument (Applied BioSystems). Relative quantification was determined using the AACT method, where GAPDH was used as internal control and expression changes normalized to the reference sample (no treated cells).

### Complement 3 (C3)

Cells were processed for RT-qPCR read-out using the Cells-to-CT 1-step TaqMan Kit (Invitrogen™ A25603). Briefly, cells were washed with 50 μL ice-cold PBS and lysed in 20 μl Lysis solution containing DNase I. Lysis was stopped after 5 minutes by addition of 2 μl STOP Solution for 2 min. For the RT-qPCR analysis, 1 μL of lysate was dispensed per well into a 96 -well PCR plate in a 10 μL RT-qPCR reaction volume. RT-qPCR was performed using the 25 TaqMan® 1-Step qRT-PCR Mix from the Cells-to-CT 1-step TaqMan Kit, with TaqMan probes for GAPDH (VIC_PL, Assay Id Mm99999915_g1) or Complement C3 (FAM, Assay Id Mm01232779_m1). RT-qPCR was performed using a QuantStudio 5 thermocycling instrument (Applied BioSystems). Relative quantification was determined using the AACT method, where GAPDH was used as internal control and expression changes normalized to the reference sample (no siRNA treatment).

### In Vivo Mouse Study

### Animals

Male C57BL/6J mice (20-25 g) were group housed in the Saretius animal unit at the University of Reading, and maintained under a 12 h light/dark cycle, at 23° C. with humidity controlled according to Home Office regulations. Mice were given access to standard rodent chow SDS rat expanded diet (RM3-E-FG) for the duration of the study.

Formulation of siRNA Compounds

Compound A, Compound G, and Compound H were each formulated in RNAase free PBS to concentrations of 0.4 and 2 mg/mL, to provide doses of 2 and 10 mg/kg when given subcutaneously (SC) in a 5 mL/kg dosing volume. Control groups (n=5) received Vehicle (RNase-free PBS) SC at 5 mL/kg dosing volume.

### Liver Processing for RT-qPCR

At Day 2 (48 hrs) and Day 7 (168 hrs) following siRNA compound or Vehicle injection (n=5), each treatment group was terminally sampled by cardiac puncture under isoflurane. Liver tissue was excised and snap frozen in liquid N2. Total RNA was extracted from homogenates of snap-frozen whole liver using GenElute™ Total RNA Purification Kit (RNB100-100RXN)

Duplex RT-qPCR was performed using the ThermoFisher TaqMan Fast 1-Step Master Mix with TaqMan probes for GAPDH (VIC_PL), PCSK9 (FAM) and mTTR (FAM). Relative quantification (RQ) of PCSK9 was determined using the AACT method, where GAPDH was used as internal control and the expression changes of the target gene were normalized to the vehicle control.

### Duplex RT-qPCR Primary Mouse Hepatocytes

Cells were processed for RT-qPCR read-out using the Cells-to-CT 1-step TaqMan Kit 25 (Invitrogen™ A25603). Briefly, cells were washed with 50 μL ice-cold PBS and lysed in 20 μl Lysis solution containing DNase I. Lysis was stopped after 5 minutes by addition of 2 μl STOP Solution for 2 min. For the RT-qPCR analysis, 1 μL of lysate was dispensed per well into a 96-well PCR plate in a 10 μL RT-qPCR reaction volume. RT-qPCR was performed using the TaqMan® 1-Step qRT-PCR Mix from the Cells-to-CT 1-step TaqMan Kit, with TaqMan probes for GAPDH (VIC_PL, Assay Id Mm99999915_g1) or Complement C3 (FAM, Assay Id Mm01232779_ml). RT-qPCR was performed using a QuantStudio 5 thermocycling instrument (Applied BioSystems). Relative quantification was determined using the AACT method, where GAPDH was used as internal control and expression changes normalized to the reference sample (no siRNA treatment).

### Example 1

### Testing 5′ Versus 3′ Positioning of Crook on the Sense Strand (SS) of Unmodified ‘Inclisiran’ Sequence in Serum Stability Assays.

Following a 2 hr incubation in 10% FBS or 10% human serum, unmodified ‘Inclisiran’ with Crook positioned either at the 5′ or 3′ end of the SS, shows increased target mRNA (PCSK9) knockdown (KD) compared to the ‘no crook’ siRNA. However, superior KD is observed when crook is on the 5′ end compared to 3′ end of SS, following pre-treatment in human serum.

This is demonstrated in FIG. 1, where 5′ SS crook siRNA [striped bar] containing hairpin sequence GCGAAGC, maintains high levels of target KD (85%) in HepG2 cells following 2 hr treatment with 10% human serum comparable to that observed with modified Inclisiran (80%) [white bar]. Similar results can be shown when ‘reversed’ crook hairpin (CGAAGCG) is placed at the 5′ end of the SS [spotted bar]. In contrast, 3′ SS positioned Crook [hatched bar] shows-18.75% (loss of target KD) in HepG2 cells following pre-treatment in human serum; 65% KD (compared to 80% KD with no serum incubation). As expected, unmodified ‘Inclisiran’ with no Crook attached [grey bar] shows reduced levels of target KD after pre-treatment in either FBS or human serum: 50% and 60% KD, respectively, equating to −26.8% and −39% loss of KD.

### Example 2

Testing 5′ Positioning of Crook on the Sense Strand (SS) of Unmodified ‘Inclisiran’ Sequence in Serum Stability Assays with Increasing Concentrations of FBS.

Following 2 hr incubations at 37 C in increasing concentrations of FBS, unmodified ‘Inclisiran’ sequence with Crook positioned at the 5′ end of the SS [striped bar] shows sustained target mRNA (PCSK9) knockdown (70-80% KD) in all concentrations of FBS tested (10%, 20% and 50%), comparable to levels observed with modified Inclisiran (70-80% KD) [white bar]. Similarly, ‘reversed’ crook hairpin (CGAAGCG) on the 5′ end of SS provides 65-75% KD with no loss of KD [spotted bar]. In contrast, the ‘no crook’ compound [grey bar] displays up to −85% loss of KD as only 20-50% target KD, is evident following serum treatment.

### Example 3

Testing 5’ Positioning of Crook on the Sense Strand (SS) of Unmodified ‘Inclisiran’

sequence in serum stability assays over a 4-hr incubation period.

After a 4 hr incubation in either 10% FBS or 10% human serum, unmodified ‘Inclisiran’ with Crook positioned at the 5′ end of the SS, shows sustained levels of approx. 75% target mRNA (PCSK9) knockdown (KD), and 65% KD, respectively [striped bar]. Similarly, there is no loss of KD evident for ‘reversed’ crook hairpin (CGAAGCG) on the 5′ end of SS [spotted bar], comparable with modified ‘Inclisiran’, where approx . . . 70% KD is observed [white bar]. In contrast, the absence of Crook [grey bar] leads to subtantially lower levels of KD following 4 hr pre-treatment in 10% FBS (45% KD) or 10% human serum (35% KD), equating to a −36% and −50% loss in KD, respectively.

### Example 4

Testing 5′ Versus 3′ Positioning of Crook on an Unmodified siRNA Sequence Targeting PCSK9, in Serum Stability Assays (Sequence Termed PC8-18).

Following a 2 hr incubation in 10% FBS or 10% human serum, PC8-18 with Crook positioned at the 5′ end of the sense strand (SS), shows superior levels of knockdown (KD) of target mRNA (PCSK9) compared to 3′ positioned Crook on either the SS or AS. This is shown in FIG. 4, where there is sustained target KD (approx. 85%) for PC8-18 siRNA with 5′SS Crook: [horizontal striped bar & spots on black background bar] compared to 60-70% KD (equating to a loss of 30% KD compared to no serum treatment) seen with 3′ SS positioned Crook [spots on white background bar & hatched bar]. Similarly, when Crook is placed on 3′ AS, loss of KD is 6-16% resulting in 65-75% target KD [vertical striped bar]. When Crook is not present on PC8-18 siRNA, target KD is reduced to only 35% following 2 hr incubation in FBS equating to a substantial loss of KD (−63% compared to no serum treatment), and to only 25% KD in human serum (−77%). Similarly, uncrooked molecules that contain 3′ dTdT overhangs, show loss of KD levels of −44% and −72% (compared to no serum treatment) following pre-treatment in FBS and human serum, respectively.

### Example 5 Testing the In Vivo Silencing Effect of 5′ Versus 3′ Positioning of Crook on an Unmodified siRNA Compounds Targeting PCSK9 (PC2 Sequence)

Groups of 5 mice for each treatment group were injected subcutaneously (SC) with either vehicle (PBS), compound A (no Crook), compound G (Crook on 5′ end of sense strand (SS)), or compound H (Crook on 3′ end of SS). Each compound was given at either 2 mg/kg or 10 mg/kg, and following sacrifice, levels of liver PCSK9 mRNA were measured at two time points (day 2 and day 7).

Compound G (5′ SS Crook) results in 40% KD of PCSK9 mRNA in the liver after 48 hours at 2 and 10 mg/kg and 30% KD at 10 mg/kg after 7 days, compared to vehicle controls (FIG. 5A). Comparable liver target KD is seen after 48 hrs for compound H (3′ SS Crook) approx. 50% KD at 2 mg/kg (30% KD at 10 mg/kg), with no significant KD observed at day 7 (FIG. 5A). Compound A which contains no Crook, shows noticeably less target KD, with no silencing following SC injection of 2 mg/kg dose at either 2 or 7 days. At the 10 mg/kg dose, compound A shows and <20% KD after 48 hrs, and 40% after 7 days (FIG. 5A).

### Example 6

### Testing Compounds a, G and H in Serum Stability Assays (HepG2 Cells).

Comparable results are shown for both 5′ and 3′ positioned Crook on the SS. Compound G (5′ SS Crook) and compound H (3′ SS Crook) maintains PCSK9 mRNA KD of >50% following a 2 hr incubation in either 10% FBS or human serum (compared to no serum treatment). In contrast, there is loss of target KD seen for compound A (no Crook), from 50% to only 20% KD following a 2 hr serum treatment; FIG. 5B.

When these siRNA compounds were further challenged in increasing serum concentrations (20% and 50%) over a 2 hr period, compound G (5′SS Crook) displayed superior performance over 3′SS positioned Crook (H) in human serum. This is shown in FIG. 5C, where a sustained level of target mRNA KD (approx. 50%) is evident only in compound G [spotted bar] following 2 hrs incubation in 50% human serum. This equates to no loss of KD for G when compared to its ‘no serum’ treatment KD level. In contrast, compound H [grey bar] shows a complete loss in KD (0%) performing exactly as ‘no crook’ compound A [white bar] after 2 hrs in 50% human serum.

### Example 7

### C3 Silencing in Primary Mouse Hepatocytes

As shown in Table 1, Complement C3 siRNAs showed high level of knockdown (KD) of target mRNA when transfected in primary mouse hepatocytes at a concentration of 2.5 and 25 nM. In particular, the majority of the sequences (85.4%) showed KD levels ≥90% at 2.5 nM while almost all the siRNAs tested (97.5%) showed KD levels ≥90% at 25 nM. When pre-incubated in 50% human serum for 2 hours followed by transfection in primary mouse hepatocytes for 48 hours at 25 nM, 27 siRNAs out of 41 (66%) remained stable in serum showing loss of KD below 15% when compared to same siRNA pre-incubated in vehicle (Table 1).

TABLE 1
Efficacy and serum stability of Complement C3 siRNAs

| | KD levels (%) at 2.5 and 25 nM and | | | |
|---|---|---|---|---|
| | stability in 50% human serum | | | |
| siRNA | 2.5 nM + | 25 nM + | 25 nM + | % loss of KD |
| ID | vehicle | vehicle | 50% HS | in serum |
| S01-OR | 86.4 | 88.3 | 86.7 | 1.6 |
| CC3_1 | 96.4 | 96.3 | 38.5 | 57.8 |
| CC3_2 | 94.2 | 93.6 | 95.6 | 0 |
| CC3_3 | 91.8 | 94.6 | 64.6 | 30.0 |
| CC3_4 | 94.0 | 93.9 | 95.9 | 0 |
| CC3_5 | 90.1 | 94.8 | 75.5 | 19.3 |
| CC3_6 | 97.0 | 97.5 | 95.2 | 2.2 |
| CC3_7 | 96.7 | 96.2 | 97.2 | 0 |
| CC3_8 | 97.3 | 98.0 | 98.5 | 0 |
| CC3_9 | 92.9 | 95.8 | 64.3 | 31.4 |
| CC3_10 | 94.8 | 96.9 | 73.2 | 23.7 |
| CC3_11 | 95.7 | 98.6 | 99.0 | 0 |
| CC3_12 | 94.4 | 96.7 | 96.9 | 0 |
| CC3_13 | 96.0 | 96.0 | 70.2 | 25.7 |
| CC3_14 | 94.0 | 95.8 | 61.9 | 33.9 |
| CC3_15 | 96.0 | 97.1 | 89.3 | 7.8 |
| CC3_16 | 93.1 | 95.5 | 96.7 | 0 |
| CC3_17 | 83.9 | 90.8 | 86.8 | 4.0 |
| CC3_18 | 88.6 | 96.9 | 82.5 | 14.3 |
| CC3_19 | 91.5 | 97.0 | 53.5 | 43.5 |
| CC3_20 | 95.2 | 97.5 | 26.1 | 71.4 |
| CC3_21 | 96.0 | 96.5 | 70.5 | 26.1 |
| CC3_22 | 91.5 | 94.2 | 90.8 | 3.4 |
| CC3_23 | 91.5 | 93.0 | 92.5 | 0.5 |
| CC3_24 | 90.2 | 95.2 | 93.3 | 1.8 |
| CC3_25 | 89.2 | 95.0 | 89.7 | 5.3 |
| CC3_26 | 95.2 | 97.6 | 94.2 | 3.4 |
| CC3_27 | 97.8 | 97.4 | 96.4 | 1.0 |
| CC3_28 | 97.8 | 97.4 | 58.9 | 38.5 |
| CC3_29 | 97.5 | 97.6 | 90.0 | 7.6 |
| CC3_30 | 96.2 | 97.2 | 42.0 | 55.2 |
| CC3_31 | 97.4 | 98.3 | 97.6 | 0.8 |
| CC3_32 | 95.6 | 96.4 | 82.2 | 14.2 |
| CC3_33 | 95.0 | 97.4 | 41.0 | 56.4 |
| CC3_34 | 95.9 | 96.7 | 94.1 | 2.6 |
| CC3_35 | 95.4 | 97.7 | 96.6 | 1.1 |
| CC3_36 | 92.4 | 96.1 | 95.1 | 1.1 |
| CC3_37 | 82.5 | 90.4 | 91.6 | 0.0 |
| CC3_38 | 89.8 | 95.4 | 95.1 | 0.3 |
| CC3_39 | 94.4 | 96.9 | 96.5 | 0.4 |
| CC3_40 | 95.5 | 98.0 | 35.2 | 62.8 |

Selection of siRNA Sequences that Optimally Silence Complement Component 3, Complement Component 5 and MASP-2

TABLE 2
C3 selection

| SEQ | | SEQ | |
|---|---|---|---|
| ID | | ID | |
| NO | Sense Sequence | NO | Antisense Sequence |
| 61 | GCCCUUUGACCUCAUGGU | 11 | AACACCAUGAGGUCAAAGGGC |
| | GUU | | |
| 2 | UGCCCUUUGACCUCAUGG | 12 | ACACCAUGAGGUCAAAGGGCA |
| | UGU | | |
| 3 | CCUCUUCAUCCAGACAGA | 13 | UUGUCUGUCUGGAUGAAGAGG |
| | CAA | | |
| 4 | CCCUUUGACCUCAUGGUG | 14 | GAACACCAUGAGGUCAAAGGG |
| | UUC | | |
| 5 | ACCUCUUCAUCCAGACAG | 15 | UGUCUGUCUGGAUGAAGAGGU |
| | ACA | | |
| 6 | GCGGGUACCUCUUCAUCC | 16 | UCUGGAUGAAGAGGUACCCGC |
| | AGA | | |
| 7 | GACAGACAAGACCAUCUA | 17 | GUGUAGAUGGUCUUGUCUGUC |
| | CAC | | |
| 8 | UCCAGACAGACAAGACCA | 18 | AGAUGGUCUUGUCUGUCUGGA |
| | UCU | | |
| 9 | CUUCAUCCAGACAGACAA | 19 | GUCUUGUCUGUCUGGAUGAAG |
| | GAC | | |
| 10 | CAUCCAGACAGACAAGAC | 20 | AUGGUCUUGUCUGUCUGGAUG |
| | CAU | | |

TABLE 3
C5 selection

| SEQ | | SEQ | |
|---|---|---|---|
| ID | | ID | |
| NO | Sense Sequence | NO | Antisense Sequence |
| 21 | AAGGAACUGUUUACAACUA | 31 | UAUAGUUGUAAACAGUUCCU |
| | UA | | U |
| 22 | CUGGUAUAUGUGUUGCUGA | 32 | UAUCAGCAACACAUAUACCAG |
| | UA | | |
| 23 | CUUUUCCUGACUUCAAGAU | 33 | GAAUCUUGAAGUCAGGAAAA |
| | UC | | G |
| 24 | UUGAAAGGAACUGUUUACA | 34 | GUUGUAAACAGUUCCUUUCA |
| | AC | | A |
| 25 | AUUGAAAGGAACUGUUUAC | 35 | UUGUAAACAGUUCCUUUCAA |
| | AA | | U |
| 26 | UUUUCCUGACUUCAAGAUU | 36 | GGAAUCUUGAAGUCAGGAAA |
| | CC | | A |
| 27 | AACUGUCUUAACUUUCAUA | 37 | UCUAUGAAAGUUAAGACAGU |
| | GA | | U |
| 28 | UAUCUCUUUUCCUGACUUC | 38 | UUGAAGUCAGGAAAAGAGAU |
| | AA | | A |
| 29 | ACUGGUAUAUGUGUUGCUG | 39 | AUCAGCAACACAUAUACCAGU |
| | AU | | |
| 30 | UCUUUUCCUGACUUCAAGA | 40 | AAUCUUGAAGUCAGGAAAAG |
| | UU | | A |

TABLE 4
MASP- 2 selection

| SEQ | | SEQ | |
|---|---|---|---|
| ID | | ID | |
| NO | Sense Sequence | NO | Antisense Sequence |
| 41 | ACCUACAAAGCUGUGAUU | 51 | CUGAAUCACAGCUUUGUA |
| | CAG | | GGU |
| 42 | UGUGAUUCAGUACAGCUG | 52 | UCACAGCUGUACUGAAUC |
| | UGA | | ACA |
| 43 | GUGGUUUGUGGGAGGAAU | 53 | ACUAUUCCUCCCACAAACC |
| | AGU | | AC |
| 44 | GCGCCUCUACUUCACCCAC | 54 | AAGUGGGUGAAGUAGAGG |
| | UU | | CGC |
| 45 | UGUGGAGUCCUUCGAUGU | 55 | UCCACAUCGAAGGACUCC |
| | GGA | | ACA |
| 46 | GAUGGUAAAUAUGUGUGU | 56 | CUCACACACAUAUUUACC |
| | GAG | | AUC |
| 47 | CCUACAAAGCUGUGAUUC | 57 | ACUGAAUCACAGCUUUGU |
| | AGU | | AGG |
| 48 | AUUCAGUACAGCUGUGAA | 58 | CUCUUCACAGCUGUACUG |
| | GAG | | AAU |
| 49 | UGGAGUCCUUCGAUGUGG | 59 | UCUCCACAUCGAAGGACU |
| | AGA | | CCA |
| 50 | CUGUGAUUCAGUACAGCU | 60 | CACAGCUGUACUGAAUCA |
| | GUG | | CAG |

TABLE 5
C3/C5 selection

| | | |
|---|---|---|
| 69 | COMPC3_01; | 5′-UCUUGGUGAAGUGGAUCUG |
| 70 | COMPC3_02; | 5′-UGAGAGAAGACCUUGACCA |
| 71 | COMPC3_03; | 5′-AGAGAGAAGACCUUGACCA |
| 72 | COMPC3_04; | 5′-GUUGUAAUAGGCGUAGACC |
| 73 | COMPC3_05; | 5′-AUUGUAAUAGGCGUAGACC |
| 74 | COMPC3_06; | 5′-CUCUACGAAGCUCAUGAAUAU |
| 75 | COMPC3_07; | 5′-CUGCAGGAGGCUAAAGAUAUU |
| 76 | COMPC3_08; | 5′-CUCCACUGAGUUUGAGGUGAA |
| 77 | COMPC3_09; | 5′-GUCCAAUGACUUUGACGAGUA |
| 78 | COMPC3_10; | 5′-CAGGAGUAACCUGGAUGAGGA |
| 79 | COMPC3_01; | 5′-UCUUGGUGAAGUGGAUCUG |
| 80 | COMPC3_02; | 5′-UGAGAGAAGACCUUGACCA |
| 81 | COMPC3_03; | 5′-AGAGAGAAGACCUUGACCA |
| 82 | COMPC3_04; | 5′-GUUGUAAUAGGCGUAGACC |
| 83 | COMPC3_05; | 5′-AUUGUAAUAGGCGUAGACC |
| 84 | COMPC3_06; | 5′-CUCUACGAAGCUCAUGAAUAU |
| 85 | COMPC3_07; | 5′-CUGCAGGAGGCUAAAGAUAUU |
| 86 | COMPC3_08; | 5′-CUCCACUGAGUUUGAGGUGAA |
| 87 | COMPC3_09; | 5′-GUCCAAUGACUUUGACGAGUA |
| 88 | COMPC3_10; | 5′-CAGGAGUAACCUGGAUGAGGA |
| 89 | COMPC3_01; | 5′-CAGAUCCACUUCACCAAGA-3′ |
| 90 | COMPC3_02; | 5′-UGGUCAAGGUCUUCUCUCU-3′ |
| 91 | COMPC3_03; | 5′-UGGUCAAGGUCUUCUCUCU-3′ |
| 92 | COMPC3_04; | 5′-GGUCUACGCCUAUUACAAC-3′ |
| 93 | COMPC3_05; | 5′-GGUCUACGCCUAUUACAAU-3′ |
| 94 | COMPC3_06; | 5′-AUAUUCAUGAGCUUCGUAGAG-3′ |
| 95 | COMPC3_07; | 5′-AAUAUCUUUAGCCUCCUGCAG-3′ |
| 96 | COMPC3_08; | 5′-UUCACCUCAAACUCAGUGGAG-3′ |
| 97 | COMPC3_09; | 5′-UACUCGUCAAAGUCAUUGGAC-3′ |
| 98 | COMPC3_10; | 5′-UCCUCAUCCAGGUUACUCCUG-3′ |
| 99 | COMPC3_01; | 5′-CAGAUCCACUUCACCAAGA |
| 100 | COMPC3_02; | 5′-UGGUCAAGGUCUUCUCUCU |
| 101 | COMPC3_03; | 5′-UGGUCAAGGUCUUCUCUCU |
| 102 | COMPC3_04; | 5′-GGUCUACGCCUAUUACAAC |
| 103 | COMPC3_05; | 5′-GGUCUACGCCUAUUACAAU |
| 104 | COMPC3_06; | 5′-AUAUUCAUGAGCUUCGUAGAG |
| 105 | COMPC3_07; | 5′-AAUAUCUUUAGCCUCCUGCAG |
| 106 | COMPC3_08; | 5′-UUCACCUCAAACUCAGUGGAG |
| 107 | COMPC3_09; | 5′-UACUCGUCAAAGUCAUUGGAC |
| 108 | COMPC3_10; | 5′-UCCUCAUCCAGGUUACUCCUGtc |
| 109 | COMPC5_01; | 5′-AAGCAAGAUAUUUUUAUAAUA |
| 110 | COMPC5_02; | 5′-GACGAUCAAGGCUAAAUAUAA |
| 111 | COMPC5_03; | 5′-UCCCAUCAAGGUGCAGGUUAA |
| 112 | COMPC5_04; | 5′-CAGUCUGAACUUGAAAGAUAU |
| 113 | COMPC5_05; | 5′-UUCAUUUAUCCUCAGAGAAUA |
| 114 | COMPC5_06; | 5′-GAGCAUUAUGUCCUACAGAAA |
| 115 | COMPC5_07; | 5′-GACUGAUAACCAUAAGGCUUU |
| 116 | COMPC5_08; | 5′-CUGUCUUAACUUUCAUAGAUC |
| 117 | COMPC5_09; | 5′-AAUGAUGAACCUUGUAAAGAA |
| 118 | COMPC5_10; | 5′-CUCAAUCGAGCCAGAAUAUAA |
| 119 | COMPC5_01; | 5′-AAGCAAGAUAUUUUUAUAAUA |
| 120 | COMPC5_02; | 5′-GACGAUCAAGGCUAAAUAUAA |
| 121 | COMPC5_03; | 5′-UCCCAUCAAGGUGCAGGUUAA |
| 122 | COMPC5_04; | 5′-CAGUCUGAACUUGAAAGAUAU |
| 123 | COMPC5_05; | 5′-UUCAUUUAUCCUCAGAGAAUA |
| 124 | COMPC5_06; | 5′-GAGCAUUAUGUCCUACAGAAA |
| 125 | COMPC5_07; | 5′-GACUGAUAACCAUAAGGCUUU |
| 126 | COMPC5_08; | 5′-CUGUCUUAACUUUCAUAGAUC |
| 127 | COMPC5_09; | 5′-AAUGAUGAACCUUGUAAAGAA |
| 128 | COMPC5_10; | 5′-CUCAAUCGAGCCAGAAUAUAA |
| 129 | COMPC5_01; | 5′-UAUUAUAAAAAUAUCUUGCUUUU-3′ |
| 130 | COMPC5_02; | 5′-UUAUAUUUAGCCUUGAUCGUC-3′ |
| 131 | COMPC5_03; | 5′-UUAACCUGCACCUUGAUGGGA-3′ |
| 132 | COMPC5_04; | 5′-AUAUCUUUCAAGUUCAGACUG-3′ |
| 133 | COMPC5_05; | 5′-UAUUCUCUGAGGAUAAAUGAA-3′ |
| 134 | COMPC5_06; | 5′-UUUCUGUAGGACAUAAUGCUC-3′ |
| 135 | COMPC5_07; | 5′-AAAGCCUUAUGGUUAUCAGUC-3′ |
| 136 | COMPC5_08; | 5′-GAUCUAUGAAAGUUAAGACAG-3′ |
| 137 | COMPC5_09; | 5′-UUCUUUACAAGGUUCAUCAUU-3′ |
| 138 | COMPC5_10; | 5′-UUAUAUUCUGGCUCGAUUGAG-3′ |
| 139 | COMPC5_01; | 5′-UAUUAUAAAAAUAUCUUGCUUUU |
| 140 | COMPC5_02; | 5′-UUAUAUUUAGCCUUGAUCGUC |
| 141 | COMPC5_03; | 5′-UUAACCUGCACCUUGAUGGGA |
| 142 | COMPC5_04; | 5′-AUAUCUUUCAAGUUCAGACUG |
| 143 | COMPC5_05; | 5′-UAUUCUCUGAGGAUAAAUGAA |
| 144 | COMPC5_06; | 5′-UUUCUGUAGGACAUAAUGCUC |
| 145 | COMPC5_07; | 5′-AAAGCCUUAUGGUUAUCAGUC |
| 146 | COMPC5_08; | 5′-GAUCUAUGAAAGUUAAGACAG |
| 147 | COMPC5_09; | 5′-UUCUUUACAAGGUUCAUCAUU |
| 148 | COMPC5_10; | 5′-UUAUAUUCUGGCUCGAUUGAG |

TABLE 6
siRNAs pairs used in silencing of C3 and C5 gene expression in
HEP2G cells in vitro

| | | |
|---|---|---|
| COMPC3_01 | 5′-UCUUGGUGAAGUGGAUCUG-3′ | 5′-CAGAUCCACUUCACCAAGA-3′ |
| (SEQ ID | | |
| NO 69 | | |
| and 89) | | |
| COMPC3_02 | 5′-UGAGAGAAGACCUUGACCA-3′ | 5′-UGGUCAAGGUCUUCUCUCU-3′ |
| (SEQ ID | | |
| ID NO 70 | | |
| and 90) | | |
| COMPC3_03 | 5′-AGAGAGAAGACCUUGACCA-3′ | 5′-UGGUCAAGGUCUUCUCUCU-3′ |
| (SEQ ID | | |
| NO 71 | | |
| and 91) | | |
| COMPC3_04 | 5′-GUUGUAAUAGGCGUAGACC-3′ | 5′-GGUCUACGCCUAUUACAAC-3′ |
| (SEQ ID | | |
| ID NO 72 | | |
| and 92) | | |
| COMPC3_05 | 5′-AUUGUAAUAGGCGUAGACC-3′ | 5′-GGUCUACGCCUAUUACAAU-3′ |
| (SEQ ID | | |
| NO 73 | | |
| and 93) | | |
| COMPC3_06 | 5′-CUCUACGAAGCUCAUGAAUAU-3′ | 5′-AUAUUCAUGAGCUUCGUAGAG-3′ |
| (SEQ ID | | |
| NO 74 | | |
| and 94) | | |
| COMPC3_07 | 5′-CUGCAGGAGGCUAAAGAUAUU-3′ | 5′-AAUAUCUUUAGCCUCCUGCAG-3′ |
| (SEQ ID | | |
| NO 75 | | |
| and 95) | | |
| COMPC3_08 | 5′-CUCCACUGAGUUUGAGGUGAA-3′ | 5′-UUCACCUCAAACUCAGUGGAG-3′ |
| (SEQ ID | | |
| NO 76 | | |
| and 96) | | |
| COMPC3_09 | 5′-GUCCAAUGACUUUGACGAGUA-3′ | 5′-UACUCGUCAAAGUCAUUGGAC-3′ |
| (SEQ ID | | |
| NO 77 | | |
| and 97) | | |
| COMPC3_10 | 5′-CAGGAGUAACCUGGAUGAGGA-3′ | 5′-UCCUCAUCCAGGUUACUCCUG-3′ |
| (SEQ ID | | |
| NO 78 | | |
| and 98) | | |
| COMPC5_01 | 5′-AAGCAAGAUAUUUUUAUAAUA-3′ | 5′-UAUUAUAAAAAUAUCUUGCU |
| (SEQ ID | | UUUtt-3′ |
| NO 109 | | |
| and 129) | | |
| COMPC5_02 | 5′-GACGAUCAAGGCUAAAUAUAA-3′ | 5′-UUAUAUUUAGCCUUGAUCGUC-3′ |
| (SEQ ID | | |
| NO 110 | | |
| and 130) | | |
| COMPC5_03 | 5′-UCCCAUCAAGGUGCAGGUUAA-3′ | 5′-UUAACCUGCACCUUGAUGGGA-3′ |
| (SEQ ID | | |
| NO 111 | | |
| and 131) | | |
| COMPC5_04 | 5′-CAGUCUGAACUUGAAAGAUAU-3′ | 5′-AUAUCUUUCAAGUUCAGACUG-3′ |
| (SEQ ID | | |
| NO 112 | | |
| and 132) | | |
| COMPC5_05 | 5′-UUCAUUUAUCCUCAGAGAAUA-3′ | 5′-UAUUCUCUGAGGAUAAAUGAA-3′ |
| (SEQ ID | | |
| NO 113 | | |
| and 133) | | |
| COMPC5_06 | 5′-GAGCAUUAUGUCCUACAGAAA-3′ | 5′-UUUCUGUAGGACAUAAUGCUC-3′ |
| (SEQ ID | | |
| NO 114 | | |
| and 134) | | |
| COMPC5_07 | 5′-GACUGAUAACCAUAAGGCUUU-3′ | 5′-AAAGCCUUAUGGUUAUCAGUC-3′ |
| (SEQ ID | | |
| NO 115 | | |
| and 135) | | |
| COMPC5_08 | 5′-CUGUCUUAACUUUCAUAGAUC-3′ | 5′-GAUCUAUGAAAGUUAAGACAG-3′ |
| (SEQ ID | | |
| NO 116 | | |
| and 136) | | |
| COMPC5_09 | 5′-AAUGAUGAACCUUGUAAAGAA-3′ | 5′-UUCUUUACAAGGUUCAUCAUU-3′ |
| (SEQ ID | | |
| NO 117 | | |
| and 137) | | |
| COMPC5_10 | 5′-CUCAAUCGAGCCAGAAUAUAA-3′ | 5′-UUAUAUUCUGGCUCGAUUGAG-3′ |
| (SEQ ID | | |
| NO 118 | | |
| and 138) | | |

TABLE 7
Crook structures tested in the serum stability assay for 5′ crook
siRNAs 14b to siRNA15-5′CR consist of unmodified ′inclisiran′ sequence
(C = crook; CR = reversed hairpin Crook).
siRNAs 35-44 consist of PC8 sequence
siRNAs A, G and H consist of PC2 sequence

| Oligo name | Sequence |
|---|---|
| siRNA14m | Sense: 5′ Cm*Um* Am Gm Am Cm Cf Um Gf Um t Um Um Gm Cm |
| | Um Um Um Um Gm Um 3′ |
| ‘Inclisiran’ | Antisense: 5′ Am*Cf*Am Af Af Af Gm Cf Am Af Am Af Cm Af Gm |
| | Gf Um Cf Um Am Gm* Am* Am 3′ |
| siRNA14b | Sense (5′-3′): CUAGACCUGUtUUGCUUUUGU |
| | Antisense (5′-3′): ACAAAAGCAAAACAGGUCUAGAA (SEQ ID NO 1192) |
| siRNA15b | Sense (5′-3′): CUAGACCUGUtUUGCUUUUGUtcacctcatcccgcgaagc |
| | Antisense (5′-3′): ACAAAAGCAAAACAGGUCUAGAA (SEQ ID NO 1192) |
| siRNA15-5’C | Sense (5′-3′): cgaagcgccctactccactCUAGACCUGUtUUGCUUUUGU |
| | Antisense (5′-3′): ACAAAAGCAAAACAGGUCUAGAA (SEQ ID NO 1192) |
| siRNA15-5’CR | 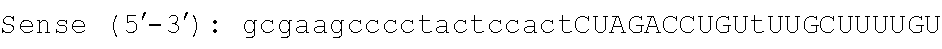 |
| | Antisense (5′-3′): ACAAAAGCAAAACAGGUCUAGAA (SEQ ID NO 1192) |
| siRNA35 | Sense (5′-3′): CAGGUCUGGAAUGCAAAGUCA (SEQ ID NO 1193) |
| | Antisense (5′-3′): UGACUUUGCAUUCCAGACCUG (SEQ ID NO 1194) |
| siRNA36 | Sense (5′-3′): CAGGUCUGGAAUGCAAAGUCAdTdT |
| | Antisense (5′-3′): UGACUUUGCAUUCCAGACCUGdTdT |
| siRNA37 | Sense (5′-3′): CAGGUCUGGAAUGCAAAGUCAdTdCdAdCdCdTdCdAdTdCdCdCdGdCdGdAdAdGdC |
| | Antisense (5′-3′): UGACUUUGCAUUCCAGACCUG (SEQ ID NO 1194) |
| siRNA 38 | Sense (5′-3′): CAGGUCUGGAAUGCAAAGUCA (SEQ ID NO 1193) |
| | Antisense (5′-3′): UGACUUUGCAUUCCAGACCUGdTdCdAdCdCdTdCdAdTdCdCdCdGdCdGdAdAdGdC |
| siRNA39 | Sense (5′-3′): CAGGUCUGGAAUGCAAAGUCAdTdCdAdCdCdTdCdAdTdCdCdCdGdCdGdAdAdGdC |
| | Antisense (5′-3′): UGACUUUGCAUUCCAGACCUGdTdT |
| siRNA40 | Sense (5′-3′): CAGGUCUGGAAUGCAAAGUCAdTdT |
| | Antisense (5′-3′): UGACUUUGCAUUCCAGACCUGdTdCdAdCdCdTdCdAdTdCdCdCdGdCdGdAdAdGdC |
| siRNA41 | Sense (5′-3′): dCdGdAdAdGdCdGdCdCdCdTdAdCdTdCdCdAdCdTCAGGUCUGGAAUGCAAAGUCA |
| | Antisense (5′-3′): UGACUUUGCAUUCCAGACCUG (SEQ ID NO 1194) |
| siRNA42 | Sense (5′-3′): dCdGdAdAdGdCdGdCdCdCdTdAdCdTdCdCdAdCdTCAGGUCUGGAAUGCAAAGUCA |
| | Antisense (5′-3′): UGACUUUGCAUUCCAGACCUGdTdT |
| siRNA43 | Sense (5′-3′): dCdGdAdAdGdCdGdCdCdCdTdAdCdTdCdCdAdCdTCAGGUCUGGAAUGCAAAGUCAdTdT |
| | Antisense (5′-3′): UGACUUUGCAUUCCAGACCUG (SEQ ID NO 1194) |
| siRNA44 | Sense (5′-3′): CAGGUCUGGAAUGCAAAGUCA (SEQ ID NO 1193) |
| | Antisense (5′-3′): dCdGdAdAdGdCdGdCdCdCdTdAdCdTdCdCdAdCdTUGACUUUGCAUUCCAGACCUG |
| siRNA-A | 5′-AGGCCUGGAGUUUAUUCGGAA GalNAc-3′ |
| | 3′-ttUCCGGACCUCAAAUAAGCCUU-5′ |
| siRNA-G | 5′-cgaagcgccctactccactA*G*GCCUGGAGUUUAUUCGGAA |
| | GalNAc-3′ |
| | 3′-t*t*UCCGGACCUCAAAUAAGCC*U*U-5′ |
| siRNA-H | 5′-AGGCCUGGAGUUUAUUCGGAAtcacctcatcccgcgaagc-3′ |
| | 3′-GalNAc UCCGGACCUCAAAUAAGCCUU-5′ |

Legend:
c, g, a, t or dT, dG, dA, dC: DNA bases
A, G, C, U: RNA bases
f: 2′-deoxy-2′-fluoro
m: 2′-O-methyl
* internucleotide linkage phosphorothioate (PS)
GalNAc

TABLE 8
Complement C3 siRNA sequences

| siRNA ID | Sense (5′-3′) | Antisense (5′- 3′) |
|---|---|---|
| CC3_01; SEQ ID | AGACAUAAUUCCAGAAGAA | UUCUUCUGGAAUUAUGUCUUU |
| NO 1195 and 1196 | | |
| CC3_02; SEQ ID | GGGAAACGGUGGUGGAGAA | UUCUCCACCACCGUUUCCCUU |
| NO 1197 and 1198 | | |
| CC3_03; SEQ ID | CCGCAGAGUUUGAGGUGAA | UUCACCUCAAACUCUGCGGUU |
| NO 1199 and 1200 | | |
| CC3_04; SEQ ID | GUUGAAAGAACCAGAGAAA | UUUCUCUGGUUCUUUCAACUU |
| NO 1201 and 1202 | | |
| CC3_05; SEQ ID | AGUUUGAGGUGAAGGAAUA | UAUUCCUUCACCUCAAACUUU |
| NO 1203 and 1204 | | |
| CC3_06; SEQ ID | GGGUGGAACUGUUGCAUAA | UUAUGCAACAGUUCCACCCUU |
| NO 1205 and 1206 | | |
| CC3_07; SEQ ID | GGACAAAGCUGGUCAGUAC | GUACUGACCAGCUUUGUCCUU |
| NO 1207 and 1208 | | |
| CC3_08; SEQ ID | GGAGUGAAUUGGAGGAAGA | UCUUCCUCCAAUUCACUCCUU |
| NO 1209 and 1210 | | |
| CC3_09; SEQ ID | CCAUAGAAGAGUUGAAAGA | UCUUUCAACUCUUCUAUGGUU |
| NO 1211 and 1212 | | |
| CC3_10; SEQ ID | ACAUAGAGCUGUUGGAUGA | UCAUCCAACAGCUCUAUGUUU |
| NO 1213 and 1214 | | |
| CC3_11; SEQ ID | CGGAAGAGACCAAGCAAAA | UUUUGCUUGGUCUCUUCCGUU |
| NO 1215 and 1216 | | |
| CC3_12; SEQ ID | GGAAAGAAGGAUGGACAAA | UUUGUCCAUCCUUCUUUCCUU |
| NO 1217 and 1218 | | |
| CC3_13; SEQ ID | GCAUAAAGGCCUUCAUAGA | UCUAUGAAGGCCUUUAUGCUU |
| NO 1219 and 1220 | | |
| CC3_14; SEQ ID | CUACGAAGGUCAUGAACAU | AUGUUCAUGACCUUCGUAGUU |
| NO 1221 and 1222 | | |
| CC3_15; SEQ ID | CCUCUGGAGUAGAUAGAUA | UAUCUAUCUACUCCAGAGGUU |
| NO 1223 and 1224 | | |
| CC3_16; SEQ ID | GAGAAAGCAGUGAUGGUAA | UUACCAUCACUGCUUUCUCUU |
| NO 1225 and 1226 | | |
| CC3_17; SEQ ID | GAGGAUGGUAUGCGGGAUA | UAUCCCGCAUACCAUCCUCUU |
| NO 1227 and 1228 | | |
| CC3_18; SEQ ID | CCAGAAGGAAUGAGAAUCA | UGAUUCUCAUUCCUUCUGGUU |
| NO 1229 and 1230 | | |
| CC3_19; SEQ ID | CCAAGUACGAGAUGAACAA | UUGUUCAUCUCGUACUUGGUU |
| NO 1231 and 1232 | | |
| CC3_20; SEQ ID | CAGAAUCUAUGGUGGUUUA | AAACCACCAUAGAUUCUGUU |
| NO 1233 and 1234 | | |
| CC3_21; SEQ ID | GAAGACAUAAUUCCAGAAGAA | UUCUUCUGGAAUUAUGUCUUCUU |
| NO 1235 and 1236 | | |
| CC3_22; SEQ ID | CGGGGAAACGGUGGUGGAGAA | UUCUCCACCACCGUUUCCCCGUU |
| NO 1237 and 1238 | | |
| CC3_23; SEQ ID | CUCCGCAGAGUUUGAGGUGAA | UUCACCUCAAACUCUGCGGAGUU |
| NO 1239 and 1240 | | |
| CC3_24; SEQ ID | GAGUUGAAAGAACCAGAGAAA | UUUCUCUGGUUCUUUCAACUCUU |
| NO 1241 and 1242 | | |
| CC3_25; SEQ ID | AGAGUUUGAGGUGAAGGAAUA | UAUUCCUUCACCUCAAACUCUUU |
| NO 1243 and 1244 | | |
| CC3_26; SEQ ID | GAGGGUGGAACUGUUGCAUAA | UUAUGCAACAGUUCCACCCUCUU |
| NO 1245 and 1246 | | |
| CC3_27; SEQ ID | AUGGACAAAGCUGGUCAGUAC | GUACUGACCAGCUUUGUCCAUUU |
| NO 1247 and 1248 | | |
| CC3_28; SEQ ID | CAGGAGUGAAUUGGAGGAAGA | UCUUCCUCCAAUUCACUCCUGUU |
| NO 1249 and 1250 | | |
| CC3_29; SEQ ID | GACCAUAGAAGAGUUGAAAGA | UCUUUCAACUCUUCUAUGGUCUU |
| NO 1251 and 1252 | | |
| CC3_30; SEQ ID | CAACAUAGAGCUGUUGGAUGA | UCAUCCAACAGCUCUAUGUUGUU |
| NO 1253 and 1254 | | |
| CC3_31; SEQ ID | AUCGGAAGAGACCAAGCAAAA | UUUUGCUUGGUCUCUUCCGAUUU |
| NO 1255 and 1256 | | |
| CC3_32; SEQ ID | AUGGAAAGAAGGAUGGACAAA | UUUGUCCAUCCUUCUUUCCAUUU |
| NO 1257 and 1258 | | |
| CC3_33; SEQ ID | CUGCAUAAAGGCCUUCAUAGA | UCUAUGAAGGCCUUUAUGCAGUU |
| NO 1259 and 1260 | | |
| CC3_34; SEQ ID | CUCUACGAAGGUCAUGAACAU | AUGUUCAUGACCUUCGUAGAGUU |
| NO 1261 and 1262 | | |
| CC3_35; SEQ ID | GGCCUCUGGAGUAGAUAGAUA | UAUCUAUCUACUCCAGAGGCCUU |
| NO 1263 and 1264 | | |
| CC3_36; SEQ ID | UGGAGAAAGCAGUGAUGGUAA | UUACCAUCACUGCUUUCUCCAUU |
| NO 1265 and 1266 | | |
| CC3_37; SEQ ID | GUGAGGAUGGUAUGCGGGAUA | UAUCCCGCAUACCAUCCUCACUU |
| NO 1267 and 1268 | | |
| CC3_38; SEQ ID | UGCCAGAAGGAAUGAGAAUCA | UGAUUCUCAUUCCUUCUGGCAUU |
| NO 1269 and 1270 | | |
| CC3_39; SEQ ID | CUCCAAGUACGAGAUGAACAA | UUGUUCAUCUCGUACUUGGAGUU |
| NO 1271 and 1272 | | |
| CC3_40; SEQ ID | CACAGAAUCUAUGGUGGUUUA | UAAACCACCAUAGAUUCUGUGUU |
| NO 1273 and 1274 | | |
| S01_OR | mC*mA*mGmAmUmCfCfA | mU*fC*mUfUmGfGmUfGmAfA |
| | fCmUmUmCmAmCmCmAm | mGfUmGfGmAfUmC*fU*mG |
| | A*mG*mA | |

Notes:
fA, fU, fC, fG = 2′-deoxy-2′-fluro sugar base
mA, mU, mC, mG = 2′ Methyl sugar base
A, T, C, G = DNA bases
rU, rG, rC, rA = RNA bases
*= phosphorothioate (PS) linkages
In order to increase stability of the inhibitory RNA the first three nucleotides and the last three nucleotides can be connected via a PS linkage

### REFERENCES

Nair, J. K., Willoughby, J. L., Chan, A., Charisse, K., Alam, M. R., Wang, Q., Hoekstra, M., Kandasamy, P., Kel′in, A. V., Milstein, S. and Taneja, N., 2014. Multivalent N-acetylgalactosamine-conjugated siRNA localizes in hepatocytes and elicits robust RNAi-mediated gene silencing. Journal of the American Chemical Society, 136 (49), pp. 16958-16961.
Soutschek, J., Akinc, A., Bramlage, B., Charisse, K., Constien, R., Donoghue, M., Elbashir, S., Geick, A., Hadwiger, P., Harborth, J. and John, M., 2004. Therapeutic silencing of an endogenous gene by systemic administration of modified siRNAs. Nature, 432 (7014), p.173.

1. A nucleic acid molecule comprising:
a first part that comprises a double stranded inhibitory ribonucleic acid (RNA) molecule comprising a sense strand and an antisense strand; and
a second part that comprises a single stranded deoxyribonucleic acid (DNA) molecule, wherein the 3′ end of said single stranded DNA molecule is covalently linked to the 5′ end of the sense strand of the double stranded inhibitory RNA molecule or wherein the 3′ end of the single stranded DNA molecule is covalently linked to the 5′ of the antisense strand of the double stranded inhibitory RNA molecule characterized in that the double stranded inhibitory RNA comprises a sense nucleotide sequence that encodes a part of a human complement component gene and wherein said single stranded DNA molecule comprises a nucleotide sequence that is adapted over at least part of its length to anneal by complementary base pairing to a part of said single stranded DNA to form a double stranded DNA structure comprising a stem and a loop domain and said nucleic acid molecule is linked to N-acetylgalactosamine and said double stranded inhibitory RNA molecule consists of natural nucleotides.

2. The nucleic acid molecule according to claim 1 wherein the 3′ end of said single stranded DNA molecule is covalently linked to the 5′ end of the sense strand of the double stranded inhibitory RNA molecule.

3. The nucleic acid molecule according to claim 1 wherein the 3′ end of said single stranded DNA molecule is covalently linked to the 5′ end of the antisense strand of the double stranded inhibitory RNA molecule.

4. The nucleic acid molecule according to claim 1 wherein said loop domain comprises the nucleotide sequence GCGAAGC.

5. The nucleic acid molecule according to claim 4 wherein said single stranded DNA molecule comprises the nucleotide sequence
(SEQ ID NO 1)
5′TCACCTCATCCCGCGAAGC 3′
or
(SEQ ID NO 150)
5′ CGAAGCGCCCTACTCCACT 3′
or
(SEQ ID NO: 1155)
5′ GCGAAGCCCCTACTCCACT 3′.

6. (canceled)

7. The nucleic acid molecule according to claim 1 wherein said sense and/or said antisense strands comprises internucleotide phosphorothioate linkages.

8. The nucleic acid molecule according to claim 1 wherein said single stranded DNA molecule comprises one or more internucleotide phosphorothioate linkages.

9. (canceled)

10. The nucleic acid molecule according to claim 1 wherein said vinylphosphonate modification is to the 5′ terminal phosphate of said antisense RNA strand.

11. The nucleic acid molecule according to claim 1 wherein said double stranded inhibitory RNA molecule comprises 19 to 23 contiguous nucleotides in length.

12. The nucleic acid molecule according to claim 1 wherein N-acetylgalactosamine is monovalent, divalent, or trivalent.

13. The nucleic acid molecule according to claim 1 wherein said double stranded inhibitory RNA molecule comprises or consists of between 19 and 23 contiguous nucleotides of the sense nucleotide sequence set forth in SEQ ID NO: 67 (C5).

14. The nucleic acid molecule according to claim 13 wherein said C5 double stranded inhibitory RNA molecule comprises a nucleotide sequence selected from the group consisting of: SEQ ID NO: 119, 120, 121, 122, 123, 124, 125, 126, 127, 128, 129, 130, 131, 132, 133, 134, 135, 136, 137 and 138.

15. The nucleic acid molecule according to claim 13 wherein said C5 double stranded inhibitory RNA molecule comprises a nucleotide sequence selected from the group consisting of: SEQ ID NO: 109, 110, 111, 112, 113, 114, 115, 116, 117 and 118.

16. The nucleic acid molecule according to claim 13 wherein said C5 double stranded inhibitory RNA molecule comprises a nucleotide sequence selected from the group consisting of: SEQ ID NO: 139, 140, 141, 142, 143, 144, 145, 146, 147 and 148.

17. The nucleic acid molecule according to claim 13 wherein said C5 double stranded inhibitory RNA molecule comprises an antisense nucleotide sequence selected from the group consisting of: SEQ ID NO: 31, 32, 33, 34, 35, 36, 37, 38, 39 and 40.

18. The nucleic acid molecule according to claim 13 wherein said C5 double stranded inhibitory RNA molecule comprises a sense nucleotide sequence selected from the group consisting of: SEQ ID NO: 21, 22, 23, 24, 25, 26, 27, 28, 29 and 30.

19. The nucleic acid molecule according to claim 1 wherein said double stranded inhibitory RNA molecule comprises or consists of between 19 and 23 contiguous nucleotides of the sense nucleotide sequence set forth in SEQ ID NO: 66 (C3).

20. The nucleic acid molecule according to claim 19 wherein said C3 double stranded inhibitory RNA molecule comprises a nucleotide sequence selected from the group consisting of: SEQ ID NO: 79, 80, 81, 82, 83, 84, 85, 86, 87, 88, 89, 90, 91, 92, 93, 94, 95, 96, 97, 98 and 99.

21. The nucleic acid molecule according to claim 19 wherein said C3 double stranded inhibitory RNA molecule comprises a nucleotide sequence selected from the group consisting of: SEQ ID NO: 69, 70, 71, 72, 73, 74, 75, 76, 77 and 78.

22. The nucleic acid molecule according to claim 19 wherein said C3 double stranded inhibitory RNA molecule comprises a nucleotide sequence selected from the group consisting of: SEQ ID NO: 100, 101, 102, 103, 104, 105, 106, 107 and 108.

23. The nucleic acid molecule according to claim 19 wherein said C3 double stranded inhibitory RNA molecule comprises an antisense nucleotide sequence selected from the group consisting of: SEQ ID NO: 11, 12, 13, 14, 15, 16, 17, 18, 19 and 20.

24. The nucleic acid molecule according to claim 19 wherein said C3 double stranded inhibitory RNA molecule comprises a sense nucleotide sequence selected from the group consisting of: SEQ ID NO: 61, 2, 3, 4, 5, 6, 7, 8, 9 and 10.

25. The nucleic acid molecule according to claim 19 wherein said C3 double stranded inhibitory RNA molecule comprises a sense nucleotide sequence selected from the group consisting of: SEQ ID NO: 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271 and 1273.

26. The nucleic acid molecule according to claim 19 wherein said C3 double stranded inhibitory RNA molecule comprises an antisense nucleotide sequence selected from the group consisting of: SEQ ID NO: 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272 and 1274.

27. The nucleic acid molecule according to claim 1 wherein said double stranded inhibitory RNA molecule comprises or consists of between 19 and 23 contiguous nucleotides of the sense nucleotide sequence set forth in SEQ ID NO: 68 (MASP2).

28. The nucleic acid molecule according to claim 27 wherein said MASP2 double stranded inhibitory RNA molecule comprises an antisense nucleotide sequence selected from the group consisting of: SEQ ID NO: 51, 52, 53, 54, 55, 56, 57, 58, 59 or 60.

29. The nucleic acid molecule according to claim 27 wherein said MASP2 double stranded inhibitory RNA molecule comprises a sense nucleotide sequence selected from the group consisting of: SEQ ID NO: 41, 42, 43, 44, 45, 46, 47, 48, 49 and 50.

30. The nucleic acid molecule according to claim 1 wherein the 3′ end of said single stranded DNA molecule is covalently linked to the 5′ end of the sense strand of the double stranded inhibitory RNA molecule and said N-acetylgalactosamine is linked to the 3′ end of said sense strand of the double stranded inhibitory RNA molecule.

31. A pharmaceutical composition comprising at least one nucleic acid molecule according to claim 1 further comprising a pharmaceutical carrier and/or excipient.

32. A method of treating a disease or condition that would benefit from inhibition of complement activation in a patient comprising administering an effective amount of a nucleic acid molecule according to claim 1.
